(19) United States
(12) Patent Application Publication
(10) Pub. No.: US 2008/0269293 A1
(43) Pub. Date: Oct. 30, 2008

(54) PYRAZOLE DERIVATIVES AS ANTI-PLATELET AND ANTI-THROMBOTIC AGENTS

(75) Inventors: Liguo Chi, Ann Arbor, MI (US); Chulho Choi, Mystic, CT (US); Andrew G. Geyer, Chicago, IL (US); Robert M. Kennedy, Ann Arbor, MI (US); Jeffery A. Pfefferkorn, Mystic, CT (US); Roy T. Winters, Pinckney, MI (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 11/843,431

(22) Filed: Aug. 22, 2007

Related U.S. Application Data
(60) Provisional application No. 60/940,871, filed on May 30, 2007.
(60) Provisional application No. 60/823,519, filed on Aug. 25, 2006.

(51) Int. Cl. A61K 31/4152; C07D 231/22; A61K 31/4439; A61P 9/10; A61P 7/02; A61K 31/4453; C07D 401/12

(52) U.S. Cl. 514/326; 548/371.1; 546/276.1; 546/211; 514/407; 514/341

(57) ABSTRACT

This invention relates to novel compounds of formula (I)

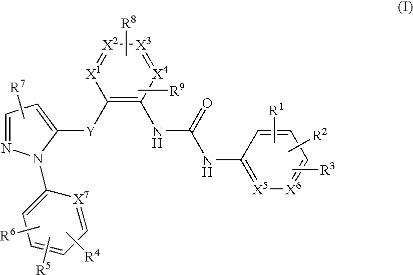

or stereoisomers or pharmaceutically acceptable salts thereof wherein Y, R¹ through R⁹, and X¹ through X⁷ are as defined in the specification, pharmaceutical compositions containing said compounds useful as P2Y₁ antagonists, and to methods of treating thromboembolic disorders.

### BACKGROUND OF THE INVENTION

The present invention relates to N-phenyl and N-pyridyl pyrazole derivatives. The invention also relates to the pharmaceutically acceptable salts of such compounds, processes for the preparation of the compounds, pharmaceutical compositions containing the compounds and uses of the compounds in treating thromboembolic disorders.

The compounds of the present invention are antagonists of P2Y₁ and have a number of therapeutic applications, particularly in the modulation of platelet reactivity, in the treatment of thromboembolic disorders, and other disease states which are responsive to modulation of P2Y₁ activity.

Purinoreceptors bind to and are activated by a variety of both ribosylated (nucleotide) and non-ribosylated (nucleoside) purines. This distinction has been used to classify these receptors into two broad groups: the P1 receptors (A1, A2a, A2b and A3), which bind to and are activated by the nucleoside adenosine, and the P2 receptors, which comprise a second, more diverse class of receptors which are activated by a wide variety of nucleotides including ATP, ADP, UTP and UDP. The P2 receptors can be further subdivided into two distinct types of receptors; the ionotropic P2X receptors that mediate cation flux across cellular membranes in response to ATP and the metabotropic P2Y family of receptors which are G-protein coupled receptors. In humans, the P2Y family of receptors is generally considered to consist of seven distantly related members; P2Y₁, P2Y₂, P2Y₄, P2Y₆, P2Y₁, P2Y₁₂, and P2Y₁₃ (Boeynaems, J. M. et al. Drug Development Research 2000, 52, 187-9). In addition, an eighth receptor, P2Y₁₄, has been considered by some to be a member of this class although it does not respond to ribosylated nucleotides and is activated by UDP-glucose (Abbracchio, M. P. et al. Trends Pharmacol. Sci. 2003, 24, 52-5).

Several studies have suggested that modulators of specific members of the P2Y family of receptors could have therapeutic potential for the treatment of a variety of disorders (for review, see Burnstock, G. and Williams, M. J. Pharm. Exp Ther. 2000, 295, 862-9), including diabetes, cancer, CF, and treatment of ischemia-reperfusion injury (Abbracchio M. P., Burnstock G. Pharmacol. Ther. 1994, 64, 445-475). P2Y1 receptors, almost ubiquitous among human organs (Jassens R; Communi D.; Pirotton S. et al. Biochem. Biophys. Res. Comm. 1996, 221, 588-593), have been identified on microglia (Norenberg W. et al.; Br. J. Pharmacol. 1994, 111, 942-950) and on astrocytes (Salter M. W. and Hicks J. L. J. Neurosc. 1995, 15, 2961-2971). Extracellular ATP activates microglial and/or astrocytes via P2Y receptors and leads directly to the release of inflammatory mediators. Microglia and astrocytes are believed to play a role in the progression of Alzheimer's disease and other CNS inflammatory disorders such as stroke and multiple sclerosis.

Two members of the P2Y family, P2Y₁ and P2Y₁₂, are of particular interest as they have now both been shown to act as important receptors for ADP in platelets (Jin, J. et al. Proc. Natl. Acad. Sci. 1998, 95, 8070). ADP is a key activator of platelets and platelet activation is known to play a pivotal role in thrombus formation under conditions of high shear stress such as those found in the arterial circulation. In addition, more recent data has suggested that platelet activation may also play a role in mediating thrombus formation under lower shear stress such as that found in the venous circulation. ADP activates platelets by simultaneously interacting with both P2Y₁ and P2Y₁₂ to produce two separate intracellular signals which synergize together to produce complete platelet activation. The first signal arises from ADP driven activation of the P2Y₁ receptor and can most easily be tracked by measuring the transitory increase in intracellular free Ca⁺². This signal appears to mediate the initial shape change reaction and to initiate the process of platelet activation. The second signal appears to be derived from ADP activation of the P2Y₁₂ receptor and serves to consolidate the process and produce an irreversible platelet aggregate. Using three structurally related but distinct inhibitors of P2Y₁ (A3P5P, A3P5PS and A2P5P), Daniel, J. L. et al. (J. Biol. Chem. 1998, 273, 2024-9), Savi, P. et al. (FEBS Letters 1998, 422, 291-5), and Hechler, B. et al. (Br. J. Haematol. 1998, 103, 858-66) were the first to publish the observation that the inhibition of P2Y₁ activity alone could block ADP-driven aggregation independently of the P2Y₁₂ receptor. Although inhibition of platelet reactivity is often thought of as firm evidence of an anti-thrombotic activity, these antagonists lacked the necessary pharmacological properties for in vivo study. The first direct demonstration that inhibition of P2Y₁ activity could lead to an anti-thrombotic effect in vivo was reported by Leon, C. et al. Circulation 2001, 103, 718-23, in a model of thromboplastin induced thromboembolism using both a P2Y₁ knock-out mouse and the P2Y₁ antagonist MRS-2179 (Baurand, A. and Gachet, C. Cardiovascular Drug Reviews 2003, 21, 67-76). These results were subsequently extended to include the inhibition of both venous and arterial thrombosis in the rat (Lenain, N. et al. J. Thromb. Haemost. 2003, 1, 1144-9) and confirmed by a second laboratory using an independently derived P2Y₁ knock-out mouse (Fabre, J-E. et al. Nature Medicine 1999, 5, 1199-1202). Taken together, these data suggest that the discovery of novel P2Y₁ antagonists with improved pharmaceutical characteristics could have significant utility in the treatment of a variety of thromboembolic disorders.

### SUMMARY OF THE INVENTION

The present invention relates to a compound of formula (I)

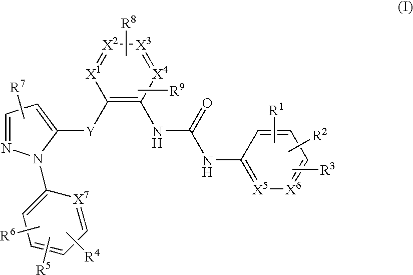

wherein

X¹, X², X³, X⁴, X⁵, X⁶, and X⁷ are each independently CH or N, with the proviso that no more than two of X¹, X², X³, X⁴ can be N at the same time;

Y is oxy or thio;

R¹, R², R³, R⁴, R⁵ and R⁶ are each independently —H, C₁-C₆ alkyl, C₅-C₈ cycloalkyl, cycloheteroalkyl, hydroxy, C₁-C₆ alkoxy, halo, —CF₃, —CF₂CF₃, —OCF₃, —OCF₂CF₃, —OCF₂CF₂H, optionally substituted phenyl, —SiMe₃, —(CR¹⁰R¹¹), —OR¹², —SR¹³, —CN, —NO₂, —(CR¹⁰R¹¹)ₙNR¹⁴R¹⁵, —(CR¹⁰R¹¹), —C(O)R¹², —(CR¹⁰R¹¹)ₙ—CO₂R¹², —(CR¹⁰R¹¹)ₙ—C(O)—NR¹⁴R¹⁵, or —S(O)ₚR¹⁶;

R⁷ is —H, C₁-C₄ alkyl, halo, —CF₃, or —(CR¹⁰R¹¹)ₙ—CO₂R¹².

R⁸ and R⁹ are each independently —H, C₁-C₆ alkyl, hydroxy, C₁-C₆ alkoxy, halo, —CF₃, or —SR¹³ and are each only bound to a carbon atom;

R¹⁰ and R¹¹ are each independently at each occurrence —H, C₁-C₄ alkyl, or halo;

R¹² and R¹³ are each independently at each occurrence —H or C₁-C₆ alkyl;

R¹⁴ and R¹⁵ are each independently at each occurrence —H, C₁-C₆ alkyl, —C(O)(C₁-C₆ alkyl), —S(O)ₚ(C₁-C₆ alkyl), or R¹⁴ and R¹⁵ taken together in combination with the nitrogen to which they are attached combine to form a piperidinyl or pyrrolidinyl ring;

R¹⁶ is —H, C₁-C₄ alkyl;

n, at each occurrence, is selected from 0, 1, 2, 3, and 4; and

p, at each occurrence, is selected from 0, 1, and 2;

or a stereoisomer or a pharmaceutically acceptable salt thereof.

The present invention also relates to a pharmaceutical composition comprising a compound of formula (I), or a pharmaceutically acceptable salt thereof, and a pharmaceutically acceptable carrier.

Another embodiment of the invention relates to a method for modulation of platelet reactivity in a patient in need thereof comprising administering to said patient a therapeutically effective amount of a compound of formula (I), or a pharmaceutically acceptable salt thereof.

A further embodiment of the invention provides a method of treating a thromboembolic disorder in a patient in need thereof comprising administering to said patient a therapeutically effective amount of a compound of formula (I) or a pharmaceutically acceptable salt thereof.

The present invention further provides a process for making the compounds of the present invention or a pharmaceutically acceptable salt thereof.

### DETAILED DESCRIPTION OF THE INVENTION

As used in this application:

a) the term “halogen” or “halo” refers to a fluorine atom, chlorine atom, bromine atom, or iodine atom;

b) the term “C₁-C₆ alkyl” refers to a branched or straight chained alkyl radical containing from 1 to 6 carbon atoms, such as methyl, ethyl, n-propyl, isopropyl, n-butyl, isobutyl, sec butyl, t-butyl, pentyl, hexyl, and the like;

c) the term “C₁-C₄ alkyl” refers to a branched or straight chained alkyl radical containing from 1 to 4 carbon atoms, such as methyl, ethyl, n-propyl, isopropyl, n-butyl, isobutyl, sec-butyl, t-butyl, and the like;

d) the term “C₅-C₈ cycloalkyl” refers to a cyclic alkyl radical containing from 5 to 8 carbon atoms such as cyclopentyl, cyclohexyl, cycloheptyl, and cyclooctyl;

e) the term “cycloheteroalkyl” refers to C₅-C₈ cycloalkyl where one of the carbon atoms in the ring has been substituted with an oxygen, sulfur, or nitrogen atom, such as pyrrolidine, piperidine, tetrahydrofuran, tetrahydrothiophene, tetrahydropyran, and the like;

f) the term “C₁-C₆ alkoxy” refers to a straight or branched alkoxy group containing from 1 to 6 carbon atoms, such as methoxy, ethoxy, n-propoxy, isopropoxy, n-butoxy, isobutoxy, sec-butoxy, t-butoxy, pentoxy, hexoxy, and the like;

g) the term “C₁-C₄ alkoxy” refers to a straight or branched alkoxy group containing from 1 to 4 carbon atoms, such as methoxy, ethoxy, n-propoxy, isopropoxy, n-butoxy, isobutoxy, sec-butoxy, t-butoxy, etc;

h) the term “optionally substituted” as used herein means an optional substitution of one to three, preferably one or two groups independently selected from halo, hydroxy, cyano, nitro, C₁-C₄ alkyl, and C₁-C₄ alkoxy;

i) the designation “ [symbol] ” or refers to a bond for which the stereochemistry is not designated;

j) the designation “ [symbol] ” refers to a bond that protrudes forward out of the plane of the page;

k) the designation “ [symbol] ” refers to a bond that protrudes backward out of the plane of the page;

l) as used in the preparations and examples the following terms have the indicated meanings; “ng” refers to nanograms; “μg” refers to micrograms; “mg” refers to milligrams; “g” refers to grams; “kg” refers to kilograms; “nmole” or “inmol” refers to nanomoles; “mmol” refers to millimoles; “mol” refers to moles; “μL” refers to microliters; “mL” refers to milliliters; “L” refers to liters; “° C.” refers to degrees Celsius; “bp” refers to boiling point; “mm of Hg” refers to pressure in millimeters of mercury; “mp” refers to melting point; “nM” refers to nanomolar; “μM” refers to micromolar; “mM” refers to millimolar; “M” refers to molar; “psi” refers to pounds per square inch; “rpm” refers to revolutions per minute; “hrs” refers to hours; “HPLC” refers to high performance liquid chromatography; “RP-HPLC” refers to reverse phase high performance liquid chromatography; “HRMS” refers to high resolution mass spectrum; “; “DMSO” refers to dimethyl sulfoxide, “brine” refers to a saturated aqueous solution of sodium chloride; “μCi” refers to microcuries; “i.p.” refers to intraperitoneally; “i.v.” refers to intravenously; “DMF” refers to N,N-dimethylformamide; “EtOH” refers to ethanol; “LC/MS” refers to liquid chromatography/mass spectometry; “MeOH” refers to methanol; “THF” refers to tetrahydrofuran; “eq” refers to equivalent; “M” refers to molar (mol/l); “N” refers to normality (Eq/l); “soln” refers to solution; “temp” refers to temperature; “conc” refers to concentrate; “vac” refers to vacuum;

m) the designation “—C(O)—” or “C(O)” refers to a carbonyl group of the formula:

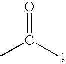

n) “—NR¹⁴R¹⁵” refers to an amine of the formula:

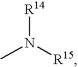

wherein R¹⁴ and R¹⁵ are each independently —H, C₁-C₆ alkyl, —C(O)(C₁-C₆ alkyl), —S(O)ₚ(C₁-C₆ alkyl), or R¹⁴ and R¹⁵ taken together in combination with the nitrogen to which they are attached combine to form a piperidinyl or pyrrolidinyl ring;

o) the term “enantiomeric excess” or “ee” refers to the percent by which one enantiomer, E1 is in excess in a mixture of the two enantiomers, E1 plus E2, such that

{(E1−E2)/(E1+E2)}×100%=ee.

Pharmaceutically acceptable salts of the compounds of formula I include the acid addition and base salts (including disalts) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

The compounds of the present invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985).

Some examples of such prodrugs include:

(i) where the compound of formula (I) contains a carboxylic acid functionality (—COOH), an ester thereof, for example, replacement of the hydrogen with (C₁-C₈)alkyl;

(ii) where the compound of formula (I) contains an alcohol functionality (—OH), an ether thereof, for example, replacement of the hydrogen with (C₁-C₆)alkanoyloxymethyl; and

(iii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

As with any group of structurally related compounds which possesses a particular utility, certain groups and configurations are preferred for the compounds of formula (I) and their end-use application.

Preferred embodiments of compounds of formula (I) or stereoisomers or pharmaceutically acceptable salts thereof are given below:

(1) Compounds in which Y is oxy;

(2) Compounds in which:
(a) R¹ is C₁-C₆ alkyl, cycloheteroalkyl, optionally substituted phenyl, —F, —Cl, —Br, —I, —OCF₃, —OCF₂CF₃, —OCF₂CF₂H, —SR¹³, or —(CR¹⁰R¹¹)ₙ—CO₂R¹²;
(b) R² and R³ are —H, —F, —Cl, methyl, or methoxy;
(c) R¹ is C₁-C₆ alkyl, —OCF₃, —SR¹³, or —(CR¹⁰R¹¹)ₙ—CO₂R¹²; R² is —H, —F, —C₁-C₆ alkyl, C₁-C₆ alkoxy, or —CF; and R³ is —H;
(d) Compounds in which R¹ is C₁-C₆ alkyl, —OCF₃, —SR¹³, —COOH, or —COOCH₃; R² is —H, —F, C₁-C₆ alkyl, C₁-C₆ alkoxy, or —CF; and R³ is —H;
(e) Compounds where R¹ is —OCF₃ or t-butyl; R² is —H, —F, methyl, methoxy, or ethoxy; and R³ is —H;
(f) Compounds where R¹ is —OCF₃ or t-butyl and both R² and R³ are —H;

(3) Compounds in which:
(a) R⁴ is —H, C₁-C₆ alkyl, C₅-C₈ cycloalkyl, C₁-C₆ alkoxyl, —F, —Cl, —Br, —I, —CF₃, —CF₂CF₃, optionally substituted phenyl, or —(CR¹⁰R¹¹)ₙ—CO₂R¹²;
(b) Both R⁵ and R⁶ are —H;
(c) R⁴ is —H, C₁-C₆ alkyl, C₅-C₈ cycloalkyl, C₁-C₆ alkoxyl, —F, —Cl, —Br, —I, —CF₃, —CF₂CF₃, or —(CR¹⁰R¹¹)—CO₂R¹²; R⁵ is —H, —F, or —Cl; and R⁶ is —H;
(d) R⁴ is C₁-C₆ alkyl, C₁-C₆ alkoxyl —F, —Cl, —Br, —I, —CF₃, —CF₂CF₃, or —(CR¹⁰R¹¹)ₙ—CO₂R¹² and both R⁵ and R⁶ are —H;
(e) R⁴ is methyl, ethyl, methoxy, ethoxy, —F, —Cl, or —CF₃ and both R⁵ and R⁶ are —H;

(4) Compounds in which:
(a) R⁷ is —H or C₁-C₄ alkyl;
(b) R⁷ is —(CR¹⁰R¹¹)ₙ—CO₂R¹²;
(c) R⁷ is —H, methyl, —COOH, or —COOCH₃;

(5) Compounds in which R⁸ and R⁹ are both —H;

(6) Compounds in which:
(a) X¹, X², X³, X⁴, X⁵, X⁶, and X⁷ are all CH;
(b) X¹ is N;
(c) X³ is N;
(d) X¹ is N; and X², X³, X⁴, X⁵, X⁶ and X⁷ are all CH;
(e) X³ is N; and X¹, X², X⁴, X⁵, X⁶ and X⁷ are all CH;

(7) Compounds in which R¹⁰ and R¹¹ are both —H in all occurrences;

(8) Compounds in which R¹² and R¹³ are both —H in all occurrences.

It is understood that further preferred embodiments of formula (I) can be selected by requiring one or more of the preferred embodiments (1) through (6) above of compounds of formula (I) or stereoisomers, pharmaceutically acceptable salts, or prodrugs thereof or by reference to the examples given herein. For example, further preferred embodiments of the invention can be obtained by combining (1) and (2)(a); (1), (2)(a), and (3)(c); (1), (2)(c), and (3)(c); (2)(d) and (3)(d); (2)(b), (3)(e), (4)(a), and (5); (1), (2)[(a) through (f)], (3)(e), (4)(a), (5), and (6)(a); (1), (2)[(a) through (f)], (3)(e), (4)(a), (5), and (6)(d); (1), (2)[(a) through (f)], (3)(e), (4)(a), (5), and (6)(e); (2)[(a) through (f)], (3)(e), (4)(a), (5), and (6)(a); (1), (2)(d), (3)(e), (4)(a), (5), and (6)(a); (2)(d), (3)(e), (4)(a), (5), and (6)[(a) through (e)]; (2)(d), (3)(e), (4)(a), (5), (6)[(a) through (e)], and (7); (2)(d), (3)(e), (4)(a), (5), (6)[(a) through (e)], and (8); (2)(d), (3)(e), (4)(a), (5), (6)[(a) through (e)], (7), and (8); (2)(a), and (6)(a); (5) and 6(a), or by solely requiring (1); (2)(a); (2)(d); (4)(a); (5); (6)(a); (7), (8), and the like. It is further understood that the stereoisomers and pharmaceutically acceptable salts are included in the term “compound” unless specifically disclaimed.

Additional embodiments of the invention are represented by compounds of formula (IA) or stereoisomers or pharmaceutically acceptable salts thereof:

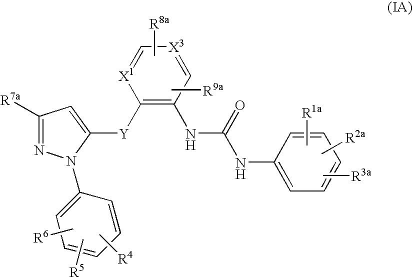

wherein X and X³ are each independently CH or N; Y is oxy or thio; R^{1a}, R^{2a}, and R^{3a} are each independently —H, C₁-C₆ alkyl, C₁-C₆ alkoxy, halo, —CF₃, —OCF₃, —SR¹³, and —(CR¹⁰R¹¹)ₙ—CO₂R¹²; R⁴, R⁵, and R⁶ are each independently —H, C₁-C₆ alkyl, C₅-C₈ cycloalkyl, cycloheteroalkyl, hydroxy, C₁-C₆ alkoxy, halo, —CF₃, —CF₂CF₃, —OCF₃, —OCF₂CF₃, —OCF₂CF₂H, optionally substituted phenyl, —SiMe₃, —(CR¹⁰R¹¹)ₙ—OR¹²—SR¹³, —CN, —NO₂, —(CR¹⁰R¹¹)ₙNR¹⁴R¹⁵, —(CR¹⁰R¹¹)ₙ—C(O)R¹²—(CR¹⁵R¹⁰)ₙ—CO₂R¹²—(CR¹⁰R¹¹)ₙ—C(O)—NR¹⁴R¹⁵ or —S(O)ₚR¹⁶; R^{7a} is —H, C₁-C₄ alkyl, halo, or —CF₃; R^{8a} and R^{9a} are each independently —H, C₁-C₆ alkyl, hydroxy, C₁-C₆ alkoxy, halo, or —CF₃; R¹⁰ and R¹¹ are each independently at each occurrence —H, C₁-C₄ alkyl, or halo; R¹² and R¹³ are each independently at each occurrence —H or C₁-C₆ alkyl; R¹⁴ and R¹⁵ are each independently at each occurrence —H, C₁-C₆ alkyl, —C(O)(C₁-C₆ alkyl), —S(O)ₚ(C₁-C₆ alkyl), or R¹⁴ and R¹⁵ taken together in combination with the nitrogen to which they are attached combine to form a piperidinyl or pyrrolidinyl ring; R¹⁶ is —H, C₁-C₄ alkyl; n, at each occurrence, is selected from 0, 1, 2, 3, and 4; and p, at each occurrence, is selected from 0, 1, and 2.

Preferred embodiments of compounds of formula (IA) or stereoisomers or pharmaceutically acceptable salts thereof are given below:

(1) Compounds in which Y is oxy;

(2) Compounds in which:
(a) R^{1a} is C₁-C₆ alkyl, —F, —Cl, —Br, —I, —OCF₃, —SR¹³ or —(CR¹⁰R¹¹)ₙ—CO₂R¹²;
(b) R^{2a} and R^{3a} are each independently —H, —F, —Cl, methyl, or methoxy; or
(c) R^{1a} is —C₁-C₆ alkyl, —F, —Cl, —OCF₃, —SR¹³, or —(CR¹⁰R¹¹)ₙ—CO₂R¹²; and both R^{2a} and R^{3a} are —H;

(3) Compounds in which:
(a) R⁴ is —H, C₁-C₆ alkyl, C₅-C₈ cycloalkyl, C₁-C₆ alkoxyl, —F, —Cl, —Br, —I, —CF₃, —CF₂CF₃, optionally substituted phenyl, or —(CR¹⁰R¹¹)ₙ—CO₂R¹²;
(b) Both R⁵ and R⁶ are —H;
(c) R⁴ is C₁-C₆ alkyl, C₅-C₈ cycloalkyl, C₁-C₆ alkoxyl, —F, —Cl, —Br, —I, —CF₃, —CF₂CF₃, optionally substituted phenyl or —(CR¹⁰R¹¹), —CO₂R¹²; R⁵ is —H, —F, or —Cl; and R⁶ is —H;
(d) R⁴ is C₁-C₆ alkyl, C₅-C₈ cycloalkyl, C₁-C₆ alkoxyl —F, —Cl, —Br, —I, —CF₃, —CF₂CF₃, phenyl or —(CR¹⁰R¹¹)ₙ—CO₂R¹² and both R⁵ and R⁶ are —H;
(e) R⁴ is methyl, ethyl, methoxy, ethoxy, —F, —Cl, or —CF₃ and both R⁵ and R⁶ are —H;

(4) Compounds in which Compounds in which R^{8a} a and R^{9a} are both —H;

(5) Compounds in which:
(a) X¹ and X³ are both CH;
(b) X¹ is N;
(c) X³ is N;
(d) X¹ is N and X³ is CH;
(e) X³ is N and X¹ is CH;

(6) Compounds in which R¹⁰ and R¹¹ are both —H in all occurrences;

(7) Compounds in which R¹² and R¹³ are both —H in all occurrences.

It is understood that further preferred embodiments of formula (IA) can be selected by requiring one or more of the preferred embodiments (1) through (7) above of compounds of formula (IA) or stereoisomers, pharmaceutically acceptable salts, or prodrugs thereof or by reference to the examples given herein. For example, further preferred embodiments of the invention can be obtained by combining (1) and (2)(a); (1), (2)(a), and (3)(c); (2)(a), and (3)(b); (2)(c) and (3)(e); (2)(b), (3)(d), and (4); (2)(c), (3)(e), (4), and (5)(a); (2)(c), (3)(e), (4), and (5)(b); (2)(c), (3)(e), (4), and (5)(c); (2)(c), (3)(e), (4), (5)(a), and (6); (2)(c), (3)(e), (4), and (5)(c); (2)(c), (3)(e), (4), (5)(a), and (7); (2)(c), (3)(e), (4), and (5)(c); (2)(c), (3)(e), (4), (5)(a), (6), and (7); or by solely requiring (2)(c); (3)(a); (3)(e); (5)(a); (5)(c); (6); (7); and the like. It is further understood that the stereoisomers and pharmaceutically acceptable salts are included in the term “compound” unless specifically disclaimed.

Further embodiments of the invention are represented by compounds of formula (IB) or stereoisomers or pharmaceutically acceptable salts thereof:

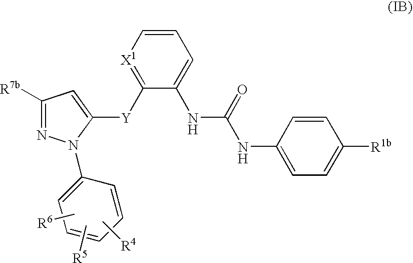

wherein X¹ is CH or N; Y is oxy or thio; R^{1b} is —H, C₁-C₆ alkyl, C₁-C₆ alkoxy, halo, —CF₃, —OCF₃, —SR¹³, and —(CR¹⁰R¹¹)—CO₂R¹², R⁴, R⁵, and R⁶ are each independently —H, C₁-C₆ alkyl, C₅-C₈ cycloalkyl, cycloheteroalkyl, hydroxy, C₁-C₆ alkoxy, halo, —CF₃, —CF₂CF₃, —OCF₃, —OCF₂CF₃, —OCF₂CF₂H, optionally substituted phenyl, —SiMe₃, —(CR¹⁰R¹¹), —OR¹², —SR¹³, —CN, —NO₂, —(CR¹⁰R¹¹)ₙNR¹⁴R¹⁵, —(CR¹⁰R¹¹)ᵣ—C(O)R¹², —(CR¹⁰R¹¹)ₙ—CO₂R¹², —(CR¹⁰R¹¹)ₙ—C(O)—NR¹⁴R¹⁵, or —S(O)ₚR¹⁶; R^{7b} is —H, C₁-C₄ alkyl, halo, or —CF₃; R¹⁰ and R¹¹ are each independently at each occurrence —H, C₁-C₄ alkyl, or halo; R¹² and R¹³ are each independently at each occurrence —H or C₁-C₆ alkyl; R¹⁴ and R¹⁵ are each independently at each occurrence —H, C₁-C₆ alkyl, —C(O)(C₁-C₆ alkyl), —S(O)ₚ(C₁-C₆ alkyl), or R¹⁴ and R¹⁵, taken together in combination with the nitrogen to which they are attached combine to form a piperidinyl or pyrrolidinyl ring; R¹⁶ is —H, C₁-C₄ alkyl; n, at each occurrence, is selected from 0, 1, 2, 3, and 4; and p, at each occurrence, is selected from 0, 1, and 2.

Preferred embodiments of compounds of formula (IB) or stereoisomers or pharmaceutically acceptable salts thereof are given below:

(1) Compounds in which Y is oxy;

(2) Compounds in which:
(a) R^{1a} is C₁-C₆ alkyl, —F, —Cl, —Br, —I, —OCF₃, —SR¹³, or —(CR¹⁰R¹¹)ₙ—CO₂R¹²;
(b) R^{1a} is —OCF₃;
(c) R^{1a} is C₁-C₆ alkyl;

(3) Compounds in which:
(a) R⁴ is —H, —C₁-C₆ alkyl, C₅-C₈ cycloalkyl, I—C₁-C₆ alkoxyl, —F, —Cl, —Br, —I, —CF₃, —CF₂CF₃, optionally substituted phenyl, or —(CR¹⁰R¹¹)ₙ—CO₂R¹²;
(b) Both R⁵ and R⁶ are —H;
(c) R⁴ is —C₁-C₆ alkyl, —C₁-C₆ alkoxyl —F, —Cl, —Br, —I, —CF₃, —CF₂CF₃, or —(CR¹⁰R¹¹)—CO₂R¹²; R⁵ is —H, —F, or —Cl; and R⁶ is —H;
(d) R⁴ is —C₁-C₆ alkyl, —C₁-C₆ alkoxyl —F, —Cl, —Br, —I, —CF₃, —CF₂CF₃, or —(CR¹⁰R¹¹)ₙ—CO₂R¹² and both R⁵ and R⁶ are —H;
(e) R⁴ is methyl, ethyl, methoxy, ethoxy, —F, —Cl, or —CF₃ and both R⁵ and R⁶ are —H;

(4) Compounds in which:
(a) X¹ is CH;
(b) X¹ is N;

(5) Compounds in which R¹⁰ and R¹¹ are both —H in all occurrences;

(6) Compounds in which R¹² and R¹³ are both —H in all occurrences.

It is understood that further preferred embodiments of formula (IB) can be selected by requiring one or more of the preferred embodiments (1) through (6) above of compounds of formula (IB) or stereoisomers, pharmaceutically acceptable salts, or prodrugs thereof or by reference to the examples given herein. For example, further preferred embodiments of the invention can be obtained by combining (1) and (2)(a); (1), (2)(a), and (3)(c); (2)(b), and (3)(d); (2)(b) and (3)(e); (2)(b), (3)(d), and (4)(a); (2)(c), (3)(e), (4)(a), and (5); (2)(a), (3)(e), (4)(a), and (5); (2)(a), (3)(e), (4)(a), (5), and (6); (2)(a), (3)(a), (4)(b), and (5); (2)(a), (3)(a), (4)(b), (5), and (6); (2)(b), (3)(a), and (5)(a); or by solely requiring (2)(a); (2)(b); (2)(c); (3)(a); (3)(d); (3)(e); (4)(a); (4(b); (5); (6); and the like. It is further understood that the stereoisomers and pharmaceutically acceptable salts are included in the term “compound” unless specifically disclaimed.

For the purposes of this invention, compounds of formulae (IA) and (IB) are subsets of formula (I) and are included below in the definition of compounds of formula (I) when discussing how to make, formulate, and use the compounds of formula (I) unless otherwise indicated.

Compounds of formula (I) and intermediates thereof can be prepared in a number of ways known to one of ordinary skill in the art of organic synthesis and preferably as described in Schemes A and B. All the substituents, unless otherwise indicated, are as previously defined. The reagents and starting materials are readily available to one of ordinary skill in the art.

A particularly useful reference of synthetic methods which may be applicable to preparation of compounds of the present invention may be found in Larock, R. C. Comprehensive Organic Transformations, VCH: New York, 1989, hereby incorporated by reference. Another useful reference for choosing appropriate protecting groups used in the protection of the reactive functional groups present in the compounds described in this invention is Greene and Wuts, Protective Groups In Organic Synthesis, Wiley and Sons, 1991, also incorporated by reference herein as if fully set forth.

Scheme A provides a synthetic process for making compounds of formula (8) which represent compounds of formula (I) wherein Y is oxy and all of the remaining substituents are as defined in formula (I). The substituent R^{P1} is defined as C₁-C₆ alkyl, preferably methyl or ethyl. The substituent “Hal” is defined as halogen, preferably chloro or fluoro.

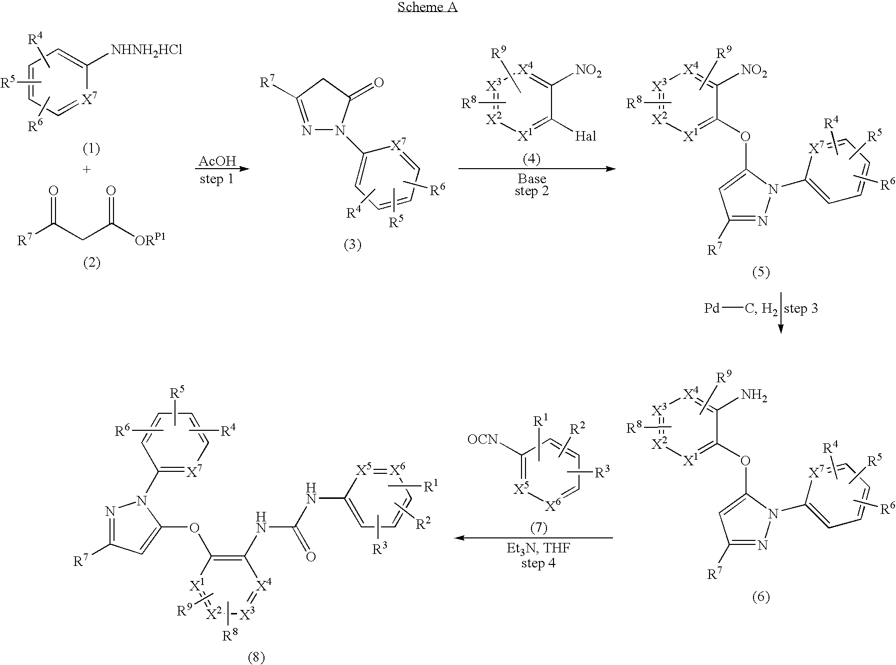

As illustrated in Scheme A, compounds of general structure (8), can be prepared by initial condensation of phenyl or pyridyl hydrazine (1) with β-ketoester (2) in acetic acid to afford the pyrazole of structure (3). Reaction of pyrazole (3) with the 2-halo-1-nitrobenzene or suitable heterocycle bearing a halogen and a nitro group in a 1,2 relationship as depicted by structure (4) in the presence of a base provides intermediate (5). Intermediate (5) can subsequently be converted to amine (6) by catalytic hydrogenation. Finally, reaction of amine (6) with the phenyl isocyanate of (7) will provide the N-phenyl pyrazole derivative of structure (8).

For example, in Scheme A, step 1, phenyl or pyridyl hydrazine (1) is reacted with β-ketoester (2) in the presence of a suitable solvent, such as acetic acid and a base such as sodium acetate to provide pyrazole (3). The reaction mixture is heated to a temperature ranging from 80° C. to about 120° C., preferably about 100° C., for a period ranging from about 8 to about 24 hours, preferably about 16 hours or until analysis indicates the reaction is complete. The reaction mixture is then cooled to about 25° C. and the suitable acid is removed. Suitable solvent such as ethyl acetate and water are added and the organic layer is separated, dried, and concentrated to provide a pyrazole of structure (3) which can then be purified by standard, well known techniques.

An appropriate phenyl or pyridyl hydrazine (1) is one in which R⁵, R⁶, R⁷, and X⁷ are as desired in the final product of formula (I). Many of the phenyl or pyridyl hydrazines of structure (1) are commercially available such as phenylhydrazine hydrochloride, o-tolylhydrazine hydrochloride, m-tolylhydrazine hydrochloride, p-tolylhydrazine hydrochloride, 2-hydrazinopyridine dihydrochloride, 2-fluorophenylhydrazine hydrochloride, 3-fluorophenylhydrazine hydrochloride, 4-fluorophenylhydrazine hydrochloride, 2,4-difluorophenylhydrazine hydrochloride, 2,3-dichlorophenylhydrazine hydrochloride and the like or can be synthesized by methods well known and appreciated by those of ordinary skill in the art. For example, the procedure of Asselin, et. al (Asselin, A. A.; Humber, L. G.; Dobson, T. A.; Komlossy, J.; Martel, R. R. J. Med. Chem. 1976, 19, 787-792) describes a general method for the preparation of substituted phenyl hydrazines and their salts from readily available substituted anilines.

An appropriate β-ketoester (2) is one in which R⁷ is as desired in the final product of formula (I). Many of the β-ketoesters of structure (2) are commercially available such as ethyl acetoacetate, 4-methyl-3-oxo-pentanoic acid methyl ester, 3-oxo-pentanoic acid methyl ester and the like or can be synthesized by methods well known and appreciated by those of ordinary skill in the art as depicted in Scheme A1. For example, the reaction of suitable methyl ketones of structure (2a) with dialkyl carbonates such as dimethyl carbonate (R^{P1}=Me), diethyl carbonate (R_{P1}=Et) or the like in the presence of a suitable base such as sodium methoxide in a solvent such as methanol or the like will provide β-ketoesters of structure (2) that can be utilized in Scheme A.

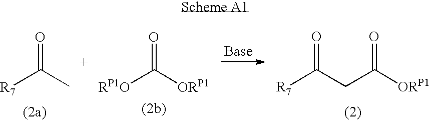

In Scheme A, step 2, pyrazole (3) is reacted with the 2-halo-1-nitrobenzene or heteroaryl analog of structure (4) in the presence of a base to provide the intermediate of structure (5). The reaction mixture is heated to a temperature ranging from 50° C. to about 100° C., preferably about 70° C., for a period ranging from about 8 to about 24 hours, preferably about 16 hours or until analysis indicates the reaction is complete. The reaction mixture is then cooled to about 25° C. and suitable solvent such as ethyl acetate and water are added. The resulting intermediate of structure (5) can be isolated and purified by techniques well known in the art such as extraction, evaporation, trituration, chromatography, and the like.

An appropriate 2-halo-1-nitrobenzene or heteroaryl analog of structure (4) is one in which X¹, X², X³, X⁴, R⁸, and R⁹ are as desired in the final product of formula (I). Many of the phenyl or 2-halo-1-nitrobenzene or heteroaryl analogs of structure (4) are commercially available such as 2-fluoronitrobenzene, 2-chloro-3-nitropyridine, 4-chloro-3-nitropyridine, 2,3-dichloronitrobenzene, 2-chloro-5 methyl-3-nitrobenzene, 2,3-difluoronitrobenzene, 2,5-difluoronitrobenzene, 3-chloro-2-fluoronitrobenzene, 2-fluoro-6-methoxynitrobenzene, 5-cyano-2-fluoronitrobenzene and the like or can be synthesized by methods well known and appreciated by those of ordinary skill in the art. For example, phenyl or 2-halo-1-nitrobenzene or heteroaryl analogs of structure (4) can be readily prepared by aromatic nitration of haloaromatics as described by Dal, E. et. al. (Dal, E.; Lancaster, N. L. Organic & Biomolecular Chemistry 2005, 3, 682-686).

In Scheme A, step 3, the nitro moiety in the intermediate of structure (5) is converted to an amine by catalytic hydrogenation to provide the amine of structure (6). For example, the intermediate of structure (5) is contacted with a suitable palladium species such as 5% or 10% carbon on palladium, in a suitable solvent or solvent mixture, such as methanol, under an inert atmosphere, such as nitrogen. The reaction vessel is then evacuated, flushed with nitrogen and filled with hydrogen, typically via balloon. The reaction mixture is then stirred for a period of time ranging from about 0.5 hours to about 6 hours, preferably for about 1.5 hours at approximately ambient temperature, preferably about 25° C. The reaction vessel is then flushed with nitrogen and filtered. The filtrate is then concentrated and purified by standard techniques such as chromatography to provide the amine of structure (6).

In Scheme A, step 4, the amine of structure (6) is coupled with the phenyl isocyanate of structure (7) to provide the N-phenyl pyrazole derivative of structure (8). For example, the amine of structure (6) is contacted with an appropriate phenyl isocyanate of structure (7) in the presence of a suitable solvent such as tetrahydrofuran, pyridine, acetonitrile, toluene, or dimethylformamide. The reaction carried out in the presence of from 1.0 to 6.0 molar equivalents of a suitable base such as sodium carbonate, sodium bicarbonate, potassium carbonate, potassium bicarbonate, triethylamine, pyridine, or diisopropylethylamine. The reaction is generally carried out from ambient temperature to refluxing temperature of the solvent. Generally the reaction takes from about 1 to 72 hours. The N-phenyl pyrazole derivative of structure (8) can be isolated and purified by techniques well known in the art such as extraction, evaporation, trituration, chromatography, and the like.

An appropriate phenyl isocyanate of structure (7) is one in which R¹, R², R³, X¹, X⁵ and X⁶ are as desired in the final product of formula (I). Many of the phenyl isocyanates of structure (7) are commercially available such as 4-chlorophenyl isocyanate, 4-tert-butylphenyl isocyanate, 3-chlorophenyl isocyanate, 2-chlorophenyl isocyanate, 4-fluorophenyl isocyanate, 4-methoxyphenyl isocyanate, 4-(trifluoromethyl)phenyl isocyanate, 4-(trifluoromethoxy)phenyl isocyanate, 4-iso-propylphenyl isocyanate, and the like or can be synthesized by methods well known and appreciated by those of ordinary skill in the art. For example, substituted anlines of can be treated with phosgene, diphosgene, triphosgene or the like in the presence of a base such as 1,8-bis(dimethylamino)naphthalene, triethylamine, pyridine or the like in a solvent such as dichloromethane, chloroform, THF, acetonitrile or the like. The reaction mixture is stirred at a temperature range of heated to a temperature ranging from −10° C. to about 10° C., preferably about 0° C., for a period ranging from about 1 to about 5 hours, preferably about 2 hours or until analysis indicates the reaction is complete. Using standard work up procedures the reaction mixture is washed, dried and concentrated to provide isocyanate of structure (7).

For compounds of formula (I) where Y is thio, as illustrated below in Scheme A2, one can treat structure (3) with Lawesson's reagent ([2,4-bis(4-methoxyphenyl)-1,3-diathia-2,4-diphosphetane-2,4-disulfide) at elevated temperatures in a suitable solvent to prepare thiopyrazoles of general structure (3a). These thiopyrazoles (3a) can be substituted for the pyrazole of general structure (3) in Scheme A to allow for preparation of compounds of formula (I) where Y is thio.

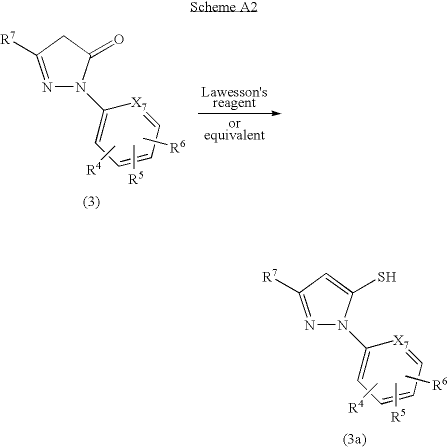

Compounds of formula (I) wherein R⁷ occupies the 4-position of the pyrazole can be prepared using a variation of the route illustrated in Scheme A. In this variation, as shown in scheme A3 below, substituted phenyl or pyridyl hydrazine hydrochlorides of structure (1) can be condensed with substituted enol esters of general structure (2c) in the presence of a suitable base to afford pyrazoles of structure (3b). These pyrazoles of structure (3b) can then be substituted for pyrazoles of structure (3) in Scheme A to enable the synthesis of examples of formula (I) wherein R⁷ occupies the 4-position of the pyrazole.

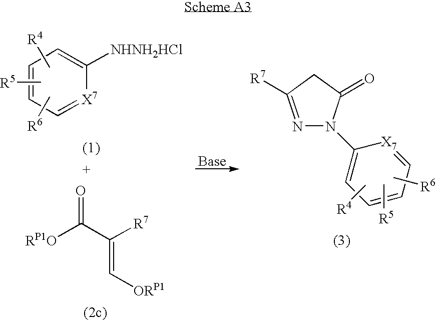

Compounds of general structure (8) can also be prepared by an alternative method as outlined in Scheme B. In this method amine (6) is prepared as previously described in Scheme 1. Subsequent treatment of amine (6) with phosgene or a suitable equivalent in the presence of a base affords isocyanate (9) which can then be reacted with aniline (10) to afford compound (8) as illustrated.

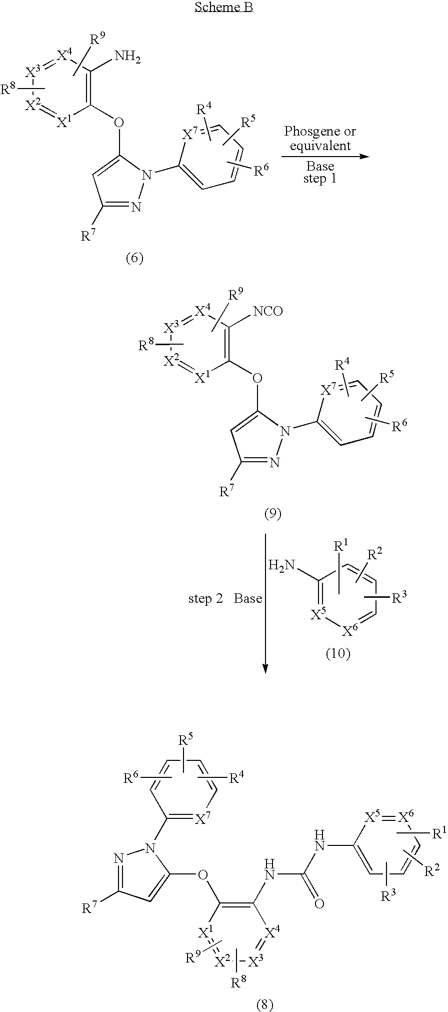

In Scheme B, step 1, anlines of structure (6) can be converted to isocyanates of structure (9) by treatment with phosgene or its equivalent and a suitable base in a suitable solvent. For example, anilines of structure (6) can be treated with phosgene, diphosgene, triphosgene or the like in the presence of a base such as 1,8-bis(dimethylamino)naphthalene, triethylamine, pyridine or the like in a solvent such as dichloromethane, chloroform, THF, acetonitrile or the like. The reaction mixture is stirred at a temperature range of heated to a temperature ranging from −10° C. to about 10° C., preferably about 0° C., for a period ranging from about 1 to about 5 hours, preferably about 2 hours or until analysis indicates the reaction is complete. Using standard work up procedures the reaction mixture is washed dried and concentrated to provide isocyanate of structure (9).

In Scheme B, step 2, the isocyanate of structure (9) is contacted with an appropriate aniline of structure (10) under conditions described in Scheme A, step 4 to provide N-phenyl pyrazole derivative of structure (8)

An appropriate phenyl aniline of structure (10) is one in which R¹, R², R³, X⁴, X⁵, and X⁶ are as desired in the final product of formula (I). Many of the anilines of structure (10) are commercially available such as 4-tert-butylaniline, 4-chloroaniline, 4-(trifluoromethoxy)aniline, 4-cyclohexylaniline, 2-fluoro-4-(trifluoromethyl) anline and the like or can be synthesized by methods well known and appreciated by those of ordinary skill in the art. For example, catalytic hydrogenation of substituted nitroaromatic compounds will provide substituted anilines of structure (10).

The following non-limiting Preparations and Examples illustrate the preparation of compounds of the present invention.

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCI). The following abbreviations have been used for common solvents: CDCl₃, deuterochloroform; D₆-DMSO, deuterodimethylsulphoxide; CD₃OD, deuteromethanol; THF, tetrahydrofuran. ‘Ammonia’ refers to a concentrated solution of ammonia in water possessing a specific gravity of 0.88. Where thin layer chromatography (TLC) has been used it refers to silica gel TLC using silica gel 60 F₂₅₄ plates, R_{f} is the distance traveled by a compound divided by the distance traveled by the solvent front on a TLC plate.

### EXAMPLE 1

### 1-[2-(5-Methyl-2-o-tolyl-2H-pyrazol-3-yloxy)-phenyl]-3-(4-trifluoromethoxy-phenyl)-urea

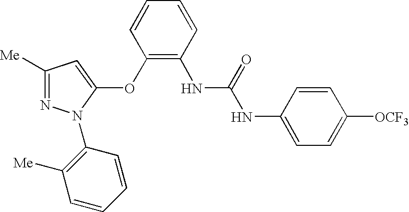

### Step A

### 5-methyl-2-o-tolyl-2H-pyrazol-3-ol

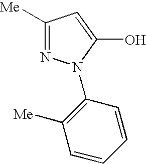

A solution of o-tolyl-hydrazine hydrochloride (10.0 g, 63.0 mmol), sodium acetate (5.7 g, 69.3 mmol) and ethyl acetoacetate (8.2 g, 63.0 mmol) in glacial acetic acid (100 mL) was heated to 100° C. for 16 hrs. The reaction mixture was then cooled to 25° C. and the acetic acid was removed under reduced pressure. Ethyl acetate (150 mL) and water (150 mL) were added and the organic layer was separated, dried over Na₂SO₄ and concentrated to a brown solid. This solid was washed with ethyl acetate and isolated by filtration to afford 5-methyl-2-o-tolyl-2,4-dihydro-pyrazol-3-one (6.9 g, 58%). ¹H-NMR (400 MHz, d-DMSO) δ 7.29-7.15 (m, 4H), 5.29 (s, 1H), 2.08 (s, 3H), 2.06 (s, 3H).

### Step B

### 3-Methyl-5-(2-nitro-phenoxy)-1-o-tolyl-1H-pyrazole

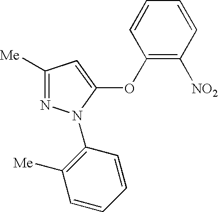

To a solution of 1-fluoro-2-nitrobenzene (5.2 g, 36.7 mmol) and 5-methyl-2-o-tolyl-2H-pyrazol-3-ol (6.9 g, 36.7 mmol) in DMF (60 mL) was added potassium carbonate (5.6 g, 40.3 mmol), and the reaction mixture was heated to 70° C. for 16 hrs. The reaction mixture was then cooled to 25° C. and water (100 mL) and ethyl acetate (100 mL) were added. The organic layer was separated, washed with brine, dried over Na₂SO₄ and concentrated to a brown oil that was purified by column chromatography (10-20% ethyl acetate/hexane) to provide 3-methyl-5-(2-nitro-phenoxy)-1-o-tolyl-1H-pyrazole (4.45 g, 39%). ¹H-NMR (400 MHz, d-CDCl₃) δ 7.86 (dd, 1H), 7.52 (t, 1H), 7.29-7.17 (m, 5H), 5.60 (s, 1H), 2.28 (s, 3H), 2.22 (s, 3H).

### Step C

### 2-(5-Methyl-2-o-tolyl-2H-pyrazol-3-yloxy)-phenylamine

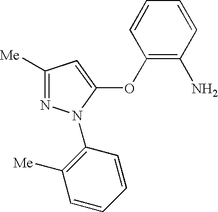

To a solution of 3-methyl-5-(2-nitro-phenoxy)-1-o-tolyl-1H-pyrazole (4.34 g, 14.0 mmol) in MeOH (100 mL) under a nitrogen atmosphere was added 10% Pd/C (200 mg). The reaction vessel was evacuated, flushed with nitrogen and then filled with hydrogen via balloon. The reaction mixture was stirred at 25° C. for 1.5 hrs after which time the reaction vessel was flushed with nitrogen and filtered through a pad of celite. The filtrate was concentrated to an oil that was purified by column chromatography (15-20% ethyl acetate/hexane) to provide 2-(5-methyl-2-o-tolyl-2H-pyrazol-3-yloxy)-phenylamine (2.69 69%). ¹H-NMR (400 MHz, d-CDCl₃) δ 7.33-7.21 (m, 4H), 7.03 (dd, 1H), 6.95 (t, 1H), 6.74-6.66 (m, 2H), 5.47 (s, 1H), 3.59 (bs, 2H), 2.25 (s, 3H), 2.24 (s, 3H).

### Step D

### 1-[2-(5-Methyl-2-o-tolyl-2H-pyrazol-3-yloxy)-phenyl]-3-(4-trifluoromethoxy-phenyl)-urea

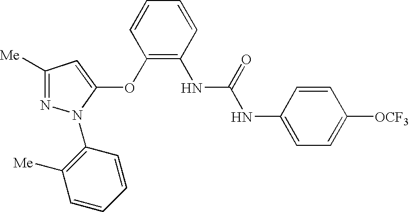

To a solution of 2-(5-methyl-2-o-tolyl-2H-pyrazol-3-yloxy)-phenylamine (0.47 g, 1.70 mmol) in THF (15 mL) was added 4-(trifluoromethoxy)-phenyl isocyanate (0.34 g, 1.70 mmol) and triethyl amine (0.26 g, 2.55 mmol). The reaction mixture was stirred at 65° C. for 4 hrs and then cooled to 25° C. The solvent was removed under reduced pressure and the resulting residue was purified by column chromatography (15-25% ethyl acetate/hexane) to afford 1-[2-(5-methyl-2-o-tolyl-2H-pyrazol-3-yloxy)-phenyl]-3-(4-trifluoromethoxy-phenyl)-urea (0.45 g, 55%). ¹H-NMR (400 MHz, d-DMSO) δ 9.31 (s, 1H), 8.23 (s, 1H), 8.01 (dd, 1H), 7.51-7.47 (m, 2H), 7.33 (dd, 1H), 7.29-7.17 (m, 5H), 7.07-6.91 (m, 3H), 5.72 (s, 1H), 2.16 (s, 3H), 2.14 (s, 3H).

### EXAMPLE 2

### 1-{2-[2-(2-Chloro-phenyl)-5-methyl-2H-pyrazol-3-yloxy]-phenyl}-3-(4-trifluoromethoxy-phenyl)-urea

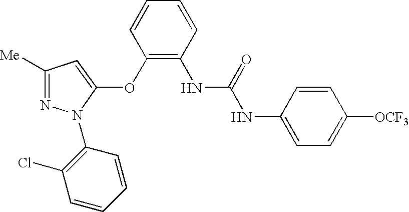

### Step A

### 1-(2-Chloro-phenyl)-3-methyl-5-(2-nitro-phenoxy)-1H-pyrazole

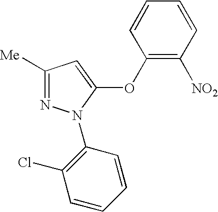

To a solution of 1-fluoro-2-nitrobenzene (5.0 g, 35.4 mmol) and 2-(2-chloro-phenyl)-5-methyl-2H-pyrazol-3-ol (9.6 g, 46.1 mmol) in DMF (100 mL) was added potassium carbonate (12.2 g, 88.6 mmol), and the reaction mixture was heated to 70° C. for 16 hrs. The reaction mixture was then cooled to 25° C. and water (100 ml), diethyl ether (200 ml) and ethyl acetate (100 ml) were added. The organic layer was separated, washed with brine, dried over Na₂SO₄ and concentrated to a brown oil that was purified by column chromatography (20-30% ethyl acetate/hexane) to provide 1-(2-chloro-phenyl)-3-methyl-5-(2-nitro-phenoxy)-1H-pyrazole (4.49 g, 39%). ¹H-NMR (400 MHz, d-CDCl₃) δ 7.87 (dd, 1H), 7.57-7.58 (m, 1H), 7.55-7.21 (m, 6H), 5.58 (s, 1H), 2.28 (s, 3H).

### Step B

### 2-[2-(2-Chloro-phenyl)-5-methyl-2H-pyrazol-3-yloxy]-phenylamine

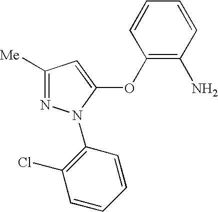

To 1-(2-chloro-phenyl)-3-methyl-5-(2-nitro-phenoxy)-1H-pyrazole (1.0 g, 3.0 mmol) in EtOH (30 ml) was added water (10 ml) followed by iron dust (0.50 g, 9.1 mmol) and ammonium chloride (0.195 g. 3.6 mmol). The reaction mixture was heated at 80° C. for 2 hrs. The reaction mixture was then cooled to 25° C. and filtered through celite. The solid residue was washed with EtOH (50 ml). The filtrate was then concentrated and the resulting residue was dissolved in ethyl acetate (50 ml), washed with water (20 ml), and dried over anhydrous sodium sulfate. The crude product was purified by silica gel column chromatography (35% ethyl acetate/hexane) to afford 2-[2-(2-chloro-phenyl)-5-methyl-2H-pyrazol-3-yloxy]-phenylamine (0.88 g, 96%). ¹H-NMR (400 MHz, d-DMSO) δ 7.64-7.59 (m, 2H), 7.50-7.44 (m, 2H), 6.93-6.85 (m, 2H), 6.71 (dd, 1H), 6.52-6.47 (m, 1H), 5.35 (s, 1H), 4.833 (bs, 2H), 2.10 (s, 3H).

### Step C

### 1-{2-[2-(2-Chloro-phenyl)-5-methyl-2H-pyrazol-3-yloxy]-phenyl}-3-(4-trifluoromethoxy-phenyl)-urea

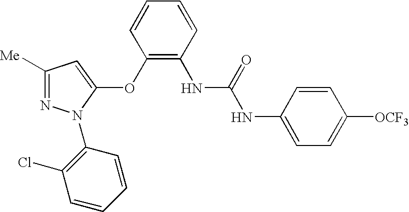

To a solution of 2-[2-(2-chloro-phenyl)-5-methyl-2H-pyrazol-3-yloxy]-phenylamine (0.35 g, 1.17 mmol) in DMF (3 mL) was added 4-(trifluoromethoxy)-phenyl isocyanate (0.25 g, 1.23 mmol) in DMF (1 ml) and triethyl amine (0.26 g, 2.55 mmol). The reaction mixture was stirred at 75° C. for 4 hrs and then cooled to 25° C. The reaction mixture was loaded onto silica gel column and purified (15-25% ethyl acetate/hexane) to afford 1-{2-[2-(2-chloro-phenyl)-5-methyl-2H-pyrazol-3-yloxy]-phenyl}-3-(4-trifluoromethoxy-phenyl)-urea (0.28 g, 47%). ¹H-NMR (400 MHz, d-CDCl₃) δ 8.10 (dd, 1H), 7.47-7.41 (m, 2H), 7.34-7.25 (m, 3H), 7.23-6.98 (m, 6H), 5.49 (s, 1H), 2.25 (s, 3H).

### EXAMPLES 3-20

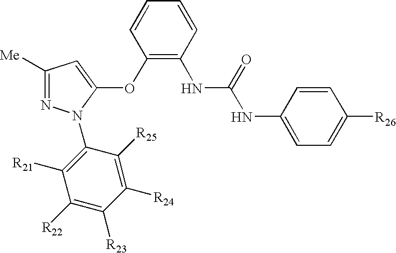

Examples 3-20 were prepared according to the methods of Example 1 and Example 2 using the appropriate commercially available substituted phenyl hydrazine starting materials.

| Example | R₂₁ | R₂₂ | R₂₃ | R₂₄ | R₂₅ | R₂₆ | H-NMR |
|---|---|---|---|---|---|---|---|
| 3 | Et | H | H | H | H | OCF₃ | ¹H-NMR (400 MHz, d-CDCl3) δ 8.02 (d, 1 |
| | | | | | | | H), 7.31-7.06 (m, 11 H), 6.88 (bs, 1 H), |
| | | | | | | | 5.55 (s, 1 H), 2.45 (q, 2 H), 2.21 (s, 3 H), |
| | | | | | | | 1.08 (t, 3 H) |
| 4 | Cl | Cl | H | H | H | OCF₃ | ¹H-NMR (400 MHz, d-CDCl3) δ 8.11 (d, 1 |
| | | | | | | | H), 7.50 (d, 1 H), 7.37-7.31 (m, 3 H), 7.22- |
| | | | | | | | 6.98 (m, 6 H), 5.53 (s, 1 H), 2.25 (s, 3 H). |
| 5 | H | Me | H | H | H | OCF₃ | ¹H-NMR (400 MHz, d-CDCl3) δ 8.38 (s, 1 |
| | | | | | | | H), 8.31 (dd, 1 H), 7.63 (s, 1 H), 7.36-7.31 |
| | | | | | | | (m, 4 H), 7.29-7.13 (m, 2 H), 7.10-6.97 |
| | | | | | | | (m, 5 H), 5.33 (s, 1 H), 2.18 (s, 3 H), 1.55 |
| | | | | | | | (s, 3 H). |
| 6 | Cl | H | H | H | H | t-Bu | ¹H-NMR (400 MHz, d-DMSO) δ 9.02 (s, 1 |
| | | | | | | | H), 8.13 (s, 1 H), 8.05 (d, 1 H), 7.59-7.54 |
| | | | | | | | (m, 2 H), 7.44-7.40 (m, 2 H), 7.31-7.24 (m, |
| | | | | | | | 4 H), 7.06-7.04 (m, 2 H), 6.95-6.92 (m, 1 |
| | | | | | | | H), 5.67 (s, 1 H), 2.15 (s, 3 H), 1.22 (s, 9 |
| | | | | | | | H). |
| 7 | H | Cl | H | H | H | OCF₃ | ¹H-NMR (400 MHz, d-DMSO) δ 9.27 (s, 1 |
| | | | | | | | H), 8.51 (s, 1 H), 8.15 (dd, 1 H), 7.79-7.78 |
| | | | | | | | (m, 1 H), 7.72-7.69 (m, 1 H), 7.53-7.43 (m, |
| | | | | | | | 4 H), 7.33 (dd, 1 H), 7.26-7.24 (d, 1 H), |
| | | | | | | | 7.18-7.08 (m, 2 H), 6.99-6.95 (m, 1 H), |
| | | | | | | | 5.64 (s, 1 H), 2.15 (s, 3 H). |
| 8 | CF₃ | H | H | H | H | OCF₃ | ¹H-NMR (400 MHz, d-DMSO) δ 9.26 (s, 1 |
| | | | | | | | H), 8.17 (s, 1 H), 7.96 (d, 1 H), 7.85 (d, 1 |
| | | | | | | | H), 7.72-7.63 (m, 3 H), 7.48 (d, 2 H), 7.25 |
| | | | | | | | (d, 2 H), 7.09-7.06 (m, 1 H), 6.98-6.96 (m, |
| | | | | | | | 2 H), 5.66 (s, 1 H), 2.13 (s, 3 H) |
| 9 | F | H | H | H | H | OCF₃ | ¹H-NMR (400 MHz, d-DMSO) δ 9.28 (s, 1 |
| | | | | | | | H), 8.26 (s, 1 H), 8.09 (d, 1 H), 7.58 (t, 1 |
| | | | | | | | H), 7.56-7.23 (m, 7 H), 7.11-6.79 (m, 3 H), |
| | | | | | | | 5.69 (s, 1 H), 2.15 (s, 3 H) |
| 10 | H | H | Me | H | H | OCF₃ | ¹H-NMR (400 MHz, d-DMSO) δ 9.33 (s, 1 |
| | | | | | | | H), 8.48 (s, 1 H), 8.15 (d, 1 H), 7.56-7.49 |
| | | | | | | | (m, 4 H), 7.26-7.20 (m, 4 H), 7.10 (t, 1 H), |
| | | | | | | | 7.00-6.92 (m, 2 H), 5.66 (s, 1 H), 2.25 (s, |
| | | | | | | | 3 H), 2.15 (s, 3 H). |
| 11 | OMe | H | H | H | H | OCF₃ | ¹H-NMR (400 MHz, d-CDCl3) δ 8.25 (d, 1 |
| | | | | | | | H), 7.83 (s, 1 H), 7.32-7.16 (m, 5 H), 7.03- |
| | | | | | | | 6.97 (m, 3 H), 6.88-6.83 (m, 2 H), 5.35 (s, |
| | | | | | | | 1 H), 3.40 (s, 3 H), 2.17 (s, 3 H). |
| 12 | H | H | H | H | H | tBu | ¹H-NMR (400 MHz, d-CDCl3) δ 8.32 (dd, |
| | | | | | | | 1 H), 7.55-7.52 (m, 2 H), 7.32-7.28 (m, 2 |
| | | | | | | | H), 7.24-7.16 (m, 7 H), 7.07-7.04 (m, 1 H) |
| | | | | | | | 6.99-6.95 (m, 1 H), 5.38 (s, 1 H), 2.20 (s, |
| | | | | | | | 3 H), 1.23 (s, 9 H). |
| 13 | H | H | Cl | H | H | OCF₃ | ¹H-NMR (400 MHz, d-DMSO) δ 9.27 (s, 1 |
| | | | | | | | H), 8.48 (s, 1 H), 8.15 (dd, 1 H), 7.76-7.72 |
| | | | | | | | (m, 2 H), 7.52-7.47 (m, 4 H), 7.26 (s, 1 H), |
| | | | | | | | 7.24 (s, 1 H), 7.17-7.13 (m, 1 H), 7.10- |
| | | | | | | | 7.07 (m, 1 H), 7.00-6.96 (m, 1 H), 5.61 (s, |
| | | | | | | | 1 H), 2.15 (s, 3 H). |
| 14 | F | H | F | H | H | OCF₃ | ¹H-NMR (400 MHz, d-DMSO) δ 9.26 (s, 1 |
| | | | | | | | H), 8.31 (s, 1 H), 8.07 (d, 1 H), 7.64-7.62 |
| | | | | | | | (m, 1 H), 7.49-7.43 (m, 3 H), 7.24 (d, 2 H), |
| | | | | | | | 7.23-6.96 (m, 5 H), 5.70 (s, 1 H), 2.15 (s, |
| | | | | | | | 3 H). |
| 15 | H | H | F | H | H | OCF₃ | ¹H-NMR (400 MHz, d-DMSO) δ 9.29 (s, 1 |
| | | | | | | | H), 8.48 (s, 1 H), 8.16 (d, 1 H), 7.74-7.72 |
| | | | | | | | (m, 2 H), 7.54 (d, 2 H), 7.54 (d, 2 H), 7.30- |
| | | | | | | | 6.94 (m, 7 H), 5.62 (s, 1 H), 2.15 (s, 3 H) |
| 16 | H | H | H | H | H | OCF₃ | ¹H-NMR (400 MHz, d-CDCl3) δ 8.31 (dd, |
| | | | | | | | 1 H), 8.24 (s, 1 H), 7.59 (m, 1 H), 7.32- |
| | | | | | | | 7.16 (m, 5 H), 7.03-6.97 (m, 3 H), 6.88- |
| | | | | | | | 6.83 (m, 2 H), 5.35 (s, 1 H), 3.40 (s, 3 H), |
| | | | | | | | 2.17 (s, 3 H). |
| 17 | H | F | H | H | H | OCF₃ | ¹H-NMR (400 MHz, d-DMSO) δ 9.28 (s, 1 |
| | | | | | | | H), 8.51 (s, 1 H), 8.16 (d, 1 H), 7.61-7.44 |
| | | | | | | | (m, 4 H), 7.25 (d, 2 H), 7.17-6.90 (m, 4 H), |
| | | | | | | | 5.62 (s, 1 H), 2.15 (s, 3 H). |
| 18 | H | H | OMe | H | H | OCF₃ | ¹H-NMR (400 MHz, d-DMSO) δ 9.33 (s, 1 |
| | | | | | | | H), 8.47 (s, 1 H), 8.14 d, 1 H), 7.58-7.49 |
| | | | | | | | (m, 4 H), 7.25 (d, 2 H), 7.09 (t, 1 H), 6.98- |
| | | | | | | | 6.92 (m, 4 H), 5.65 (s, 1 H), 3.70 (s, 3 H), |
| | | | | | | | 2.15 (s, 3 H). |
| 19 | Cl | H | H | H | Cl | OCF₃ | ¹H-NMR (400 MHz, d-DMSO) δ 9.30 (s, 1 |
| | | | | | | | H), 8.02 (s, 1 H), 7.88 (d, 1 H), 7.70 (dd, 2 |
| | | | | | | | H), 7.49-7.45 (m, 3 H), 7.26-7.23 (m, 2 H), |
| | | | | | | | 7.11-7.00 (m, 2 H), 6.99-6.96 (m, 1 H), |
| | | | | | | | 5.75 (s, 1 H), 2.16 (s, 3 H). |
| 20 | Cl | H | Cl | H | H | OCF₃ | ¹H-NMR (400 MHz, d-DMSO) δ 9.24 (s, 1 |
| | | | | | | | H), 8.21 (s, 1 H), 8.01 (d, 1 H), 7.75 (d, 1 |
| | | | | | | | H), 7.57-7.46 (m, 4 H), 7.26 (d, 2 H), 7.11- |
| | | | | | | | 7.07 (m, 2 H), 6.98-6.94 (m, 1 H), 5.70 (s, |
| | | | | | | | 1 H), 2.15 (s, 3 H) |

### EXAMPLE 21

### 1-(4-tert-Butyl-phenyl)-3-[2-(5-methyl-2-phenyl-2H-pyrazol-3-yloxy)-pyridin-3-yl]-urea

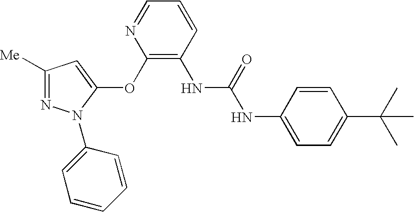

### Step A

### 2-(5-Methyl-2-phenyl-2H-pyrazol-3-yloxy)-3-nitro-pyridine

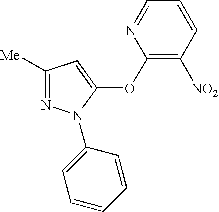

To a solution of 2-chloro-3-nitro-pyridine (1.09 g, 6.88 mmol) and 5-methyl-2-phenyl-2H-pyrazol-3-ol (1.20 g, 6.88 mmol) in dimethyl formamide (7 mL) was added cesium carbonate (3.4 g, 10.4 mmol). The reaction mixture was stirred at 80° C. for 12 hours. The mixture was cooled to 25° C., diluted with ethyl acetate (150 mL) and filtered through a pad of celite. The filtrate was washed with 1 N HCl, dried over Na₂SO₄, filtered and concentrated in vacuo. The crude product was purified by column chromatography (0-5% MeOH/CH₂Cl₂) to yield desired 2-(5-methyl-2-phenyl-2H-pyrazol-3-yloxy)-3-nitro-pyridine (0.47 g, 23%). ¹H-NMR (400 MHz, d-CDCl₃) δ 8.33 (dd, 1H), 8.32 (s, 1H), 7.65-7.62 (m, 2H), 7.38-7.33 (m, 2H), 7.24-7.17 (m, 3H), 6.08 (s, 1H), 2.36 (s, 3H).

### Step B

### 2-(5-Methyl-2-phenyl-2H-pyrazol-3-yloxy)-pyridin-3-ylamine

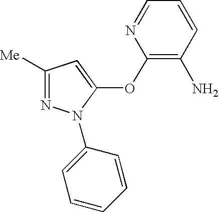

2-(5-Methyl-2-phenyl-2H-pyrazol-3-yloxy)-3-nitro-pyridine (0.47 g, 1.59 mmol) in the mixture of tetrahydrofuran and MeOH (1:1, 25 ml) was treated with 10% Pd on carbon 0.20 g). The reaction mixture was stirred at 25° C. under hydrogen atmosphere for 1 hr. The catalyst was filtered off and the filtrate was concentrated to afford 2-(5-methyl-2-phenyl-2H-pyrazol-3-yloxy)-pyridin-3-ylamine (0.41, 96%) ¹H-NMR (400 MHz, d-CDCl₃) δ 7.57-7.51 (m, 4H), 7.39-7.32 (m, 3H), 7.24-7.15 (m, 1H), 7.00-6.98 (m, 1H), 6.87-6.83 (m, 1H), 5.97 (s, 1H), 2.27 (s, 3H).

### Step C

### 1-(4-tert-Butyl-phenyl)-3-[2-(5-methyl-2-phenyl-2H-pyrazol-3-yloxy)-pyridin-3-yl]-urea

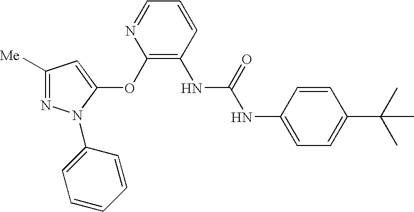

To a solution of 2-(5-methyl-2-phenyl-2H-pyrazol-3-yloxy)-pyridin-3-ylamine (0.15 g, 0.56 mmol) in 1,4-dioxane (5 mL) was added a solution of 1-tert-butyl-4-isocyanato-benzene (0.14 g, 0.77 mmol) in 1,4-dioxane (2 mL). The reaction was stirred at 80° C. for 12 hrs. The reaction was concentrated in vacuo. The crude product was purified by column chromatography (0-1.5% MeOH/CHCl₃) to afford 1-(4-tert-butyl-phenyl)-3-[2-(5-methyl-2-phenyl-2H-pyrazol-3-yloxy)-pyridin-3-yl]-urea (0.14 g, 55%). ¹H-NMR (400 MHz, d-CDCl₃) δ 8.71 (dd, 1H), 7.78 (dd, 4H), 7.39-7.32 (m, 3H), 7.24-7.15 (m, 1H), 7.00-6.98 (m, 1H), 6.87-6.83 (m, 1H), 5.97 (s, 1H), 2.27 (s, 3H) 1.26 (s, 9H).

### EXAMPLE 22

### 1-{2-[2-(2-Chloro-phenyl)-5-methyl-2H-pyrazol-3-yloxy]-pyridin-3-yl}-3-(4-trifluoromethoxy-phenyl)-urea

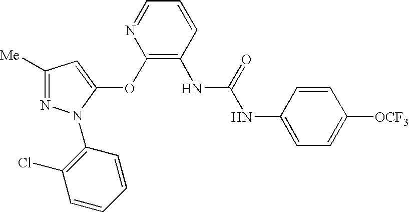

### Step A

### 2-[2-(2-Chloro-phenyl)-5-methyl-2H-pyrazol-3-yloxy]-3-nitro-pyridine

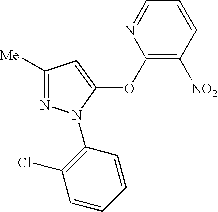

A solution of 2-chloro3-nitropyridine (4.00 g, 25.2 mmol) in DMF (150 ml) was treated with 2-(2-chloro-phenyl)-5-methyl-2H-pyrazol-3-ol (6.32 g, 30.28 mmol) and potassium carbonate (8.72 g, 63.1 mmol). The mixture was stirred at 80° C. for 12 hours. The reaction mixture was cooled to 25° C. and water (100 mL) was added and the mixture was extracted with diethyl ether (300 ml) and ethyl acetate (100 ml). The combined organic phases were washed with water (50 ml) and brine (50 mL), dried over Na₂SO₄, filtered and concentrated. The crude product was purified by column chromatography (20-35% ethyl acetate/heptane) to yield desired 2-[2-(2-chloro-phenyl)-5-methyl-2H-pyrazol-3-yloxy]-3-nitro-pyridine (4.56 g, 55%). ¹H-NMR (400 MHz, d-CDCl₃) δ 8.40 (dd, 1H), 8.27 (dd, 1H), 7.46-7.41 (m, 2H), 7.38-7.27 (m, 2H), 7.24-7.18 (m, 1H), 6.20 (d, 1H), 2.37 (s, 3H).

### Step B

### 2-[2-(2-Chloro-phenyl)-5-methyl-2H-pyrazol-3-yloxy]-pyridin-3-ylamine

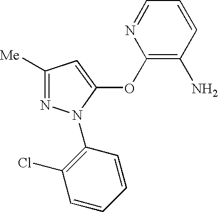

The 2-[2-(2-chloro-phenyl)-5-methyl-2H-pyrazol-3-yloxy]-3-nitro-pyridine (1.65 g, 5.00 mmol) dissolved in the mixture of water and EtOH (1: 2.5, 70 ml) was treated with iron dust (0.84 g, 15.0 mmol) and ammonium chloride (0.32 g, 6.0 mmol). The reaction was heated to 80° C. for 2 hrs. The reaction mixture was then cooled to 25° C. and filtered through celite. The filtrate was concentrated and dissolved in ethyl acetate (50 ml), washed with water (20 ml), dried over Na₂SO₄, filtered and concentrated. The crude product was purified by column chromatography (35% ethyl acetate/heptane) to afford 2-[2-(2-chloro-phenyl)-5-methyl-2H-pyrazol-3-yloxy]-pyridin-3-ylamine (1.03. 69%). ¹H-NMR (400 MHz, d-DMSO) δ 7.58-7.56 (m, 1H), 7.47-7.34 (m, 3H) 7.28 (dd, 1H), 6.95 (dd, 1H), 6.86-6.82 (m, 1H), 5.90 (s, 1H), 4.95 (s, 2H), 2.17 (s, 3H).

### Step C

### 1-{2-[2-(2-Chloro-phenyl)-5-methyl-2H-pyrazol-3-yloxy]-pyridin-3-yl}-3-(4-trifluoromethoxy-phenyl)-urea

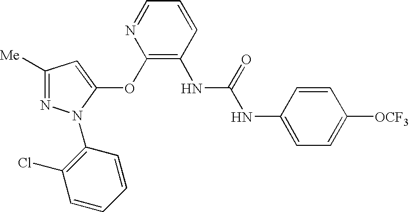

To 2-[2-(2-chloro-phenyl)-5-methyl-2H-pyrazol-3-yloxy]-pyridin-3-ylamine (1.03 g, 3.42 mmol) in DMF (10 ml), was added 4-(trifluoromethoxy)-phenyl isocyanate (0.73 g, 3.60 mmol) in DMF (2 ml), followed by triethyl amine (2 ml). The reaction mixture was heated at 75° C. for 18 hrs. After cooling to 25° C., the reaction mixture was loaded directly onto a silica gel column and purified by column chromatography (10-40%, ethyl acetate/heptane) to afford 1-{2-[2-(2-chloro-phenyl)-5-methyl-2H-pyrazol-3-yloxy]-pyridin-3-yl}-3-(4-trifluoromethoxy-phenyl)-urea (0.25 g, 15%). ¹H-NMR (400 MHz, d-DMSO) δ 9.41 (s, 1H), 8.40 (s, 1H), 8.35 (dd, 1H), 7.68-7.66 (m, 1H), 7.51-7.46 (m, 4H), 7.41-7.24 (m, 4H) 7.07-7.04 (m, 1H), 6.05 (s, 1H), 2.21 (s, 3H).

### EXAMPLE 23

### 1-[4-(5-Methyl-2-phenyl-2H-pyrazol-3-yloxy)-pyridin-3-yl]-3-(4-trifluoromethoxy-phenyl)-urea

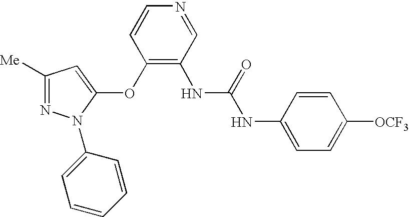

### Step A

### 4-(5-Methyl-2-phenyl-2H-pyrazol-3-yloxy)-3-nitro-pyridine

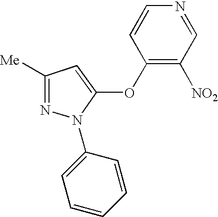

To a solution of 4-chloro-3-nitro-pyridine (4.5 g, 28.7 mmol) and 5-methyl-2-phenyl-2,4-dihydro-pyrazol-3-one (5.0 g, 28.7 mmol) in DMF (60 mL) was added potassium carbonate (4.4 g, 31.6 mmol), and the reaction mixture was stirred at 25° C. for 1 hr. Subsequently, water (100 mL) and ethyl acetate (100 mL) were added. The organic layer was separated, washed with brine, dried over Na₂SO₄ and concentrated to a brown oil that was purified by column chromatography (15-40% ethyl acetate/hexane) to provide 4-(5-methyl-2-phenyl-2H-pyrazol-3-yloxy)-3-nitro-pyridine (0.50 g, 6%). ¹H-NMR (400 MHz, d-CDCl₃) δ 9.08 (s, 1H), 8.55 (d, 1H), 7.58 (d, 2H), 7.39-7.34 (m, 2H), 7.26-7.22 (m, 1H), 6.98 (d, 1H), 5.93 (s, 1H), 2.34 (s, 3H).

### Step B

### 4-(5-Methyl-2-phenyl-2H-pyrazol-3-yloxy)-pyridin-3-ylamine

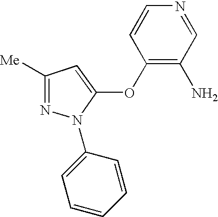

To a solution of 4-(5-methyl-2-phenyl-2H-pyrazol-3-yloxy)-3-nitro-pyridine (4.34 g, 14.0 mmol) in MeOH (50 mL) under a nitrogen atmosphere was added 10% Pd/C (10 mg). The reaction vessel was evacuated, flushed with nitrogen and then filled with hydrogen via balloon. The reaction mixture was stirred at 25° C. for 3 hrs after which time the reaction vessel was flushed with nitrogen and filtered through a pad of celite. The filtrate was concentrated to an oil that was purified by column chromatography (15-20% ethyl acetate/hexane) to provide 4-(5-methyl-2-phenyl-2H-pyrazol-3-yloxy)-pyridin-3-ylamine

(0.33 86%). ¹H-NMR (400 MHz, d-CDCl₃) δ 8.09 (s, 1H), 7.87 (d, 1H), 7.55-7.51 (m, 2H), 7.35-7.33 (m, 2H), 7.23-7.19 (m, 1H), 6.81 (d, 1H), 5.72 (s, 1H), 2.28 (3H).

### Step C

### 1-[4-(5-Methyl-2-phenyl-2H-pyrazol-3-yloxy)-pyridin-3-yl]-3-(4-trifluoromethoxy-phenyl)-urea

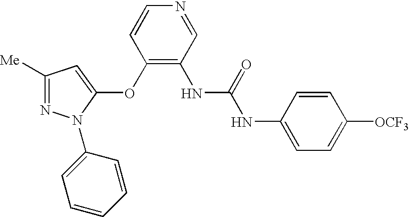

To a solution of 4-(5-methyl-2-phenyl-2H-pyrazol-3-yloxy)-pyridin-3-ylamine (0.32 g, 1.20 mmol) in THF (15 mL) was added 4-(trifluoromethoxy)-phenyl isocyanate (0.244 g, 1.20 mmol) and triethyl amine (0.18 g, 1.80 mmol). The reaction mixture was stirred at 65° C. for 6 hrs and then cooled to 25° C. The solvent was removed under reduced pressure and the resulting residue was purified by column chromatography (10-15% ethyl acetate/hexane) to afford 1-[4-(5-methyl-2-phenyl-2H-pyrazol-3-yloxy)-pyridin-3-yl]-3-(4-trifluoromethoxy-phenyl)-urea (0.42 g, 74%). ¹H-NMR (400 MHz, d-DMSO) δ 9.36 (s, 1H), 9.24 (s, 1H), 8.65 (s, 1H), 8.10 (d, 1H), 7.59-7.52 (m, 4H), 7.37 (t, 2H), 7.27-7.23 (m, 3H), 6.95 (d, 1H), 6.06 (s, 1H), 2.22 (s, 3H).

### EXAMPLE 24

### 1-[2-(2-Phenyl-2H-pyrazol-3-yloxy)-phenyl]-3-(4-trifluoromethoxy-phenyl)-urea

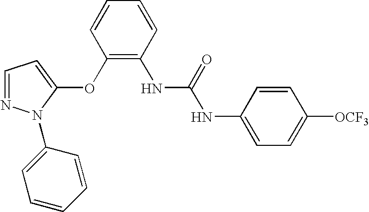

### Step A

### 3-Oxo-2-phenyl-2,3-dihydro-1H-pyrazole-4-carboxylic acid ethyl ester

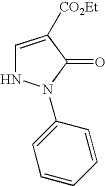

To a solution of phenyl hydrazine (10.0 g, 92.5 mmol) and K₂CO₃) 15.3 g, 111 mmol) in EtOH (100 mL) at 25° C. was added diethyl (ethoxymethlyene) malonate (20.0 g, 95.5 mmol) and the reaction mixture was then heated to 80° C. for 16 hrs. Subsequently the reaction was cooled to 25° C. and the EtOH was removed under reduced pressure. Ethyl acetate (150 mL) was added followed by slow addition of 1 N HCl to adjust to pH=4. The organic layer was then separated, dried over Na₂SO₄ and concentrated to afford 3-oxo-2-phenyl-2,3-dihydro-1H-pyrazole-4-carboxylic acid ethyl ester (20.5 g, 95%) as an orange solid. ¹H-NMR (400 MHz, d-CDCl₃) δ 7.80-7.75 (m, 3H), 7.47-7.42 (m, 2H), 7.33-7.29 (m, 1H), 4.34 (q, 2H), 1.37 (t, 3H).

### Step B

### 2-Phenyl-2,4-dihydro-pyrazol-3-one

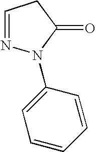

To a solution of 3-oxo-2-phenyl-2,3-dihydro-1H-pyrazole-4-carboxylic acid ethyl ester (20.5 g, 88.1 mmol) in MeOH (150 mL) was added 1 N NaOH (220 mL, 440 mmol) and the reaction mixture was heated to 70° C. for 7 hrs. The reaction mixture was then cooled to 0° C. and concentrated HCl was added to adjust to pH=2. The acidified reaction mixture was then heated to 100° C. for 14 hrs. Subsequently, the reaction mixture was cooled to 25° C. and ethyl acetate (400 mL) and water (200 mL) were added. The organic layer was separated, washed with brine, dried over Na₂SO₄ and concentrated to a brown oil that was purified by column chromatography (20-50% ethyl acetate/hexane) to provide 2-phenyl-2,4-dihydro-pyrazol-3-one (5.42 g, 38%). ¹H-NMR (400 MHz, d-CDCl₃) δ 7.84 (d, 2H), 7.46 (s, 1H), 7.41-7.35 (m, 2H), 7.21-7.17 (m, 1H), 3.47 (s, 2H).

### Step C

### 5-(2-Nitro-phenoxy)-1-phenyl-1H-pyrazole

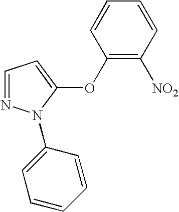

To a solution of 1-fluoro-2-nitrobenzene (4.78 g, 33.8 mmol) and 2-phenyl-2,4-dihydro-pyrazol-3-one (5.42 g, 33.8 mmol) in DMF (65 mL) was added potassium carbonate (5.14 g, 37.2 mmol), and the reaction mixture was stirred at 80° C. for 16 hrs. Subsequently, water (100 mL) and ethyl acetate (100 mL) were added. The organic layer was separated, washed with brine, dried over Na₂SO₄ and concentrated to a brown oil that was purified by column chromatography (15-25% ethyl acetate/hexane) to provide 5-(2-nitro-phenoxy)-1-phenyl-1H-pyrazole (2.50 g, 26%). ¹H-NMR (400 MHz, d-DMSO) δ 8.07 (d, 1H), 7.74-7.70 (m, 1H), 7.66-7.62 (m, 2H), 7.49-7.40 (m, 5H), 7.35-7.31 (m, 1H), 5.92 (s, 1H).

### Step D

### 2-(2-Phenyl-2H-pyrazol-3-yloxy)-phenylamine

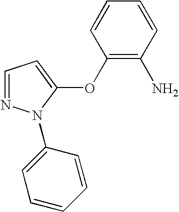

To a solution of 5-(2-nitro-phenoxy)-1-phenyl-1H-pyrazole (2.50 g, 8.89 mmol) in MeOH (50 mL) under a nitrogen atmosphere was added 10% Pd/C (200 mg). The reaction vessel was evacuated, flushed with nitrogen and then filled with hydrogen via balloon. The reaction mixture was stirred at 25° C. for 6 hrs after which time the reaction vessel was flushed with nitrogen and filtered through a pad of celite. The filtrate was concentrated to an oil that was purified by column chromatography (10% ethyl acetate/hexane) to provide 2-(2-phenyl-2H-pyrazol-3-yloxy)-phenylamine (2.05 92%). ¹H-NMR (400 MHz, d-DMSO) δ 7.76 (d, 2H), 7.51-7.44 (m, 3H), 7.33-7.29 (m, 1H), 6.90-6.89 (m, 2H), 6.74 (d, 1H), 6.49-6.45 (m, 1H), 5.56 (s, 1H).

### Step E

### 1-[2-(2-Phenyl-2H-pyrazol-3-yloxy)-phenyl]-3-(4-trifluoromethoxy-phenyl)-urea

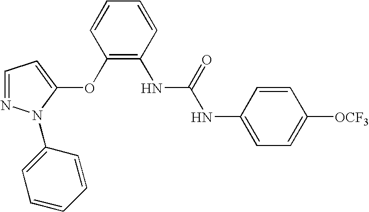

To a solution of 2-(2-phenyl-2H-pyrazol-3-yloxy)-phenylamine (0.58 g, 2.31 mmol) in THF (15 mL) was added 4-(trifluoromethoxy)-phenyl isocyanate (0.47 g, 2.31 mmol) and triethyl amine (0.35 g, 3.47 mmol). The reaction mixture was stirred at 65° C. for 6 hrs and then cooled to 25° C. The solvent was removed under reduced pressure and the resulting residue was purified by column chromatography (15-25% ethyl acetate/hexane) to afford 1-[2-(2-Phenyl-2H-pyrazol-3-yloxy)-phenyl]-3-(4-trifluoromethoxy-phenyl)-urea (0.32 g, 30%). ¹H-NMR (400 MHz, d-CHCl3) δ 8.61 (s, 1H), 8.28 (d, 1H), 7.75 (s, 1H), 7.50-7.46 (m, 3H), 7.33-7.16 (m, 6H), 7.05-6.97 (m, 5H).

### EXAMPLE 25

### 1-{2-[2-(2-Chloro-phenyl)-2H-pyrazol-3-yloxy]-phenyl}-3-(4-trifluoromethoxy-phenyl)-urea

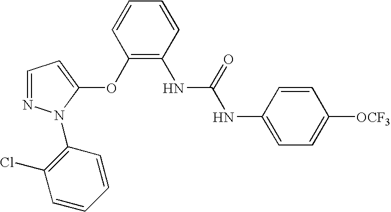

Prepared according to the method of Example 24. ¹H-NMR (400 MHz, d-DMSO) δ 9.28 (s, 1H), 824 (s, 1H), 8.02 (d, 1H), 7.62 (d, 1H), 7.60-7.40 (m, 6H), 7.25 (d, 2H), 7.09-6.93 (m, 3H), 5.90 (s, 1H).

### EXAMPLE 26

### 1-Phenyl-5-{2-[3-(4-trifluoromethoxy-phenyl)-ureido]-phenoxy}-1H-pyrazole-3-carboxylic acid methyl ester

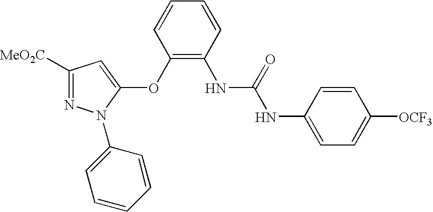

### Step A

### 5-Oxo-1-phenyl-4,5-dihydro-1H-pyrazole-3-carboxylic acid methyl ester

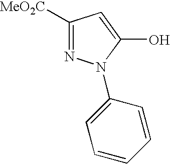

To a solution of phenyl hydrazine (8.0 g, 56.3 mmol) in MeOH (100 mL) at 25° C. was added dimethyl acetylene dicarboxylate (6.1 g, 56.3 mmol). Reaction mixture was stirred at 25° C. for 18 hrs as a yellow precipitate developed. The MeOH was then removed under reduced pressure and xylenes (100 mL) were added, and the reaction mixture was heated to 80° C. for 2 hrs. The reaction was slowly cooled to 25° C. and product precipitated as a white solid. Solid was isolated by filtration, washed with hexane and dried under high vacuum to afford 5-oxo-1-phenyl-4,5-dihydro-1H-pyrazole-3-carboxylic acid methyl ester (7.25 g, 59%) as a mixture of tautomers. MS (APCI) [M+H] 219.13.

### Step B

### 5-(2-Nitro-phenoxy)-1-phenyl-1H-pyrazole-3-carboxylic acid methyl ester

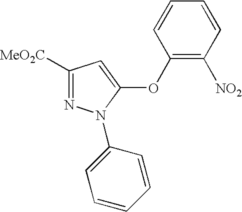

To a solution of 1-fluoro-2-nitrobenzene (1.94 g, 13.8 mmol) and 5-oxo-1-phenyl-4,5-dihydro-1H-pyrazole-3-carboxylic acid methyl ester (3.0 g, 13.7 mmol) in DMSO (30 mL) was added potassium carbonate (2.85 g, 20.6 mmol), and the reaction mixture was stirred at 875° C. for 16 hrs. Subsequently, water (100 mL) and ethyl acetate (100 mL) were added. The organic layer was separated, washed with brine, dried over Na₂SO₄ and concentrated to a brown oil that was purified by column chromatography (10-20% ethyl acetate/hexane) to provide 5-(2-nitro-phenoxy)-1-phenyl-1H-pyrazole-3-carboxylic acid methyl ester (0.48 g, 10%). ¹H-NMR (400 MHz, d-CHCl3) δ 8.02 (d, 1H), 7.77-7.74 (m, 2H), 7.62-7.57 (m, 1H), 7.46-7.43 (m, 2H), 7.37-7.24 (m, 3H), 6.29 (s, 1H), 3.92 (s, 3H).

### Step C

### 5-(2-Amino-phenoxy)-1-phenyl-1H-pyrazole-3-carboxylic acid methyl ester

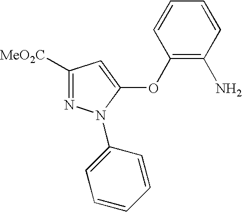

To a solution of 5-(2-nitro-phenoxy)-1-phenyl-1H-pyrazole-3-carboxylic acid methyl ester (0.48 g, 1.42 mmol) in MeOH (50 mL) under a nitrogen atmosphere was added 10% Pd/C (100 mg). The reaction vessel was evacuated, flushed with nitrogen and then filled with hydrogen via balloon. The reaction mixture was stirred at 25° C. for 14 hrs after which time the reaction vessel was flushed with nitrogen and filtered through a pad of celite. The filtrate was concentrated to provide 5-(2-amino-phenoxy)-1-phenyl-1H-pyrazole-3-carboxylic acid methyl ester (0.25 57%). ¹H-NMR (400 MHz, d-CDCl₃) δ 7.77 (d, 2H), 7.47-7.34 (m, 3H), 7.05-7.00 (m, 2H), 6.81-6.70 (m, 2H), 6.17 (s, 1H), 3.90 (s, 3H), 3.73 (s, 2H).

### Step D

### 5-{2-[3-(4-tert-butyl-phenyl)-ureido]-phenoxy}-1-phenyl-1H-pyrazole-3-carboxylic acid methyl ester

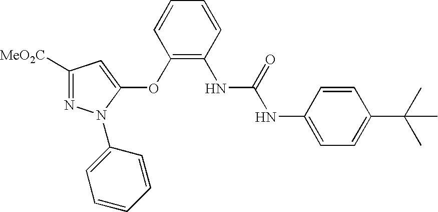

To a solution of 5-(2-amino-phenoxy)-1-phenyl-1H-pyrazole-3-carboxylic acid methyl ester (0.11 g, 0.36 mmol) in THF (15 mL) was added 4-(tert-butyl)-phenyl isocyanate (0.062 g, 0.36 mmol) and triethyl amine (0.072 g, 0.71 mmol). The reaction mixture was stirred at 65° C. for 14 hrs and then cooled to 25° C. The solvent was removed under reduced pressure and the resulting residue was purified by column chromatography (10-20% ethyl acetate/hexane) to afford 5-{2-[3-(4-tert-butyl-phenyl)-ureido]-phenoxy}-1-phenyl-1H-pyrazole-3-carboxylic acid methyl ester (0.085 g, 49%). ¹H-NMR (400 MHz, d-CHCl3) δ 8.98 (s, 1H), 8.42 (s, 1H), 8.15 (d, 1H), 7.77 (d, 2H), 7.52 (t, 2H), 7.24 (t, 1H), 7.25 (dd, 4H), 7.15-7.11 (m, 2H), 6.97-6.95 (m, 1H), 6.21 (s, 1H), 3.78 (s, 3H), 1.22 (s, 9H).

### EXAMPLE 27

### 5-{2-[3-(4-tert-Butyl-phenyl)-ureido]-phenoxy}-1-phenyl-1H-pyrazole-3-carboxylic acid

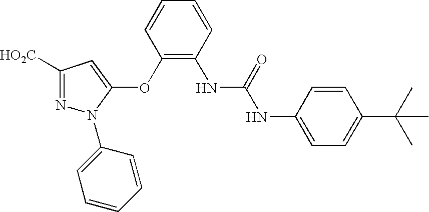

To a solution of 5-{2-[3-(4-tert-butyl-phenyl)-ureido]-phenoxy}-1-phenyl-1H-pyrazole-3-carboxylic acid methyl ester (0.065 g, 0.13 mmol) in MeOH:THF (1:1, 4 mL) was added 1 N NaOH (0.26 mL, 0.26 mmol). The reaction mixture was heated to 60° C. for 2 hrs after which time the reaction mixture was cooled to 25° C. and the solvent was removed under reduced pressure. Ethyl acetate (20 mL) and 1 N HCl (5 mL) were added, and the organic layer was separated, dried over Na₂SO₄ and concentrated to a yellow-brown oil. Addition of CH₂Cl₂ (5 mL) resulted in precipitation of a light brown solid that was isolated by filtration and dried under high vacuum to afford 5-{2-[3-(4-tert-Butyl-phenyl)-ureido]-phenoxy}-1-phenyl-1H-pyrazole-3-carboxylic acid (0.035 g, 55%). ¹H-NMR (400 MHz, d-CHCl3) δ 8.99 (s, 1H), 8.41 (s, 1H), 8.17 (d, 1H), 7.77 (d, 2H), 7.52 (t, 2H), 7.41-7.24 (m, 5H), 7.16-7.09 (m, 2H), 6.97-6.93 (m, 1H), 6.15 (s, 1H), 1.22 (s, 9H).

### EXAMPLE 28

### 1-[2-(5-Ethyl-2-phenyl-2H-pyrazol-3-yloxy)-phenyl]-3-(4-trifluoromethoxy-phenyl)-urea

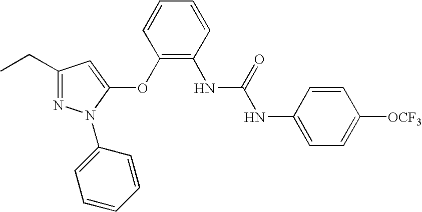

Prepared according to the method of Example 1 substituting 3-oxo-pentanoic acid methyl ester for ethyl acetoacetate in Step A. ¹H-NMR (400 MHz, d-DMSO) δ 9.33 (s, 1H), 8.51 (s, 1H), 8.16 (d, 1H), 7.70 (d, 2H), 7.51 (d, 2H), 7.42 (t, 2H), 7.28-7.24 (m, 3H), 7.14-6.94 (m, 3H), 5.68 (s, 1H), 2.52 (q, 2H), 1.12 (t, 3H).

### EXAMPLE 29

### 1-[2-(5-Isopropyl-2-phenyl-2H-pyrazol-3-yloxy)-phenyl]-3-(4-trifluoromethoxy-phenyl)-urea

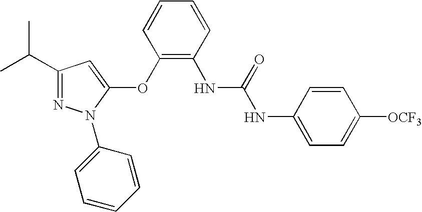

Prepared according to the method of Example 1 substituting 4-methyl-3-oxo-pentanoic acid methyl ester for ethyl acetoacetate in Step A. H-NMR (400 MHz, d-DMSO) δ 9.31 (s, 1H), 8.51 (s, 1H), 8.16 (d, 1H), 7.70 (d, 2H), 7.49 (d, 2H), 7.41 (t, 2H), 7.29-7.24 (m, 3H), 7.15-6.94 (m, 3H), 5.69 (s, 1H), 2.82 (sept, 1H), 1.16 (d, 6H).

### EXAMPLES 30-33

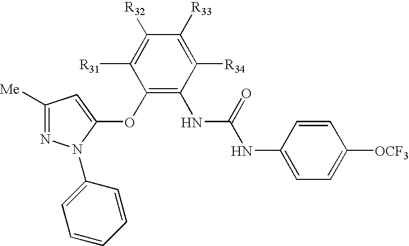

Examples 30-33 were prepared according to the methods of Example 1 and Example 2 using the appropriate commercially available substituted 2-halo-1-nitrobenzene analogs of structure (4) starting materials.

| Example | R₃₁ | R₃₂ | R₃₃ | R₃₄ | H-NMR |
|---|---|---|---|---|---|
| 30 | H | Me | F | H | ¹H-NMR (400 MHz, d-CDCl3) δ 8.12 (s, 1 |
| | | | | | H), 8.10 (dd, 1 H), 7.73 (s, 1 H), 7.50-7.48 |
| | | | | | (m, 2 H), 7.35-7.25 (m, 4 H), 7.22-7.20 (m, |
| | | | | | 1 H), 7.07 (s, 1 H), 7.05 (s, 1 H), 6.89 (d, 1 |
| | | | | | H), 5.26 (s, 1 H), 2.18 (d, 3 H), 2,16 (s, 3 |
| | | | | | H). |
| 31 | F | H | H | H | ¹H-NMR (400 MHz, d-CDCl3) δ 9.01 (s, 1 |
| | | | | | H), 8.42-8.38 (m, 1 H), 8.20 (s, 1 H), 7.69- |
| | | | | | 7.65 (m, 2 H), 7.35-7.22 (m, 6 H), 7.07- |
| | | | | | 7.00 (m, 2 H), 6.85-6.81 (m, 1 H), 5.27 (s, |
| | | | | | 1 H), 2.12 (s, 3 H). |
| 32 | H | H | CN | H | ¹H-NMR (400 MHz, d-DMSO) δ 9.48 (s, 1 |
| | | | | | H), 8.30 (br s, 1 H) 7.81 (s, 1 H), 7.80 (s, 1 |
| | | | | | H), 7.53-7.50 (m, 2 H), 7.46-7.38 (m, 5 H), |
| | | | | | 7.24-7.19 (m, 3 H), 2.12 (s, 3 H). |
| 33 | H | H | H | OMe | ¹H-NMR (400 MHz, d-DMSO) δ 8.93 (s, 1 |
| | | | | | H), 7.67-7.64(m, 3 H), 7.65(s, 1 H), 7.46 |
| | | | | | (s, 1 H), 7.31-7.21 (m, 2 H), 7.20-7.14 (m, |
| | | | | | 4 H), 6.92 (d, 1 H), 6.79 (d, 1 H) 5.47 (s, 1 |
| | | | | | H), 3.79 (s, 3 H), 2.10 (s, 3 H). |

### EXAMPLES 34-82

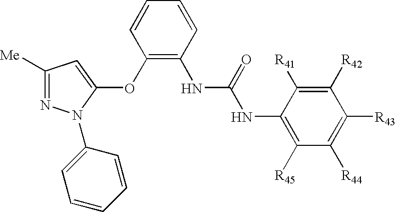

Examples 34-82 were prepared according to the following general procedure: A 0.2 M stock solution of 2-(5-methyl-2-phenyl-2H-pyrazol-3-yloxy)-phenylamine in anhydrous DMF was prepared. Aliquots (450 μL, 90 umol) of this solution were then added to individual vials of an appropriate R₁-R₅-substituted phenyl isocyanate (0.2 M in DMF). The vials were sealed and placed in a heated shaker at 75° C. for 16 h. Remove the volatile solvents in vacuo using the GeneVac™ or SpeedVac™ at 45° C. for 8 h. The samples were then purified by preparative reverse phase high pressure liquid chromatography. Method; Waters Alliance 2795 LC system, Column: Gemini C₁₈, 100×21.2 mm, 5 micron; 214 nM detection; mobile phase (A) acetonitrile, (B) acetonitrile/water (80/20) T=0 minutes (70% B), T=1 minutes (70% B), T=10 minutes (2% B), T=10.5 minutes (2% B), T=10.6 minutes (70% B); flow rate 25.5 ml/minute. Examples 34-83 were subsequently analyzed by LC/MS (unless otherwise indicated) with data provided in the table below.

| Example | R₄₁ | R₄₂ | R₄₃ | R₄₄ | R₄₅ | LC/MS Data* |
|---|---|---|---|---|---|---|
| 34 | F | H | H | F | H | m/z 420, Tr = 4.56 min. |
| 35 | F | H | F | H | H | m/z 420, Tr = 4.43 min. |
| 36 | H | CF₃ | H | H | H | ¹H-NMR (400 MHz, d-DMSO) δ 8.80 (s, 1 |
| | | | | | | H), 8.32 (s, 1 H), 7.81 (s, 1 H), 7.60 (s, 1 |
| | | | | | | H), 7.50 (d, 3 H), 7.31-7.05 (m, 6 H), 5.30 |
| | | | | | | (s, 1 H), 2.21 (s, 3 H). |
| 37 | H | F | H | F | H | m/z 420, Tr = 4.55 min. |
| 38 | H | F | H | H | H | m/z 402, Tr = 4.39 min. |
| 39 | H | H | SMe | H | H | m/z 430, Tr = 8.46 min |
| 40 | H | H | F | H | H | m/z 402, Tr = 4.31 min. |
| 41 | H | H | OEt | H | H | m/z 428.18, Tr = 8.06 min. |
| 42 | H | Cl | OMe | H | H | m/z 448.13, Tr = 8.31 min. |
| 43 | H | Me | H | Me | H | m/z 412.19, Tr = 7.86 min. |
| 44 | H | SMe | H | H | H | m/z 412.19, Tr = 7.88 min |
| 45 | F | F | F | H | H | m/z 438, Tr = 4.54 min. |
| 46 | H | CN | H | H | H | m/z 409.15, Tr = 7.74 min. |
| 47 | Cl | H | H | H | H | m/z 418.12, Tr = 8.51 min. |
| 48 | Me | H | H | F | H | m/z 416.16, Tr = 8.34 min. |
| 49 | CF₃ | H | F | H | H | m/z 440.22, Tr = 8.35 min. |
| 50 | Et | H | H | H | Me | m/z 470.14, Tr = 7.75 min. |
| 51 | H | CO₂Me | H | H | H | m/z 432.14, Tr = 8.76 min. |
| 52 | CF₃ | H | H | H | F | m/z 428.18, Tr = 8.63 min. |
| 53 | CF₃ | H | H | H | H | m/z 403.16, Tr = 6.12 min. |
| 54 | Br | H | F | H | F | m/z 414.17, Tr = 7.8 min. |
| 55 | Me | H | H | Cl | H | m/z 456.18, Tr = 7.76 min. |
| 56 | OMe | H | OMe | Cl | H | m/z 432.14, Tr = 8.79 min. |
| 57 | H | OMe | OMe | OMe | H | m/z 474, Tr = 4.06 min. |
| 58 | Me | H | H | H | Me | m/z 412, Tr = 4.23 min. |
| 59 | Cl | H | H | H | CF₃ | m/z 486, Tr = 4.26 min. |
| 60 | Me | H | Me | H | H | m/z 412, Tr = 4.43 min. |
| 61 | Cl | H | H | H | Cl | m/z 452, Tr = 4.22 min. |
| 62 | Cl | H | H | H | Me | m/z 432, Tr = 4.25 min. |
| 63 | H | C(O)Me | H | H | H | m/z 426, Tr = 4.13 min. |
| 64 | OMe | H | H | OMe | H | m/z 444, Tr = 4.37 min. |
| 65 | tBu | H | H | H | H | m/z 478.14, Tr = 8.54 min. |
| 66 | Me | H | OMe | H | H | m/z 412.19, Tr = 7.76 min. |
| 67 | H | OMe | H | H | H | m/z 428.18, Tr = 7.4 min. |
| 68 | OMe | H | H | Cl | H | m/z 412.19, Tr = 7.98 min. |
| 69 | SMe | H | H | H | H | m/z 448.13, Tr = 9.02 min. |
| 70 | F | H | H | H | F | m/z 430.15, Tr = 8.18 min. |
| 71 | OMe | H | H | Me | H | m/z 442.16, Tr = 7.93 min. |
| 72 | Et | H | H | H | Et | m/z 414.17, Tr = 7.61 min. |
| 73 | H | H | OMe | H | H | m/z 416.16, Tr = 8.34 min. |
| 74 | OMe | H | H | H | H | m/z 442.24, Tr = 9.52 min. |
| 75 | Me | H | Br | H | Me | m/z 490.1, Tr = 8.43 min. |
| 76 | tBu | H | Me | H | Me | m/z 426.21, Tr = 7.96 min. |
| 77 | Me | H | H | H | tBu | m/z 454.24, Tr = 8.46 min. |
| 78 | OMe | H | OMe | H | H | m/z 444.18, Tr = 7.88 min. |
| 79 | Me | H | H | Me | H | m/z 412.19, Tr = 8.28 min. |
| 80 | H | OMe | H | OMe | H | m/z 444.18, Tr = 7.93 min. |
| 81 | OEt | H | H | H | H | m/z 442.16, Tr = 7.58 min. |
| 82 | Br | H | H | H | H | m/z 462.07, Tr = 8.57 min. |

*LC/MS data provided unless otherwise indicated.

### EXAMPLE 83

### 2-[4-(3-{2-[2-(2-Chloro-phenyl)-5-methyl-2H-pyrazol-3-yloxy]-phenyl}-ureido)-phenyl]-2-methyl-propionic acid ethyl ester

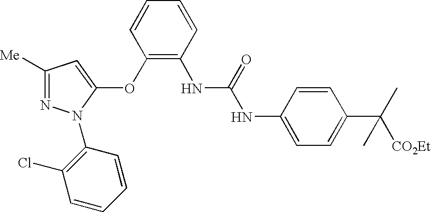

### Step A

### 2-Methyl-2-(4-nitro-phenyl)-propionic acid ethyl ester

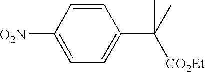

To a solution of NaOtBu (2.89 g, 30.1 mmol) in DMF (100 mL) at 0° C. was added (4-nitro-phenyl)-acetic acid ethyl ester (6.00 g, 28.7 mmol) resulting in a deep purples solution. The reaction mixture was stirred at 0° C. for 5 min and then methyl iodide (4.28 g, 30.1 mmol) was slowly added. The resulting reaction mixture was then stirred at 0° C. for 0.5 hr. Subsequently, a second portion of NaOtBu (2.89 g, 30.1 mmol) was added and after stirring for 5 min, a second portion of methyl iodide (4.28 g, 30.1 mmol) was slowly added. The reaction mixture was stirred at 0° C. for 0.5 hr and then quenched with dilute acetic acid (150 mL) and extracted with ethyl acetate (200 mL). The organic layer was dried over Na₂SO₄, concentrated and purified by column chromatography (2-5% ethyl acetate/hexane) to provide 2-methyl-2-(4-nitro-phenyl)-propionic acid ethyl ester (3.41 g, 50%). ¹H-NMR (400 MHz, d-CHCl3) δ 8.16 (d, 2H), 7.48 (d, 2H), 4.11 (q, 2H), 1.59 (s, 6H), 1.16 (t, 3H).

### Step B

### 2-(4-Amino-phenyl)-2-methyl-propionic acid ethyl ester

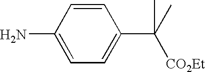

To a solution of 2-methyl-2-(4-nitro-phenyl)-propionic acid ethyl ester (3.40 g, 14.3 mmol) in EtOH (100 mL) under a nitrogen atmosphere was added 10% Pd/C (300 mg). The reaction vessel was evacuated, flushed with nitrogen and then filled with hydrogen via balloon. The reaction mixture was stirred at 25° C. for 5 hrs after which time the reaction vessel was flushed with nitrogen and filtered through a pad of celite. The filtrate was concentrated and purified by column chromatography (20% ethyl acetate/hexane) to provide 2-(4-amino-phenyl)-2-methyl-propionic acid ethyl ester (2.35 579%). ¹H-NMR (400 MHz, d-CDCl₃) δ 7.14 (d, 2H), 6.63 (d, 2H), 4.08 (q, 2H), 3.68 (bs, 2H), 1.51 (s, 6H), 1.16 (t, 3H).

### Step C

### 1-(2-Chloro-phenyl)-5-(2-isocyanato-phenoxy)-3-methyl-1H-pyrazole

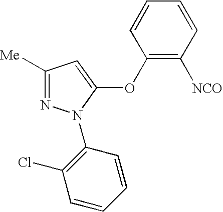

To a solution of 2-[2-(2-chloro-phenyl)-5-methyl-2H-pyrazol-3-yloxy]-phenylamine [from Example 2, Step B] (0.982 g, 3.73 mmol) in CH₂Cl₂ (50 mL) at 0° C. was added disphosgene (0.51 g, 2.73 mmol) followed by a solution of proton sponge (1.40 g, 6.60 mmol) in CH₂Cl₂ (20 mL). The reaction mixture was stirred for 1 hr while warming to 25° C. The reaction mixture was then washed with 1 N HCl, 1 N NaOH and brine prior to drying over Na₂SO₄. The organic layer was concentrated to provide 1-(2-chloro-phenyl)-5-(2-isocyanato-phenoxy)-3-methyl-1H-pyrazole (0.95 g, 96%). ¹H-NMR (400 MHz, d-CDCl3) δ 7.48 (d, 2H), 7.37-7.31 (m, 2H), 7.18 (d, 1H), 7.14-6.99 (m, 3H), 5.61 (s, 1H), 2.30 (s, 3H).

### Step D

### 2-[4-(3-{2-[2-(2-Chloro-phenyl)-5-methyl-2H-pyrazol-3-yloxy]-phenyl}-ureido)-phenyl]-2-methyl-propionic acid ethyl ester

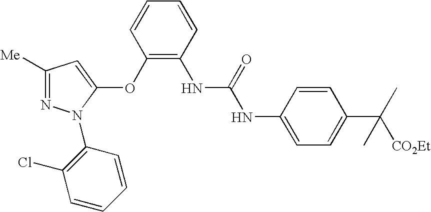

To a solution of 2-(4-amino-phenyl)-2-methyl-propionic acid ethyl ester (0.60 g, 2.92 mmol) in THF (20 mL) was added 1-(2-chloro-phenyl)-5-(2-isocyanato-phenoxy)-3-methyl-1H-pyrazole (0.95 g, 2.92 mmol) and triethyl amine (0.44 g, 4.37 mmol). The reaction mixture was stirred at 60° C. for 17 hrs and then cooled to 25° C. The solvent was removed under reduced pressure and the resulting residue was purified by column chromatography (25-35% ethyl acetate/hexane) to afford 2-[4-(3-{2-[2-(2-chloro-phenyl)-5-methyl-2H-pyrazol-3-yloxy]-phenyl}-ureido)-phenyl]-2-methyl-propionic acid ethyl ester (1.05 g, 68%). ¹H-NMR (400 MHz, d-DMSO) δ 9.11 (s, 1H), 8.17 (s, 1H), 8.03 (d, 1H), 7.58-7.54 (m, 2H), 7.45-7.32 (m, 2H), 7.18 (d, 2H), 7.08-7.04 (m, 2H), 6.95-6.91 (m, 1H), 5.67 (s, 1H), 4.02 (q, 2H), 1.43 (s, 6H), 1.07 (t, 3H).

### EXAMPLE 84

### 2-[4-(3-{2-[2-(2-Chloro-phenyl)-5-methyl-2H-pyrazol-3-yloxy]-phenyl}-ureido)-phenyl]-2-methyl-propionic acid

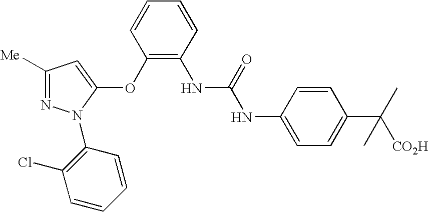

To a solution of 2-[4-(3-{2-[2-(2-chloro-phenyl)-5-methyl-2H-pyrazol-3-yloxy]-phenyl}-ureido)-phenyl]-2-methyl-propionic acid ethyl ester (0.198 g, 0.37 mmol) in MeOH:THF (1:1, 10 mL) was added 1 N LiOH (3.71 mL, 3.71 mmol). The reaction mixture was heated to reflux for 75° C. for 2 hrs after which it was cooled to 25° C. and the solvent was removed under reduced pressure. Ethyl acetate (50 mL) and 1 N HCl (20 mL) were then added and the organic layer was separated dried over Na₂SO₄ and concentrated to a semi-solid that was purified by column chromatography (50% ethyl aceate/hexane) to provide 2-[4-(3-{2-[2-(2-Chloro-phenyl)-5-methyl-2H-pyrazol-3-yloxy]-phenyl}-ureido)-phenyl]-2-methyl-propionic acid (0.057 g, 40%). ¹H-NMR (400 MHz, d-CHCl3) δ 9.10 (s, 1H), 8.17 (s, 1H), 8.03 (d, 1H), 7.58-7.54 (m, 2H), 7.45-7.40 (m, 2H), 7.33 (d, 2H), 7.21 (d, 2H), 7.05 (d, 2H), 6.94-6.90 (m, 1H), 5.67 (s, 1H), 1.41 (s, 6).

### EXAMPLE 85

### 1-{2-[2-(2-Chloro-phenyl)-5-methyl-2H-pyrazol-3-yloxy]-phenyl}-3-[4-(2-hydroxy-1,1-dimethyl-ethyl)-phenyl]-urea

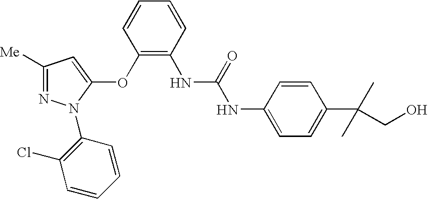

To a solution of 2-[4-(3-{2-[2-(2-chloro-phenyl)-5-methyl-2H-pyrazol-3-yloxy]-phenyl}-ureido)-phenyl]-2-methyl-propionic acid ethyl ester (0.465 g, 0.872 mmol) in THF (50 mL) at 0° C. was slowly added a solution of lithium aluminum hydride (1.74 mL of 1.0 M in 1.0 M ethe, 1.74 mmol). The reaction mixture was stirred at 0° C. for 0.5 hr and then at 25° C. for 3 hrs. The reaction mixture was quenched by slow addition of saturated ammonium chloride solution (10 mL) followed by the addition of water (50 mL), ethyl acetate (100 mL) and 1 N HCl (50 mL). The organic layer was separated, dried over Na₂SO₄ and concentrated to an oil that was purified by column chromatography (30-65% ethyl acetate/hexane) to provide 1-{2-[2-(2-chloro-phenyl)-5-methyl-2H-pyrazol-3-yloxy]-phenyl}-3-[4-(2-hydroxy-1,1-dimethyl-ethyl)-phenyl]-urea (0.35 g, 81%). ¹H-NMR (400 MHz, d-CHCl3) δ 9.02 (s, 1H), 8.14 (s, 1H), 8.06 (d, 1H), 7.59-7.55 (m, 2H), 7.46-7.39 (m, 2H), 7.96-7.39 (m, 2H), 7.25 (d, 2H), 7.21 (d, 2H), 7.08-7.04 (m, 2H), 6.99-6.90 (m, 1H), 5.67 (s, 1H), 4.58 (t, 1H), 3.34 (d, 2H), 2.15 (s, 3H), 1.16 (s, 6H).

### EXAMPLE 86

### 4-(3-{2-[2-(2-Chloro-phenyl)-5-methyl-2H-pyrazol-3-yloxy]-phenyl}-ureido)-benzoic acid methyl ester

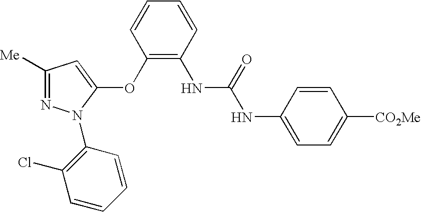

2-[2-(2-Chloro-phenyl)-5-methyl-2H-pyrazol-3-yloxy]-phenylamine [Example 2, Step B] (3.0 g, 10.0 mmol) and 4-isocyanato-benzoic acid methyl ester (1.95 g, 11 mmol) were added to tetrahydrofuran (50 mL). The solution was heated at 65° C. for 20 hrs. The reaction was cooled and concentrated under vacuum to a gum. The residue was purified by medium pressure liquid chromatography to give 4-(3-{2-[2-(2-chloro-phenyl)-5-methyl-2H-pyrazol-3-yloxy]-phenyl}-ureido)-benzoic acid methyl ester (4.8 g, 100%) as a white solid. ¹H-NMR (400 MHz, d-DMSO) δ 9.47 (s, 1H), 8.29 (s, 1H), 8.01 (d, 1H), 7.83 (m, 2H), 7.56-7.36 (m, 6H), 7.09-6.93 (m, 3H), 5.68 (s, 1H), 3.76 (s, 3H), 2.14 (s, 3H).

### EXAMPLE 87

### 1-{2-[2-(2-Chloro-phenyl)-5-methyl-2H-pyrazol-3-yloxy]-phenyl}-3-(4-hydroxymethyl-phenyl)-urea

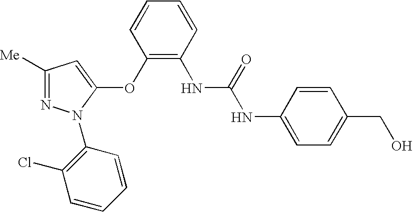

4-(3-{2-[2-(2-Chloro-phenyl)-5-methyl-2H-pyrazol-3-yloxy]-phenyl}-ureido)-benzoic acid methyl ester [Example 86] (4.83 g, 10.13 mmol) was dissolved in tetrahydrofuran (50 mL). 1M LiBH₄ in diethylether (15.2 mmol, 7.6 ml) was added and the reaction was heated to 60 C for 22 h. 10 ml water was added and stirred for ½ hour. The reaction was extracted 2×50 ml ethyl acetate, dried over anhydrous sodium sulfate, filtered, and concentrated. Column chromatograohy gave 1-{2-[2-(2-chloro-phenyl)-5-methyl-2H-pyrazol-3-yloxy]-phenyl}-3-(4-hydroxymethyl-phenyl)-urea (1.0 g, 22%) as a colorless glassy solid. ¹H-NMR (400 MHz, d-DMSO) δ 9.05 (s, 1H), 8.13 (s, 1H), 8.03 (d, 1H), 7.57-7.53 (m, 2H), 7.44-7.39 (m, 2H), 7.30 (m, 2H), 7.17 (m, 2H), 7.03 (m, 2H), 6.93-6.89 (m, 1H), 5.85 (s, 1H), 5.00 (t, 1H), 4.37 (m, 2H), 2.13 (s, 3H).

### EXAMPLE 88

### 1-{2-[2-(3-Chloro-2-fluoro-phenyl)-5-methyl-2H-pyrazol-3-yloxy]-phenyl}-3-(4-trifluoromethoxy-phenyl)-urea

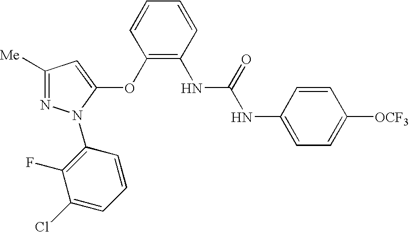

Prepared according to the method of Example 1 using 3-chloro-2-fluorophenyl hydrazine hydrochloride in place of o-tolyl-hydrazine hydrochloride. ¹H-NMR (400 MHz, d-CDCl₃) δ 8.23 (dd, 1H), 7.50-7.34 (m, 4H), 7.24-7.10 (m, 5H), 7.05-7.00 (m, 3H), 5.46 (s, 1H), 2.23 (s, 3H).

### EXAMPLE 89

### 1-{2-[2-(2-Chloro-phenyl)-5-methyl-2H-pyrazol-3-yloxy]-phenyl}-3-(4-cyclohexyl-phenyl)-urea

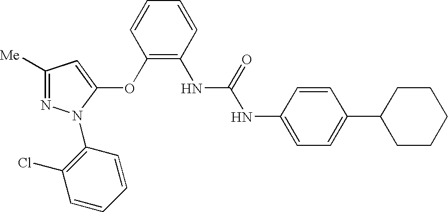

Prepared according to the method of Example 83 [Step D] using 4-cyclohexylaniline in place of 2-(4-amino-phenyl)-2-methyl-propionic acid ethyl ester. ¹H-NMR (400 MHz, d-DMSO) δ 9.01 (s, 1H), 8.13 (s, 1H), 8.05 (dd, 1H), 7.59-7.54 (m, 2H), 7.46-7.39 (m, 2H), 7.29 (s, 1H), 7.27 (s, 1H), 7.10-7.04 (m, 4H), 6.95-6.90 (m, 1H), 5.70 (s, 1H), 2.41-2.39 (m, 1H), 2.15 (s, 3H), 1.75-1.60 (m, 5H), 1.38-1.19 (m, 5H).

### EXAMPLE 90

### 1-{2-[2-(2-Ethyl-phenyl)-5-methyl-2H-pyrazol-3-yloxy]-pyridin-3-yl}-3-(4-trifluoromethoxy-phenyl)-urea

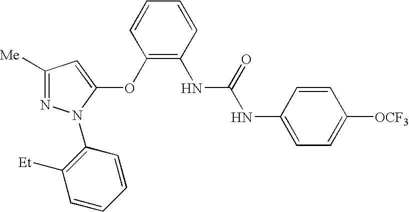

Prepared according to the method of Example 1 using 2-ethylphenyl hydrazine hydrochloride in place of o-tolyl-hydrazine hydrochloride in Step A and 2-chloro-3-nitropyridine in place of 1-fluoro-2-nitrobenzene in Step B. ¹H-NMR (400 MHz, d-DMSO) δ 9.38 (s, 1H), 8.44 (s, 1H), 8.34 (d, 1H), 7.65 (d, 1H), 7.49 (d, 2H), 7.27-7.02 (m, 7 J), 6.00 (s, 1H), 2.49-2.45 (m, 2H), 2.21 (s, 3H), 1.00 (t, 3H).

### EXAMPLE 91

### 1-(4-(phenyl)phenyl)-3-(2-(1-(2-ethylphenyl)-3-methyl-1H-pyrazol-5-yloxy)phenyl)urea

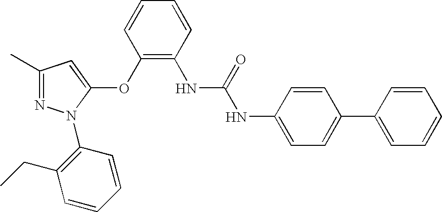

### Step A

### 1-(2-ethylphenyl)-3-methyl-1H-pyrazol-5-ol

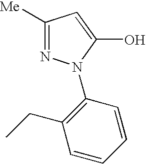

Prepared according to the method of Example 1 (Step A) substituting 2-ethyl phenyl hydrazine hydrochloride for o-tolyl-hydrazine hydrochloride. ¹H-NMR (400 MHz, d-DMSO) δ 10.91 (s, 1H), 7.36-7.08 (m, 4H), 5.27 (s, 1H), 2.08 (s, 3H), 2.51-2.38 (m, 3H), 2.05 (s, 3H), 1.05-0.95 (m, 3H).

### Step B

### 1-(2-ethylphenyl)-3-methyl-5-(2-nitrophenoxy)-1H-pyrazole

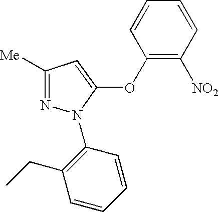

Prepared according to the method of Example 1 (Step B) substituting 1-(2-ethylphenyl)-3-methyl-1H-pyrazol-5-ol for 3-methyl-2-o-tolyl-2H-pyrazol-3-ol. ¹H-NMR (400 MHz, d-CDCl₃) δ 7.90 (dd, 1H), 7.62-7.58 (m, 2H), 7.35-7.19 (m, 5H), 5.60 (s, 1H), 2.58-2.55 (q, 2H), 2.35 (s, 3H), 1.09 (t, 3H).

### Step C

### 2-(1-(2-ethylphenyl)-3-methyl-1H-pyrazol-5-yloxy)benzenamine

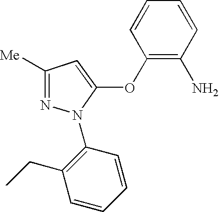

Prepared according to the method of Example 1 (Step C) substituting 1-(2-ethylphenyl)-3-methyl-5-(2-nitrophenoxy)-1H-pyrazole for 3-methyl-5-(2-nitro-phenoxy)-1-o-tolyl-1H-pyrazole. MS (APCI) [M+H] 294.2.

### Step D

### 1-(2-ethylphenyl)-5-(2-isocyanatophenoxy)-3-methyl-1H-pyrazole

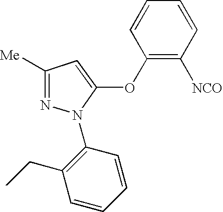

Prepared according to the method of Example 83 (Step C) substituting 2-(1-(2-ethylphenyl)-3-methyl-1H-pyrazol-5-yloxy)benzenamine for 2-(1-(2-chlorophenyl)-3-methyl-1H-pyrazol-5-yloxy)benzenamine. MS (APCI) [M+H] 320.1.

### Step E

### 1-(4-(phenyl)phenyl)-3-(2-(1-(2-ethylphenyl)-3-methyl-1H-pyrazol-5-yloxy)phenyl)urea

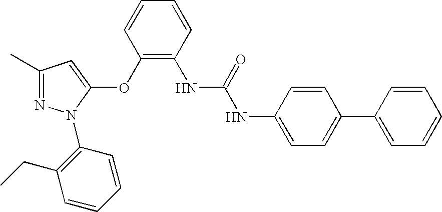

Prepared according to the method of Example 83 (Step D) substituting 4-phenylaniline in place of 2-(4-amino-phenyl)-2-methyl-propionic acid ethyl ester. ¹H NMR (400 MHz, d-DMSO) δ 9.23 (s, 1H) 8.22 (s, 1H) 8.04 (dd, 1H) 7.55-7.64 (m, 4H) 7.47-7.53 (m, 2H) 7.36-7.44 (m, 3H) 7.31-7.35 (m, 2H) 7.26-7.31 (m, 1H) 7.19-7.25 (m, 1H) 7.03-7.10 (m, 1H) 6.91-7.00 (m, 2H) 5.71 (s, 1H) 2.47-2.55 (m, 2H) 2.16 (s, 3H) 1.04 (t, 3H)

### EXAMPLE 92

### 1-(2-(1-(2-ethylphenyl)-3-methyl-1H-pyrazol-5-yloxy)phenyl)-3-(4-phenoxyphenyl)urea

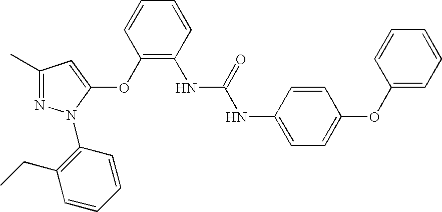

Prepared according to the method of Example 91 substituting 4-phenoxyaniline in place of 4-phenylaniline in Step D of this procedure. ¹H NMR (400 MHz, d-DMSO-d₆) δ 9.13 (s, 1H) 8.15 (s, 1H) 8.02 (dd, 1H) 7.39-7.44 (m, 2H) 7.35-7.38 (m, 1H) 7.30-7.35 (m, 4H) 7.19-7.26 (m, 1H) 7.02-7.09 (m, 2H) 6.89-6.98 (m, 6H) 5.69 (s, 1H) 2.47-2.55 (m, 2H) 2.16 (s, 3H) 1.04 (t, 3H)

### EXAMPLE 93

### 1-(2-(1-(2-ethylphenyl)-3-methyl-1H-pyrazol-5-yloxy)phenyl)-3-(2-fluoro-4-(trifluoromethyl)phenyl)urea

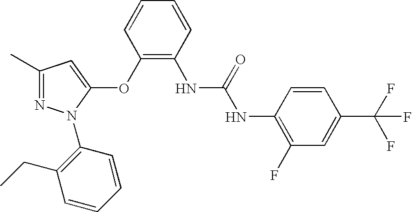

Prepared according to the method of Example 91 substituting 2-fluoro-4-(trifluoromethyl)benzenamine in place of 4-phenylaniline in Step D of this procedure. ¹H NMR (400 MHz, d-DMSO) δ 9.37 (d, 1H) 8.85 (s, 1H) 8.38 (t, 1H) 7.99 (d, 1H) 7.67 (dd, 1H) 7.51 (d, 1H) 7.34 (d, 1H) 7.29-7.33 (m, 2H) 7.16-7.24 (m, 1H) 7.04-7.10 (m, 1H) 6.96-7.01 (m, 2H) 5.73 (s, 1H) 2.46-2.53 (m, 2H) 2.16 (s, 3H) 1.02 (t, 3H)

### EXAMPLE 94

### 1-(2-chloro-4-(trifluoromethoxy)phenyl)-3-(2-(1-(2-ethylphenyl)-3-methyl-1H-pyrazol-5-yloxy)phenyl)urea

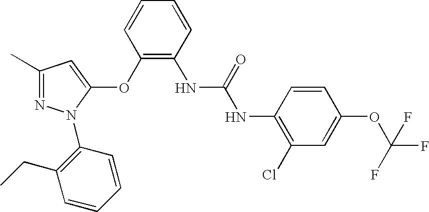

Prepared according to the method of Example 91 substituting 2-chloro-4-(trifluoromethoxy)benzenamine in place of 4-phenylaniline in Step D of this procedure. ¹H NMR (400 MHz, d-DMSO) δ 8.98 (s, 1H) 8.84 (s, 1H) 8.14 (d, 1H) 7.91 (dd, 1H) 7.57 (dd, 1H) 7.29-7.36 (m, 4H) 7.15-7.23 (m, 1H) 7.03-7.09 (m, 1H) 6.96-7.01 (m, 2H) 5.74 (d, 1H) 2.46-2.51 (m, 2H) 2.16 (s, 3H) 1.02 (t, 3H)

### EXAMPLE 95

### 1-(2-(1-(2-ethylphenyl)-3-methyl-1H-pyrazol-5-yloxy)phenyl)-3-phenylurea

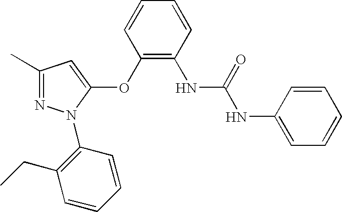

Prepared according to the method of Example 91 substituting aniline in place of 4-phenylaniline in Step D of this procedure. ¹H NMR (400 MHz, d-DMSO) δ 9.11 (s, 1H) 8.17 (s, 1H) 8.02 (dd, 1H) 7.31-7.43 (m, 5H) 7.18-7.29 (m, 3H) 7.02-7.09 (m, 1H) 6.89-6.99 (m, 3H) 5.69 (s, 1H) 2.47-2.55 (m, 2H) 2.16 (s, 3H) 1.03 (t, 3H)

### EXAMPLE 96

### 1-(4-(phenyl)-phenyl)-3-(2-(1-(2-chlorophenyl)-3-methyl-1H-pyrazol-5-yloxy)phenyl)urea

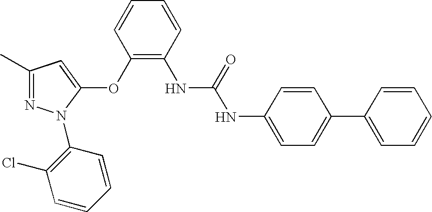

Prepared according to the method of Example 83 [Step D] using 4-phenyl aniline in place of 2-(4-amino-phenyl)-2-methyl-propionic acid ethyl ester. ¹H NMR (400 MHz, d-DMSO) δ 9.22 (s, 1H) 8.22 (s, 1H) 8.02-8.10 (m, 1H) 7.54-7.63 (m, 6H) 7.47-7.52 (m, 2H) 7.41-7.45 (m, 2H) 7.37-7.41 (m, 2H) 7.25-7.31 (m, 1H) 7.05-7.12 (m, 2H) 6.91-6.99 (m, 1H) 5.69 (s, 1H) 2.16 (s, 3H)

### EXAMPLE 97

### 1-(2-(1-(4-tert-butylphenyl)-3-methyl-1H-pyrazol-5-yloxy)phenyl)-3-(4-(trifluoromethoxy)phenyl)urea

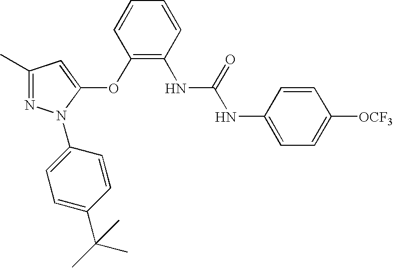

Prepared according to the method of Example 1 using 4-t-butylphenyl hydrazine hydrochloride in place of o-tolyl-hydrazine hydrochloride in Step A and 2-chloro-3-nitropyridine in place of 1-fluoro-2-nitrobenzene in Step B. ¹H NMR (400 MHz, d-CDCl₃) δ 8.44 (br. s., 1H) 8.32 (dd, 1H) 7.58 (s, 1H) 7.43-7.48 (m, 1H) 7.29-7.39 (m, 4H) 7.17-7.22 (m, 1H) 7.05-7.10 (m, 2H) 6.96-7.04 (m, 2H) 5.32 (s, 1H) 2.17 (s, 3H) 1.25 (s, 9H)

### EXAMPLE 98

### 1-(2-(1-(2,5-dichlorophenyl)-3-methyl-1H-pyrazol-5-yloxy)phenyl)-3-(4-(trifluoromethoxy)phenyl)urea

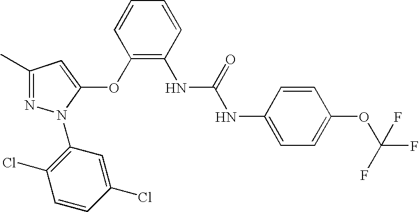

Prepared according to the method of Example 1 using 1-(2,5-dichlorophenyl)hydrazine hydrochloride in place of o-tolyl-hydrazine hydrochloride in Step A and 2-chloro-3-nitropyridine in place of 1-fluoro-2-nitrobenzene in Step B. ¹H NMR (400 MHz, d-CDCl₃) δ 8.07 (dd, 1H) 7.64 (s, 1H) 7.41 (d, 1H) 7.26-7.36 (m, 2H) 7.11-7.26 (m, 5H) 7.02 (d, 3H) 5.45 (s, 1H) 2.21 (s, 3H)

### EXAMPLE 99

### 1-(4-(1H-pyrazol-1-yl)phenyl)-3-(2-(1-(2-chlorophenyl)-3-methyl-1H-pyrazol-5-yloxy)phenyl)urea

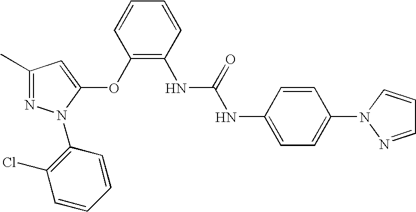

Prepared according to the method of Example 83 [Step D] using 4-(1H-pyrazol-1-yl)benzenamine in place of 2-(4-amino-phenyl)-2-methyl-propionic acid ethyl ester. ¹H NMR (400 MHz, d-DMSO) δ 9.24 (s, 1H) 8.36 (dd, 1H) 8.05 (dd, 1H) 8.21 (s, 1H) 7.54-7.62 (m, 2H) 7.68-7.77 (m, 2H) 7.47-7.53 (m, 2H) 7.38-7.47 (m, 2H) 7.03-7.15 (m, 2H) 6.89-7.00 (m, 1H) 6.47 (dd, 1H) 5.72 (s, 1H) 5.69 (s, 1H) 2.16 (s, 3H)

### EXAMPLE 100

### 1-(4-(1H-imidazol-1-yl)phenyl)-3-(2-(1-(2-chlorophenyl)-3-methyl-1H-pyrazol-5-yloxy)phenyl)urea

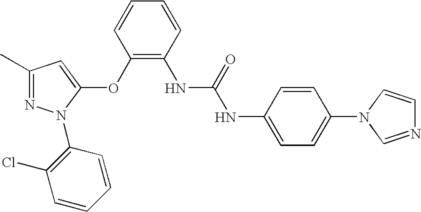

Prepared according to the method of Example 83 [Step D] using 4-(1H-imidazol-1-yl)benzenamine in place of 2-(4-amino-phenyl)-2-methyl-propionic acid ethyl ester. ¹H NMR (400 MHz, d-DMSO-) δ 9.29 (s, 1H) 8.23 (s, 1H) 8.18 (s, 1H) 8.04 (dd, 1H) 7.65 (s, 1H) 7.55-7.61 (m, 2H) 7.52 (s, 3H) 7.40-7.45 (m, 2H) 7.04-7.13 (m, 3H) 6.88-7.00 (m, 1H) 5.69 (s, 1H) 2.16 (s, 3H)

### EXAMPLE 101

### 1-(4-cyanophenyl)-3-(2-(1-(2-ethylphenyl)-3-methyl-1H-pyrazol-5-yloxy)phenyl)urea

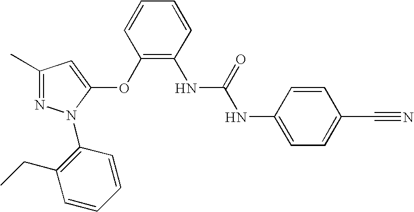

Prepared according to the method of Example 91 substituting 4-aminobenzonitrile in place of 4-phenylaniline in Step D of this procedure. ¹H NMR (400 MHz, d-DMSO-) δ ppm 9.57 (s, 1H) 8.34 (s, 1H) 7.91-8.01 (m, 1H) 7.66-7.76 (m, 2H) 7.52-7.60 (m, 2H) 7.26-7.38 (m, 3H) 7.15-7.23 (m, 1H) 7.03-7.10 (m, 1H) 6.93-7.02 (m, 2H) 5.71 (s, 1H) 2.44-2.50 (m, 2H) 2.16 (s, 3H) 1.01 (t, 3H)

### EXAMPLE 102

### 1-(2-(1-(2-ethylphenyl)-3-methyl-1H-pyrazol-5-yloxy)phenyl)-3-(4-(oxazol-5-yl)phenyl)urea

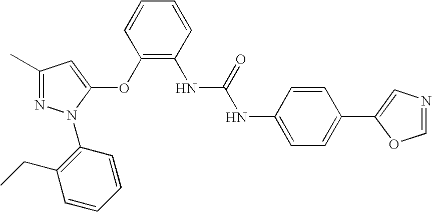

Prepared according to the method of Example 91 substituting 4-(oxazol-5-yl)benzenamine in place of 4-phenylaniline in Step D of this procedure. ¹H NMR (400 MHz, d-DMSO) δ 9.30 (s, 1H) 8.34 (s, 1H) 8.22 (s, 1H) 8.00 (dd, 1H) 7.58-7.64 (m, 2H) 7.46-7.53 (m, 3H) 7.29-7.38 (m, 3H) 7.17-7.24 (m, 1H) 7.02-7.09 (m, 1H) 6.90-6.99 (m, 2H) 5.69 (s, 1H) 2.46-2.53 (m, 2H) 2.15 (s, 3H) 1.02 (t, 3H)

### EXAMPLE 103

### 1-(2-(1-(2-ethylphenyl)-3-methyl-1H-pyrazol-5-yloxy)phenyl)-3-(4-methoxyphenyl)urea

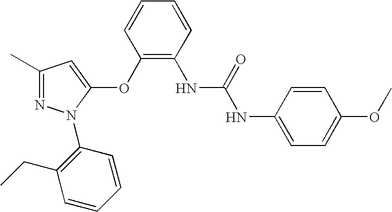

Prepared according to the method of Example 91 substituting 4-methoxybenzenamine in place of 4-phenylaniline in Step D of this procedure. ¹H NMR (400 MHz, d-DMSO) δ 8.93 (s, 1H) 8.06 (s, 1H) 8.01 (dd, 1H) 7.35 (d, 1H) 7.25-7.33 (m, 4H) 7.18-7.24 (m, 1H) 6.99-7.06 (m, 1H) 6.86-6.96 (m, 2H) 6.79-6.85 (m, 2H) 5.66 (s, 1H) 3.67 (s, 3H) 2.46-2.52 (m, 2H) 2.14 (s, 3H) 1.02 (t, 3H)

### EXAMPLE 104

### 1-(2-(1-(2-ethylphenyl)-3-methyl-1H-pyrazol-5-yloxy)phenyl)-3-(4-isopropylphenyl)urea

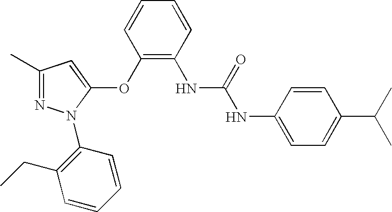

Prepared according to the method of Example 91 substituting 4-isopropylbenzenamine in place of 4-phenylaniline in Step D of this procedure. ¹H NMR (400 MHz, d-DMSO) δ 9.02 (s, 1H) 8.12 (s, 1H) 8.02 (dd, 1H) 7.34-7.39 (m, 1H) 7.26-7.34 (m, 4H) 7.17-7.24 (m, 1H) 7.08-7.14 (m, 2H) 7.00-7.06 (m, 1H) 6.87-6.96 (m, 2H) 5.67 (s, 1H) 2.73-2.84 (m, 1H) 2.46-2.53 (m, 2H) 2.15 (s, 3H) 1.14 (d, 6H) 1.02 (t, 3H)

### EXAMPLE 105

### 1-(2-(1-(2-ethylphenyl)-3-methyl-1H-pyrazol-5-yloxy)phenyl)-3-(4-(piperidin-1-ylmethyl)phenyl)urea

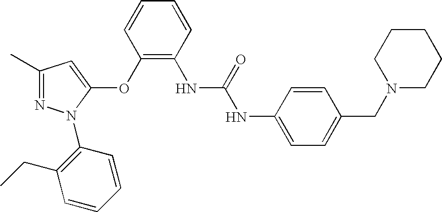

Prepared according to the method of Example 91 substituting 4-(piperidin-1-ylmethyl)benzenamine in place of 4-phenylaniline in Step D of this procedure. ¹H NMR (400 MHz, d-DMSO) δ 9.09 (br. s., 1H) 8.15 (s, 1H) 8.00 (dd, 1H) 7.28-7.38 (m, 4H) 7.11-7.23 (m, 5H) 7.00-7.07 (m, 1H) 6.88-6.97 (m, 2H) 5.67 (s, 1H) 2.46-2.53 (m, 2H) 2.25 (br. s., 4H) 2.14 (s, 3H) 1.44 (br. s., 4H) 1.34 (br. s., 2H) 1.02 (t, 3H)

### EXAMPLE 106

### 1-(2,4-difluorophenyl)-3-(2-(1-(2-ethylphenyl)-3-methyl-1H-pyrazol-5-yloxy)phenyl)urea

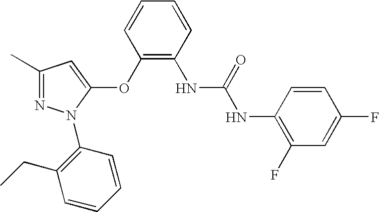

Prepared according to the method of Example 91 substituting 2,4-difluorobenzenamine in place of 4-phenylaniline in Step D of this procedure. ¹H NMR (400 MHz, d-DMSO) δ 8.97 (d, J=1.75 Hz, 1H) 8.60 (s, 1H) 7.96-8.07 (m, 2H) 7.30-7.37 (m, 3H) 7.17-7.29 (m, 2H) 6.95-7.08 (m, 2H) 6.90-6.95 (m, 2H) 5.69 (s, 1H) 2.46-2.53 (m, 2H) 2.15 (s, 3H) 1.02 (t, J=7.60 Hz, 3H)

### EXAMPLE 107

### 1-(2-(1-(2-ethylphenyl)-3-methyl-1H-pyrazol-5-yloxy)phenyl)-3-(4-(trifluoromethyl)phenyl)urea

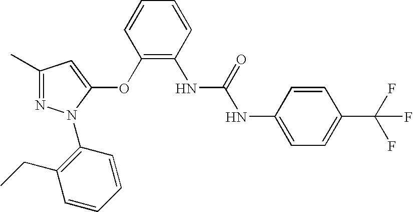

Prepared according to the method of Example 91 substituting 4-(trifluoromethyl)benzenamine in place of 4-phenylaniline in Step D of this procedure. ¹H NMR (400 MHz, d-DMSO) δ 9.48 (s, 1H) 8.27 (s, 1H) 7.96-8.00 (m, 1H) 7.59 (s, 4H) 7.29-7.36 (m, 4H) 7.15-7.22 (m, 1H) 7.02-7.09 (m, 1H) 6.92-6.99 (m, 2H) 5.70 (s, 1H) 2.46-2.53 (m, 2H) 2.15 (s, 3H) 1.01 (t, 3H)

### EXAMPLE 108

### 1-(4-cyclohexylphenyl)-3-(2-(1-(2-ethylphenyl)-3-methyl-1H-pyrazol-5-yloxy)phenyl)urea

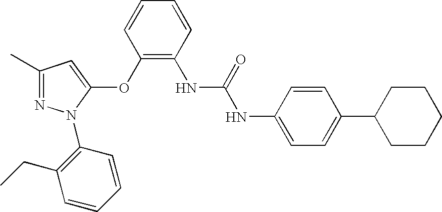

Prepared according to the method of Example 91 substituting 4-cyclohexylbenzenamine in place of 4-phenylaniline in Step D of this procedure. ¹H NMR (400 MHz, d-DMSO) δ 9.00 (s, 1H) 8.11 (s, 1H) 8.01 (dd, 1H) 7.25-7.38 (m, 5H) 7.16-7.24 (m, 1H) 6.98-7.12 (m, 3H) 6.86-6.96 (m, 2H) 5.70 (s, 1H) 5.67 (s, 1H) 2.46-2.51 (m, 2H) 2.14 (s, 3H) 1.56-1.85 (m, 6H) 1.21-1.41 (m, 5H) 1.02 (t, 3H)

### EXAMPLE 109

### 1-(2-(1-(2-ethylphenyl)-3-methyl-1H-pyrazol-5-yloxy)phenyl)-3-(4-isopropoxyphenyl)urea

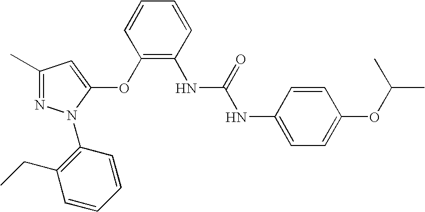

Prepared according to the method of Example 91 substituting 4-isopropoxybenzenamine in place of 4-phenylaniline in Step D of this procedure. ¹H NMR (400 MHz, d-DMSO) δ 8.91 (s, 1H) 8.06 (s, 1H) 8.00 (dd, 1H) 7.30-7.37 (m, 2H) 7.24-7.28 (m, 2H) 7.18-7.24 (m, 1H) 7.00-7.07 (m, 1H) 6.86-6.95 (m, 2H) 6.77-6.83 (m, 2H) 5.66 (s, 1H) 4.46 (dt, 1H) 2.48-2.56 (m, 2H) 2.14 (s, 3H) 1.19 (d, 6H) 1.02 (t, 3H)

### EXAMPLE 110

### 1-(2-(1-(2-ethylphenyl)-3-methyl-1H-pyrazol-5-yloxy)phenyl)-3-(4-(piperidin-1-yl)phenyl)urea

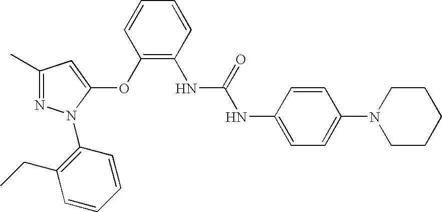

Prepared according to the method of Example 91 substituting 4-(piperidin-1-yl)benzenamine in place of 4-phenylaniline in Step D of this procedure. ¹H NMR (400 MHz, d-DMSO) δ 8.85 (s, 1H) 7.99-8.07 (m, 2H) 7.31-7.39 (m, 2H) 7.21 (d, 2H) 7.00-7.07 (m, 1H) 6.86-6.96 (m, 2H) 6.83 (d, 2H) 5.67 (s, 1H) 2.91-3.06 (m, 3H) 2.45-2.54 (m, 2H) 2.15 (s, 3H) 1.53-1.63 (m, 4H) 1.37-1.52 (m, 2H) 1.03 (t, 3H)

### EXAMPLE 111

### 1-(4-chlorophenyl)-3-(2-(1-(2-ethylphenyl)-3-methyl-1H-pyrazol-5-yloxy)phenyl)urea

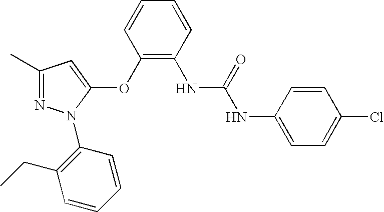

Prepared according to the method of Example 91 substituting 4-chloroaniline in place of 4-phenylaniline in Step D of this procedure. ¹H NMR (400 MHz, d-DMSO) δ 9.24 (s, 1H) 8.19 (s, 1H) 7.98 (d, 1H) 7.42 (d, 2H) 7.26-7.37 (m, 4H) 7.16-7.25 (m, 1H) 7.01-7.10 (m, 1H) 6.90-7.00 (m, 2H) 5.69 (s, 1H) 2.48-2.58 (m, 2H) 1.02 (t, 3H)

### EXAMPLE 112

### 1-(4-(1H-1,2,4-triazol-1-yl)phenyl)-3-(2-(1-(2-ethylphenyl)-3-methyl-1H-pyrazol-5-yloxy)phenyl)urea

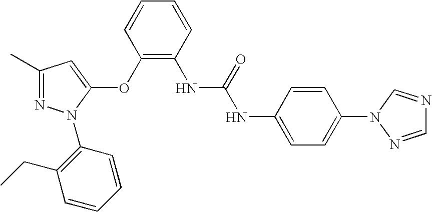

Prepared according to the method of Example 91 substituting 4-(1H-1,2,4-triazol-1-yl)benzenamine in place of 4-phenylaniline in Step D of this procedure. ¹H NMR (400 MHz, d-DMSO) δ 9.34 (s, 1H) 9.15 (s, 1H) 8.24 (s, 1H) 8.16 (s, 1H) 8.01 (dd, 1H) 7.70-7.76 (m, 2H) 7.53-7.60 (m, 2H) 7.28-7.39 (m, 3 H) 7.18-7.25 (m, 1H) 7.04-7.10 (m, 1H) 6.92-7.00 (m, 2H) 5.70 (s, 1H) 2.47-2.53 (m, 2H) 2.16 (s, 3H) 1.03 (t, 3H)

### EXAMPLE 113

### 1-(2-(1-(2-isopropylphenyl)-3-methyl-1H-pyrazol-5-yloxy)phenyl)-3-(4-(trifluoromethoxy)phenyl)urea

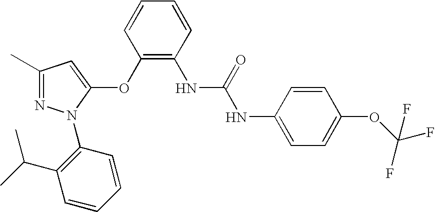

Prepared according to the method of Example 1 using 2-isopropyllphenyl hydrazine hydrochloride in place of o-tolyl-hydrazine hydrochloride in Step A and 2-chloro-3-nitropyridine in place of 1-fluoro-2-nitrobenzene in Step B. ¹H NMR (400 MHz, d-CDCl₃) δ 8.05 (dd, 1H) 7.33-7.42 (m, 3H) 7.28 (d, 1H) 7.19-7.24 (m, 2H) 7.07-7.17 (m, 3H) 6.98-7.06 (m, 3H) 5.55 (s, 1H) 3.27-3.43 (m, 1H) 2.68-2.91 (m, 2H) 2.18 (s, 3H) 1.13 (d, 6H)

### EXAMPLE 114

### 1-(2-(1-(2-chloro-4-fluorophenyl)-3-methyl-1H-pyrazol-5-yloxy)phenyl)-3-(4-(trifluoromethoxy)phenyl)urea

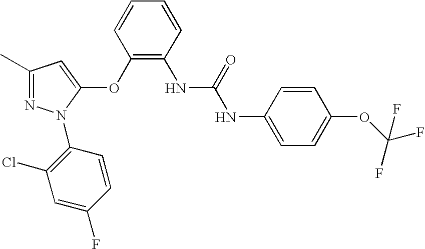

Prepared according to the method of Example 1 using 1-(2-chloro-4-fluorophenyl)hydrazine hydrochloride in place of o-tolyl-hydrazine hydrochloride in Step A and 2-chloro-3-nitropyridine in place of 1-fluoro-2-nitrobenzene in Step B. ¹H NMR (400 MHz, d-DMSO) δ 9.27 (s, 1H) 8.21 (s, 1H) 8.14 (d, 1H) 7.57-7.62 (m, 2H) 7.48-7.50 (m, 2H) 7.33-7.21 (m, 3H) 7.04-7.10 (m, 2H) 6.89-6.98 (m, 1H) 5.70 (s, 1H) 2.15 (s, 3H)

### EXAMPLE 115

### 1-(2-(1-(2-chloro-4-fluorophenyl)-3-methyl-1H-pyrazol-5-yloxy)phenyl)-3-(4-cyclohexylphenyl)urea

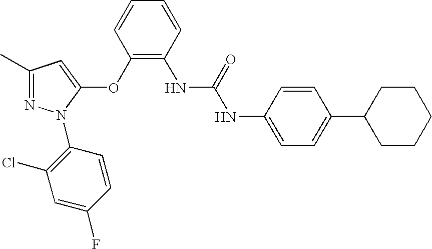

Prepared from 2-(1-(2-chloro-4-fluorophenyl)-3-methyl-1H-pyrazol-5-yloxy)benzenamine [based on the method of Example 1 using 1-(2-chloro-4-fluorophenyl)hydrazine hydrochloride in place of o-tolyl-hydrazine hydrochloride in Step A and 2-chloro-3-nitropyridine in place of 1-fluoro-2-nitrobenzene in Step B] and 4-cyclohexylaniline using the method of Example 83 [Step C and D] in place of 2-(4-amino-phenyl)-2-methyl-propionic acid ethyl ester. ¹H NMR (400 MHz, d-DMSO) δ 9.11 (s, 1H) 8.17 (s, 1H) 7.98-8.12 (m, 1H) 7.54-7.60 (m, 2H) 7.38-7.48 (m, 2H) 7.33-7.39 (m, 2H) 7.14-7.21 (m, 2H) 7.04-7.10 (m, 2H) 6.89-6.98 (m, 1H) 5.68 (s, 1H) 4.20 (q, 1H) 3.05 (s, 3H) 2.16 (s, 3H) 1.28 (d, 3H)

### EXAMPLE 116

### 1-(2-(1-(2-chlorophenyl)-3-methyl-1H-pyrazol-5-yloxy)phenyl)-3-(4-(1-methoxyethyl)phenyl)urea

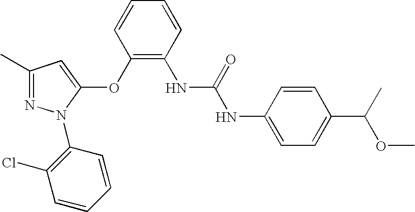

### Step A

### 1-(4-nitrophenyl)ethanol

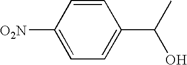

To a solution of 4-nitroacetophenone (5.0 g, 30.3 mmol) in THF:MeOH (1:1, 50 mL) at 0° C. was added NaBH₄ (1.26 g, 33.3 mmol). The reaction mixture was stirred at 0° C. for 30 min and then at 25° C. for an additional 30 min. Subsequently, the reaction mixture was quenched by addition of sat. NH₄Cl solution (10 mL) followed by 1 N HCl (25 mL) and ethyl acetate (100 mL). The organic layer was separated, dried and concentrated to afford 1-(4-nitrophenyl)ethanol (4.58 g, 91%) as a colorless oil. ¹H NMR (400 MHz, CDCl₃) δ 8.17 (d, 2H), 7.51 (d, 2H), 4.99 (q, 1H), 2.11-2.06 (m, 1H), 1.50 (d, 3H).

### Step B

### 1-(1-methoxyethyl)-4-nitrobenzene

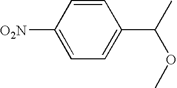

To a solution of 1-(4-nitrophenyl)ethanol (2.0 g, 12.0 mmol) in THF at 0° C. was added sodium hydride (502 mg, 12.6 mmol). The reaction mixture was stirred at 0° C. for 15 min after which time iodomethane (1.5 ml, 23.9 mmol) was added and the reaction was allowed to warm to 25° C. and stirred for 2 hrs. The reaction mixture was quenched by slow addition of sat. NH₄Cl solution and ethyl acetate (75 mL) was then added. The organic layer was separated, dried and concentrated to a yellow oil that was purified by column chromatography (5% EtOAc/Hex) to afford 1-(1-methoxyethyl)-4-nitrobenzene (1.25 g, 58%). ¹H NMR (400 MHz, CDCl₃) δ 8.21 (d, 2H), 7.45 (d, 2H), 4.40 (q, 1H), 3.23 (s, 3H), 1.41 (d, 3H).

### Step C

### 4-(1-methoxyethyl)benzenamine

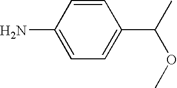

To a solution of 1-(1-methoxyethyl)-4-nitrobenzene (1.25 g, 6.90 mmol) in MeOH (50 mL) under a nitrogen atmosphere was added Raney Nickel catalyst (100 mg). The reaction vessel was evacuated, flushed with nitrogen and then filled with hydrogen (50 psi). The reaction mixture was agitated at 25° C. for 1.5 hrs after which time the reaction vessel was flushed with nitrogen and filtered through a pad of celite. The filtrate was concentrated to afford 4-(1-methoxyethyl)benzenamine (1.0 g, 96%) as a white solid. ¹H NMR (400 MHz, CDCl₃) δ 7.08 (d, 2H), 6.68 (d, 2H), 4.16 (q, 1H), 3.78 (bs, 2H), 3.17 (s, 3H), 1.39 (d, 3H).

### Step D

### 1-(2-(1-(2-chlorophenyl)-3-methyl-1H-pyrazol-5-yloxy)phenyl)-3-(4-(1-methoxyethyl)phenyl)urea

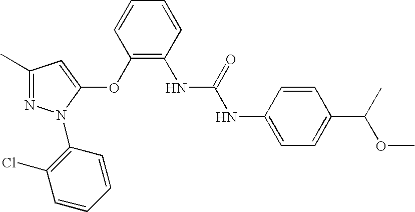

Prepared from the reaction of 4-(1-methoxyethyl)benzenamine and 1-(2-chloro-phenyl)-5-(2-isocyanato-phenoxy)-3-methyl-1H-pyrazole (from Example 83) according to the method of Example 83. ¹H NMR (400 MHz, d-DMSO) δ 9.11 (s, 1H) 8.17 (s, 1H) 7.98-8.12 (m, 1H) 7.54-7.60 (m, 2H) 7.38-7.48 (m, 2H) 7.33-7.39 (m, 2H) 7.14-7.21 (m, 2H) 7.04-7.10 (m, 2H) 6.89-6.98 (m, 1H) 5.68 (s, 1H) 4.20 (q, 1H) 3.05 (s, 3H) 2.16 (s, 3H) 1.28 (d, 3H)

### EXAMPLE 117

### 1-(2-(1-(2-chlorophenyl)-3-methyl-1H-pyrazol-5-yloxy)phenyl)-3-(4-(1-(dimethylamino)ethyl)phenyl)urea

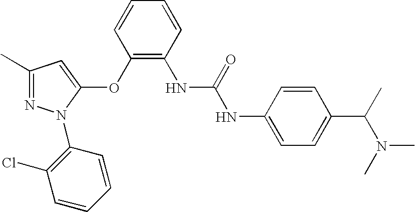

### Step A

### 1-(1-bromoethyl)-4-nitrobenzene

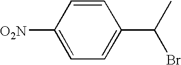

A solution of 1-ethyl-4-nitrobenzene (10.0 g, 66 mmol), N-bromosuccinamide (11.8 g, 66 mmol) and benzoyl peroxide (160 mg, 0.66 mmol) in CCl₄ (100 (mL) was heated to reflux for 1.5 hrs. The reaction mixture was then cooled to 0° C. and filtered through a fritted funnel. The isolated solid was washed once with hexane (50 mL) and the combined filtrates were concentrated and purified by column chromatography (2-5% EtOAc/Hex) to afford 1-(1-bromoethyl)-4-nitrobenzene (7.52 g, 49%). ¹H NMR (400 MHz, CDCl₃) δ 8.21 (d, 2H), 7.58 (d, 2H), 5.22 (q, 1H), 2.05 (d, 3H).

### Step B

### N,N-dimethyl-1-(4-nitrophenyl)ethanamine

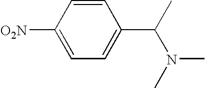

To a solution of 1-(1-bromoethyl)-4-nitrobenzene (1.3 g, 5.65 mmol) in DMF (10 mL) was added dimethyl amine (14.1 mL of 2.0 M solution in THF, 28.3 mmol) and potassium carbonate (2.34 g, 17 mmol). Reaction was stirred at 25° C. for 17 hrs. Subsequently, EtOAc (50 mL) and water (50 mL) were added. The organic layer was separated, washed with brine and concentrated to afford N,N-dimethyl-1-(4-nitrophenyl)ethanamine (0.98 g, 89%). ¹H NMR (400 MHz, CDCl₃) δ 8.18 (d, 2H), 7.51 (d, 2H), 3.38 (q, 1H), 2.10 (s, 6H), 1.36 (d, 3H).

### Step C

### 4-(1-(dimethylamino)ethyl)benzenamine

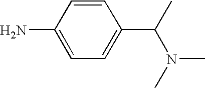

To a solution of 1-(1-methoxyethyl)-4-nitrobenzene (0.98 g, 5.00 mmol) in MeOH (50 mL) under a nitrogen atmosphere was added Raney nickel catalyst (100 mg). The reaction vessel was evacuated, flushed with nitrogen and then filled with hydrogen (50 psi). The reaction mixture was agitated at 25° C. for 1.5 hrs after which time the reaction vessel was flushed with nitrogen and filtered through a pad of celite. The filtrate was concentrated and purified by column chromatography (15% MeOH, 79% CH₂Cl₂, 1% NH₄OH) to afford 4-(1-(dimethylamino)ethyl)benzenamine (0.33 g, 40%). ¹H NMR (400 MHz, CDCl₃) δ 7.04 (d, 2H), 6.79 (d, 2H), 3.59 (bs, 2H), 3.18 (q, 1H), 2.18 (s, 6H), 1.35 (d, 3H).

### Step D

### 1-(2-(1-(2-chlorophenyl)-3-methyl-1H-pyrazol-5-yloxy)phenyl)-3-(4-(1-(dimethylamino)ethyl)phenyl)urea

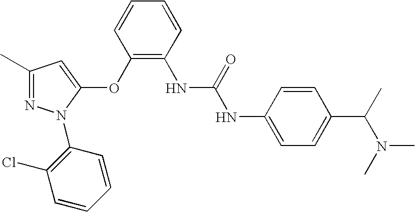

Prepared from the reaction of 4-(1-(dimethylamino)ethyl)benzenamine and 1-(2-chloro-phenyl)-5-(2-isocyanato-phenoxy)-3-methyl-1H-pyrazole (from Example 83) according to the method of Example 83. ¹H NMR (400 MHz, d-DMSO) δ 9.49 (s, 1H) 8.32 (s, 1H) 8.03 (dd, 1H) 7.81-7.92 (m, 2H) 7.48-7.60 (m, 3H) 7.37-7.46 (m, 2H) 7.05-7.12 (m, 2H) 6.94-7.01 (m, 1H) 5.70 (s, 1H) 3.99 (q, 1H) 2.48 (s, 3H) 2.16 (s, 3H).

### EXAMPLE 118

### 1-(4-acetylphenyl)-3-(2-(1-(2-chlorophenyl)-3-methyl-1H-pyrazol-5-yloxy)phenyl)urea

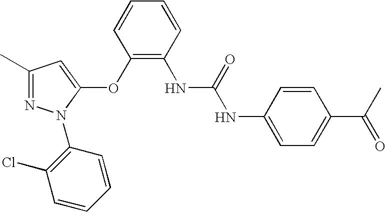

Prepared according to the method of Example 83 [Step D] using 1-(4-aminophenyl)ethanone in place of 2-(4-amino-phenyl)-2-methyl-propionic acid ethyl ester. ¹H NMR (400 MHz, d-DMSO) δ 9.49 (s, 1H) 8.32 (s, 1H) 8.03 (dd, 1H) 7.81-7.92 (m, 2H) 7.48-7.60 (m, 3H) 7.37-7.46 (m, 2H) 7.05-7.12 (m, 2H) 6.94-7.01 (m, 1H) 5.70 (s, 1H) 2.48 (s, 3H) 2.16 (s, 3H)

### EXAMPLE 119

### 1-(2-(1-(2-propylphenyl)-3-methyl-1H-pyrazol-5-yloxy)phenyl)-3-(4-(trifluoromethoxy)phenyl)urea

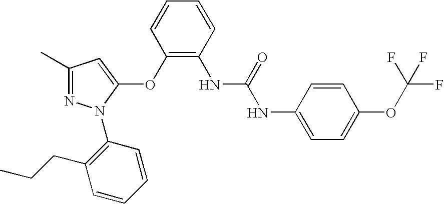

Prepared according to the method of Example 1 using 2-n-propylphenyl hydrazine hydrochloride in place of o-tolyl-hydrazine hydrochloride in Step A and 2-chloro-3-nitropyridine in place of 1-fluoro-2-nitrobenzene in Step B. ¹H NMR (400 MHz, d-CDCl₃) δ 8.00-8.06 (m, 1H) 7.71 (s, 1H) 7.21-7.30 (m, 2H) 7.13-7.20 (m, 2H) 7.08-7.13 (m, 3H) 6.96-7.04 (m, 3H) 5.49 (s, 1H) 2.38-2.58 (m, 2H) 2.20 (s, 3H) 1.39-1.55 (m, 2H) 0.83 (t, 3H)

### EXAMPLE 120

### 1-(2-(1-(2-chlorophenyl)-3-methyl-1H-pyrazol-5-yloxy)phenyl)-3-(4-(piperidin-4-yl)phenyl)urea

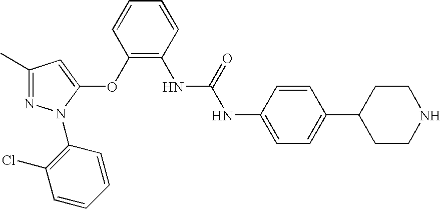

### Step A

### tert-butyl 4-(4-aminophenyl)piperidine-1-carboxylate

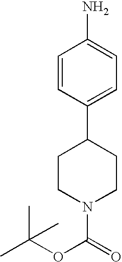

A mixture of 4-pyridyl-nitrobenzene (1.0 g, 5.0 mmol), PtO₂ (0.1 g) in 0.77 N HCl (25 mL) was treated with hydrogen gas in a Parr shaker at 25° C. for 7 days. Subsequently, the reaction vessel was purged with nitrogen and the reaction mixture was filtered through celite and the filtrate was concentrated in vacuo to give 4-(piperidin-4-yl)benzenamine hydrochloride (1.23 g, 99%) as a solid that was utilized without additional purification. To 4-(piperidin-4-yl)benzenamine hydrochloride (1.23 g, 5.0 mmol) in THF (50 ml) at 25° C. was added triethyl amine (2.54 g, 25.1 mmol) and the resulting solution was stirred at 25° C. for 30 min, then cooled at 0° C. Subsequently, di-tert-butyl dicarbonate (1.09 g, 5.02 mmol) was added in portions. The resulting solution was monitored by HPLC. After the reaction was completed, the reaction mixture was diluted with EtOAc (50 mL) and water (25 mL) was added. The organic layer was separated, washed with sat. NaHCO₃ solution, water and brine, dried over Na₂SO₄, filtered, and concentrated. The crude product was purified by column chromatography (15-40% EtOAc/Heptane) to give tert-butyl 4-(4-aminophenyl)piperidine-1-carboxylate (0.72 g, 51%) as a light red oil. ¹H NMR (400 MHz, d-CDCl₃) δ 6.97-7.03 (m, 2H) 6.68-6.74 (m, 2H) 4.20 (br. s., 2H) 2.70-2.87 (m, 2H) 2.47-2.60 (m, 2H) 1.71-1.81 (m, 2H) 1.50-1.61 (m, 2H) 1.46 (s, 9H)

### Step B

### 1-(2-(1-(2-chlorophenyl)-3-methyl-1H-pyrazol-5-yloxy)phenyl)-3-(4-(piperidin-4-yl)phenyl)urea

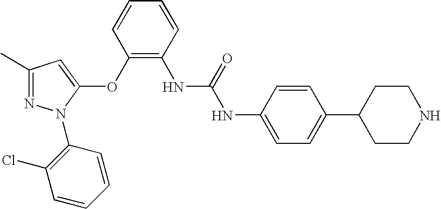

A portion of tert-butyl 4-(4-aminophenyl)piperidine-1-carboxylate (764 mg, 2.76 mmol) and 1-(2-chloro-phenyl)-5-(2-isocyanato-phenoxy)-3-methyl-1H-pyrazole [from Example 83 Step C] (900 mg, 2.76 mmol) was mixed in THF (15 ml) and triethyl amine (839 mg, 8.29 mmol) was added. The reaction was heated at 60° C. for 14 hrs. The crude product was purified by column chromatography (20-50% EtOAc/Heptane to afford tert-butyl 4-(4-(3-(2-(1-(2-chlorophenyl)-3-methyl-1H-pyrazol-5-yloxy)phenyl)ureido)phenyl)piperidine-1-carboxylate (1.66 g, 78%). To tert-butyl 4-(4-(3-(2-(1-(2-chlorophenyl)-3-methyl-1H-pyrazol-5-yloxy)phenyl)ureido)phenyl)piperidine-1-carboxylate (1.66 g, 2.75 mmol) in CH₂Cl₂ (15 mL), trifluoroacetic acid (5 ml) was added at 25° C. and the reaction mixture was stirred for 3 hrs. The reaction mixture was then concentrated under reduced pressure, CH₂Cl₂ (50 mL) was added and the organic layer was washed with aqueous sodium bicarbonate, washed with brine, dried over Na₂SO₄, filtered, and concentrated. The crude product was purified by column chromatography (10% MeOH/CH₂Cl₂, 0.1% ammonium hydroxide) to afford the title compound as a white solid (1.25 g, 68% for 2 steps). ¹H NMR (400 MHz, d-DMSO) δ 8.32 (s, 1H) 8.04 (dd, 1H) 7.67 (dd, 1H) 7.54-7.59 (m, 1H) 7.36-7.47 (m, 2H) 7.28-7.35 (m, 1H) 7.00-7.11 (m, 3H) 6.85-6.97 (m, 1H) 5.64 (s, 1H) 2.89-3.04 (m, 3H) 2.49-2.69 (m, 2H) 2.14 (s, 3H) 1.62 (dd, 2H) 1.31-1.53 (m, 2H)

### EXAMPLE 121

### 1-(4-(1-acetylpiperidin-4-yl)phenyl)-3-(2-(1-(2-chlorophenyl)-3-methyl-1H-pyrazol-5-yloxy)phenyl)urea

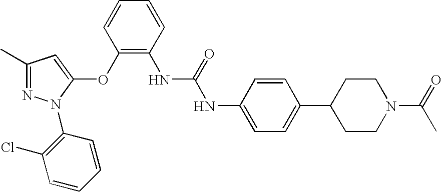

To 1-(2-(1-(2-chlorophenyl)-3-methyl-1H-pyrazol-5-yloxy)phenyl)-3-(4-(piperidin-4-yl)phenyl)urea [from Example 120] (115 mg, 0.23 mmol) in CH₂Cl₂ (20 ml) at 25° C. was added triethylamine (70 mg, 0.69 mmol) was added, followed by acetic anhydride (26 mg, 0.25 mmol). The reaction mixture was stirred at 25° C. for 14 hrs. The reaction mixture was then concentrated. The crude product was purified by column chromatography (90% EtOAc/Heptane) to afford the title compound as a white solid (122 mg, 98%). H NMR (400 MHz, d-DMSO) δ 8.53 (s, 1H) 8.04 (d, 1H) 7.79 (dd, 1H) 7.54 (dd, 1H) 7.30-7.44 (m, 3H) 6.97-7.13 (m, 3H) 6.86-6.95 (m, 1H) 5.60 (s, 1H) 4.47 (d, 2H) 3.85 (d, 2H) 2.97-3.16 (m, 2H) 2.59-2.73 (m, 1H) 2.54 (d, 2H) 2.13 (s, 3H) 1.98 (s, 3H) 1.72 (t, 2H) 1.43-1.58 (m, 2H) 1.24-1.40 (m, 2H)

### EXAMPLE 122

### 1-(2-(1-(2-chlorophenyl)-3-methyl-1H-pyrazol-5-yloxy)phenyl)-3-(4-(1-methylpiperidin-4-yl)phenyl)urea

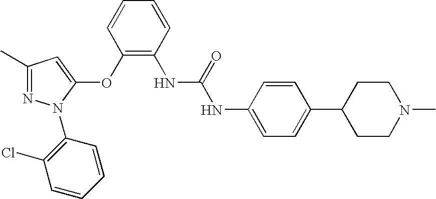

To a solution of 1-(2-(1-(2-chlorophenyl)-3-methyl-1H-pyrazol-5-yloxy)phenyl)-3-(4-(piperidin-4-yl)phenyl)urea [from Example 120] (150 mg) in DMF (20 ml) at 25° C. was added potassium carbonate (124 mg, 0.90 mmol) and iodomethane (18.6 ul, 0.60 mmol). The reaction was stirred at 25° C. for 14 hr. The reaction mixture was then diluted with EtOAc and filtered to remove excess potassium acetate. The filtrate was washed with water and brine, dried (Na₂SO₄), filtered and concentrated. The crude product was purified by column chromatography (10% MeOH/CH₂Cl₂, 1% ammonium hydroxide) to afford title compound (22 mg, 30%). ¹H NMR (400 MHz, d-DMSO) δ 8.16 (s, 1H) 8.04 (dd, 1H) 7.52-7.63 (m, 2H) 7.38-7.47 (m, 2H) 7.35 (d, 2H) 7.24 (d, 2H) 7.03-7.12 (m, 2H) 6.88-6.97 (m, 2H) 5.67 (s, 1H) 3.33-3.57 (m, 3H) 3.12 (s, 3H) 2.69 (d, 2H) 2.15 (s, 3H) 1.97-2.06 (m, 2H) 1.90 (br. s., 2H)

### EXAMPLE 123

### 1-(2-(1-(2-chlorophenyl)-3-methyl-1H-pyrazol-5-yloxy)phenyl)-3-(4-cyclopropylphenyl)urea

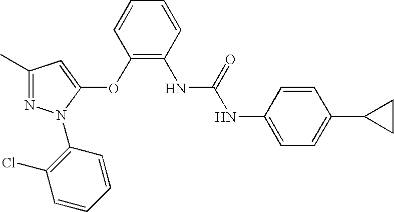

### Step A

### 1-cyclopropyl-4-nitrobenzene

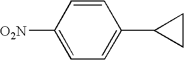

To a solution of cyclopropylbenzene (10.0 g, 66 mmol) in acetic anhydride (50 mL) at 0° C. was slowly added HNO₃ (6.35 mL). The reaction mixture was then allowed to warm to 25° C. and stirred for 4 hrs. Subsequently, the reaction mixture was poured onto ice and ether (200 mL) was added. The organic layer was separated, dried and concentrated to an orange oil that was purified by column chromatography (2-5% EtOAc/Hex) to afford a 1:1 inseparable mixture of 1-cyclopropyl-4-nitrobenzene and 1-cyclopropyl-2-nitrobenzene (7.0 g) which was utilized in the next step

### Step B

### 4-cyclopropylbenzenamine

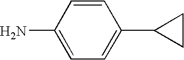

A vessel containing a solution of a 1:1 mixture of 1-cyclopropyl-4-nitrobenzene and 1-cyclopropyl-2-nitrobenzene (7.0 g total) in MeOH (100 mL) was evacuated, flushed with nitrogen and Pd—C (150 mg) was then added. The vessel was again evacuated, filled with hydrogen (via balloon) and stirred at RT for 4 hrs after which time HPLC analysis indicated that starting material was consumed. Flask was flushed with nitrogen and catalyst was removed via filtration through a pad of celite. The filtrate was concentrated to a pink oil that was purified by column chromatography (10-20% EtOAc/Hex) to afford 4-cyclopropylaniline (1.35 g) as the lower Rf fraction. ¹H NMR (400 MHz, CDCl₃) δ 6.87 (d, 2H), 6.57 (d, 2H), 3.45 (bs, 2H), 1.79-1.57 (m, 1H), 0.84-0.82 (m, 2H), 0.56-0.52 (m, 2H).

### Step C

### 1-(2-(1-(2-chlorophenyl)-3-methyl-1H-pyrazol-5-yloxy)phenyl)-3-(4-cyclopropylphenyl)urea

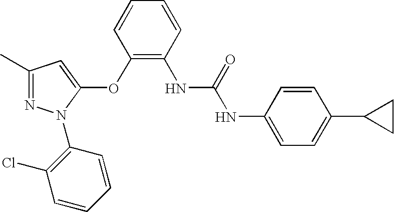

Prepared from the reaction of 4-cyclopropylbenzenamine and 1-(2-chloro-phenyl)-5-(2-isocyanato-phenoxy)-3-methyl-1H-pyrazole (from Example 83) according to the method of Example 83. ¹H NMR (400 MHz, d-DMSO) δ 9.00 (s, 1H) 8.12 (s, 1H) 8.05 (dd, 1H) 7.54-7.60 (m, 2H) 7.40-7.45 (m, 2H) 7.23-7.28 (m, 2H) 7.03-7.09 (m, 2H) 6.91-6.98 (m, 3H) 5.67 (s, 1H) 2.15 (s, 3H) 1.75-1.86 (m, 1H) 0.80-0.94 (m, 2H) 0.49-0.64 (m, 2H)

### EXAMPLE 124

### 1-(2-(1-(2-chlorophenyl)-3-methyl-1H-pyrazol-5-yloxy)phenyl)-3-(4-cyclopentylphenyl)urea

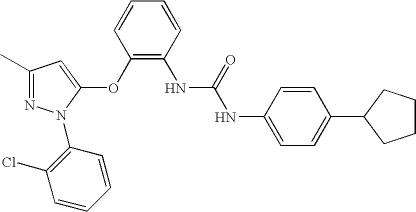

### Step A

### 1-cyclopentylbenzene

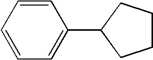

To a suspension of AlCl₃ (15.5 g, 116 mmol) in benzene (100 mL) at 25° C. was slowly added cyclopentanol (10.5 mL, 116 mL). The reaction mixture was stirred at 25° C. for 72 hrs. Subsequently, the organic layer was washed with sat. Na₂CO₃ solution, washed with brine, dried and concentrated to an oil that was purified by vacuum distillation (distillation head temp: 60° C.) to afford 1-cyclopentylbenzene (9.70 g, 57%). ¹H NMR (400 MHz, CDCl₃) δ 7.24-7.21 (m, 5H), 2.99-2.95 (m, 1H), 2.07-2.03 (m, 2H), 1.81-1.49 (m, 6H).

### Step B

### 4-cyclopentylbenzenamine

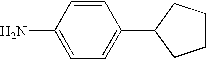

Prepared from 1-cyclopentylbenzene according to the method of Example 123 (Steps A and B). 1H NMR (400 MHz, CDCl₃) δ 7.02 (d, 2H), 6.63 (d, 2H), 3.57 (bs, 1H), 2.88-2.84 (m, 1H), 2.03-2.00 (m, 2H), 1.77-1.49 (m, 6H).

### Step C

### 1-(2-(1-(2-chlorophenyl)-3-methyl-1H-pyrazol-5-yloxy)phenyl)-3-(4-cyclopentylphenyl)urea

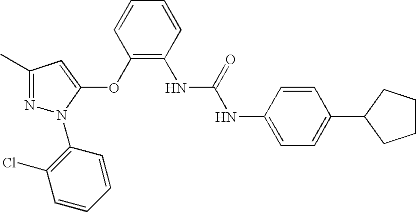

Prepared from the reaction of 4-cyclopentylbenzenamine

and 1-(2-chloro-phenyl)-5-(2-isocyanato-phenoxy)-3-methyl-1H-pyrazole (from Example 83) according to the method of Example 83. 1H NMR (400 MHz, d-DMSO) δ 9.02 (s, 1H) 8.13 (s, 1H) 8.06 (dd, 1H) 7.54-7.65 (m, 2H) 7.39-7.52 (m, 2H) 7.26-7.32 (m, 2H) 7.04-7.14 (m, 3H) 6.85-6.96 (m, 2H) 5.67 (s, 1H) 2.77-2.96 (m, 1H) 2.15 (s, 3H) 1.89-2.00 (m, 2H) 1.66-1.78 (m, 2H) 1.55-1.64 (m, 2H) 1.39-1.51 (m, 2H).

### EXAMPLE 125

### 1-(2-(1-(2-bromophenyl)-3-methyl-1H-pyrazol-5-yloxy)phenyl)-3-(4-(trifluoromethoxy)phenyl)urea

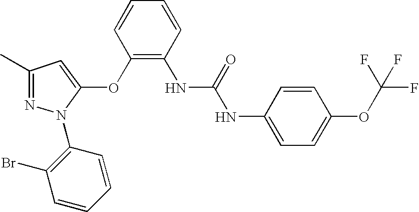

Prepared according to the method of Example 1 using 2-bromohenyl hydrazine hydrochloride in place of o-tolyl-hydrazine hydrochloride in Step A and 2-chloro-3-nitropyridine in place of 1-fluoro-2-nitrobenzene in Step B. ¹H NMR (400 MHz, d-DMSO) δ 9.29 (s, 1H) 8.19 (s, 1H) 8.01 (dd, 1H) 7.73 (dd, 1H) 7.53 (dd, 1H) 7.47-7.50 (m, 2H) 7.42-7.47 (m, 1H) 7.33-7.38 (m, 1H) 7.25 (d, 2H) 7.12 (dd, 1H) 7.06-7.11 (m, 1H) 6.93-6.99 (m, 1H) 5.66 (s, 1H) 2.15 (s, 3H)

### EXAMPLE 126

### 1-(4-bromophenyl)-3-(2-(1-(2-chlorophenyl)-3-methyl-1H-pyrazol-5-yloxy)phenyl)urea

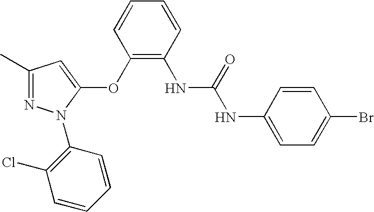

Prepared according to the method of Example 83 [Step D] using 4-bromoaniline in place of 2-(4-amino-phenyl)-2-methyl-propionic acid ethyl ester. ¹H NMR (400 MHz, d-DMSO) δ 9.23 (s, 1H) 8.20 (s, 1H) 8.01 (dd, 1H) 7.51-7.63 (m, 2H) 7.31-7.47 (m, 5H) 7.03-7.12 (m, 2H) 6.90-7.01 (m, 1H) 5.68 (s, 1H) 2.15 (s, 3H)

### EXAMPLE 127

### 1-(2-(1-(2-chlorophenyl)-3-methyl-1H-pyrazol-5-yloxy)phenyl)-3-(4-(tetrahydro-2H-thiopyran-4-yl)phenyl)urea

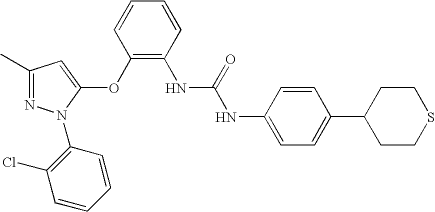

### Step A

### tert-butyl 4-(4-hydroxy-tetrahydro-2H-thiopyran-4-yl)phenylcarbamate

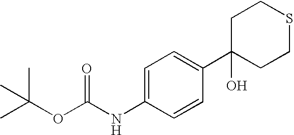

A solution of 4-bromo-N-Boc aniline (6.0 g, 22.1 mmol) in THF (100 ml) at −78° C. under nitrogen was added n-butyllithium (1.6 M in hexane, 34.4 ml, 55 mmol) dropwise over 20 min. The resulting yellow solution was stirred at −78° C. for 30 min and was then treated with a solution of tetrahydrothiopyran-4-one (2.82 g, 24.3 mmol) in THF (25 mL). The reaction mixture was stirred for 4 hrs, during which the reaction temperature was allowed to rise to 0° C. The eraction was quenched with saturated aqueous ammonium chloride (25 ml). The mixture was then diluted with water (25 ml) and Et₂O (25 ml). The layers were separated, and the organic phase was washed with brine (10 ml), dried over Na₂SO₄, and concentrated in vacuo. The residue was flushed through a plug of silica gel with EtOAc:Heptane (1:1) to afford tert-butyl 4-(4-hydroxy-tetrahydro-2H-thiopyran-4-yl)phenylcarbamate (6.2 gram, 92%) as a white solid. ¹H NMR (400 MHz, d-CDCl₃) δ 7.30-7.41 (m, 4H) 6.44 (br. s., 1H) 3.13-3.24 (m, 2H) 2.41-2.50 (m, 2H) 2.09-2.19 (m, 2H) 1.95-2.03 (m, 2H) 1.50 (s, 9H)

### Step B

### 4-(3,6-dihydro-2H-thiopyran-4-yl)benzenamine

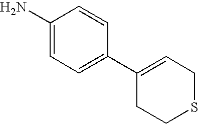

To tert-butyl 4-(4-hydroxy-tetrahydro-2H-thiopyran-4-yl)phenylcarbamate (2.0 g, 6.5 mmol) in CH₂Cl₂ (40 ml), Trifluoroacetic acid (9.6 ml, 129 mmol) was added, followed by drop-wise addition of triethylsilane (5.16 ml, 32.3 mmol) at 25° C. The reaction was completed in 1 hr. The reaction mixture was made basic by addition of aqueous sodium carbonate (saturated) resulting in precipitate formation. The precipitates were removed by filtration and the filtrate was diluted with Et₂O and water was added. The organic layer was separated, washed with water and brine, dried (Na₂SO₄), filtered and concentrated. The crude product was purified by column chromatography (30% ethyl acetate in heptane) to afford white solid (1.18 g, 94%). ¹H NMR (400 MHzd-CDCl3) δ 7.20-7.24 (m, 1H) 6.97-7.16 (m, 2H) 6.90-6.97 (m, 1H) 6.06-6.11 (m, 1H) 3.29-3.34 (m, 1H) 2.76-2.89 (m, 2H) 2.60-2.72 (m, 2H) 2.43-2.53 (m, 1H) 2.04-2.13 (m, 1H) 1.74-1.86 (m, 1H)

### Step C

### 4-(tetrahydro-2H-thiopyran-4-yl)benzenamine

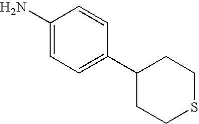

4-(3,6-dihydro-2H-thiopyran-4-yl)benzenamine (1.18 g, 6.2 mmol) in 20 ml of methanol was hydrogenated with platinum oxide (150 mg) in Parr shaker for 4 hrs. The reaction vessel was then purged with nitrogen and the catalyst was filtered off and filtrate was concentrated. The crude product was purified by column chromatography (15% ethyl acetate in Heptane) to give 4-(tetrahydro-2H-thiopyran-4-yl)benzenamine

(1.06 gram, 89%) ¹H NMR (400 MHz, d-DMSO) 66.75-6.86 (m, 2H) 6.39-6.49 (m, 2H) 4.78 (s, 2H) 2.65-2.75 (m, 2H) 2.54-2.62 (m, 2H) 2.25-2.35 (m, 1H) 1.88-1.98 (m, 2H) 1.52-1.65 (m, 2H)

### Step C

### 1-(2-(1-(2-chlorophenyl)-3-methyl-1H-pyrazol-5-yloxy)phenyl)-3-(4-(tetrahydro-2H-thiopyran-4-yl)phenyl)urea

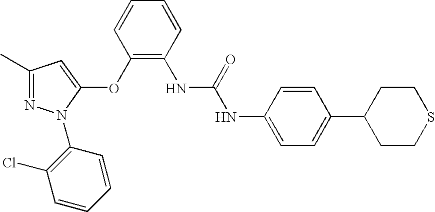

Prepared according to the method of Example 83 [Step D] using 4-(tetrahydro-2H-thiopyran-4-yl)benzenamine in place of 2-(4-amino-phenyl)-2-methyl-propionic acid ethyl ester. ¹H NMR (400 MHz, d-DMSO) δ 9.03 (br. s., 1H) 8.14 (br. s., 1H) 8.05 (d, 1H) 7.57 (t, 2H) 7.37-7.47 (m, 2H) 7.30 (d, 2H) 7.01-7.14 (m, 4H) 6.84-6.99 (m, 1H) 5.67 (s, 1H) 2.74 (t, 2H) 2.61 (d, 2H) 2.15 (s, 3H) 1.98 (d, 2H) 1.57-1.76 (m, 2H)

### EXAMPLE 128

### 1-(2-(1-(2-chlorophenyl)-3-methyl-1H-pyrazol-5-yloxy)phenyl)-3-(4-(tetrahydro-2H-pyran-4-yl)phenyl)urea

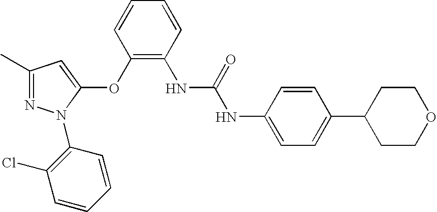

Prepared according to the method of Example 127 using tetrahydropyran-4-one in place of tetrahydrothiopyran-4-one in Step A. ¹H NMR (400 MHz, d-DMSO) δ 9.04 (s, 1H) 8.14 (s, 1H) 8.02-8.08 (m, 1H) 7.53-7.62 (m, 2H) 7.38-7.48 (m, 2H) 7.28-7.34 (m, 2H) 7.03-7.16 (m, 4H) 6.89-6.97 (m, 1H) 5.67 (s, 1H) 3.90 (dd, 2H) 3.33-3.48 (m, 2H) 2.58-2.75 (m, 1H) 2.15 (s, 3H) 1.50-1.72 (m, 4H)

### EXAMPLE 129

### 1-(2-(1-(2-chlorophenyl)-3-methyl-1H-pyrazol-5-yloxy)phenyl)-3-(4-(tetrahydro-2H-oxothiopyran-4-yl)phenyl)urea

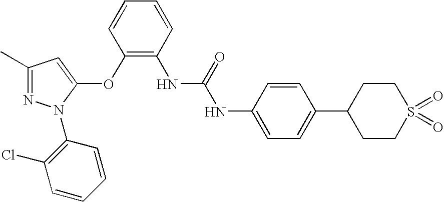

A solution of 1-(2-(1-(2-chlorophenyl)-3-methyl-1H-pyrazol-5-yloxy)phenyl)-3-(4-(tetrahydro-2H-thiopyran-4-yl)phenyl)urea [Example 127] (220 mg, 0.42 mmol) in water:acetone (1:4, 50 ml) was treated with 4-methylmorphorine N-oxide (149 mg) followed by osmium tetraoxide (2.5% wt in t-BuOH, 266 ul). The resulting mixture was stirred at 25° C. for 3.5 hr. The mixture was then quenched with saturated aqueous sodium bisulfite (20 ml), and extracted with CH₂Cl₂ (50 ml) two times. The combined organic phase was washed with brine, dried over Na₂SO₄, and concentrated in vacuo to give the crude product which was then purified by column chromatography (70% EtOAc in Heptane) to afford the title compound (65 mg, 28%) ¹H NMR (400 MHz, d-DMSO) δ 9.07 (s, 1H) 8.15 (s, 1H) 8.04 (dd, 1H) 7.54-7.60 (m, 2H) 7.38-7.48 (m, 2H) 7.30-7.35 (m, 2H) 7.11-7.16 (m, 2H) 7.04-7.09 (m, 2H) 6.90-6.96 (m, 1H) 5.67 (s, 1H) 3.05 (d, 2H) 2.78-2.89 (m, 2H) 2.15 (s, 3H) 2.04 (t, 4H)

Since the compounds of formula (I) are anti-platelet agents, they are useful in a number of therapeutic contexts. For example, the compounds of formula (I) are useful in the treatment or prevention of various thrombotic or thromboembolic diseases or disorders including acute coronary syndromes such as coronary artery disease, myocardial infarction (Ml), unstable angina, thromboembolic stroke, venous thrombosis (including deep vein thrombosis), arterial thrombosis, cerebral thrombosis, pulmonary embolism, cerebral embolism, peripheral occlusive arterial disease (e.g. peripheral arterial disease, intermittent claudication, critical leg ischemia), thromboembolic consequences of surgery, interventional cardiology or immobility, thromboembolic consequences of medication (e.g. hormone replacement therapy), thrombotic consequences of atherosclerotic vascular disease and atherosclerotic plaque formation, transplant atherosclerosis, thromboembolic complications of pregnancy including miscarriage, thromboembolic consequences of thrombophilia, prothrombotic consequences and/or complications of cancer, prevention of thrombosis on artificial surfaces (such as stents, shunts, blood oxygenators, vascular grafts, artificial valves), and restenosis.

The compounds are also effective in treating atheroslerosis and/or in providing a non-surgical therapy that reverses the pathophysiologic basis of atherosclerosis and acute coronary syndrome rather than just providing symptomatic relief. In certain embodiments, the methods provide for the treatment or reduction of coronary atherosclerosis and provide for the promotion of cholesterol efflux from affected vessels. In certain embodiments the methods provide for the promotion of reverse cholesterol transport. In certain embodiments, the affected vessel is a coronary artery. Atheroma volume can be determined by intravascular ultrasound (IVUS). In certain embodiments, the methods provide for a decrease in total plaque volume of an affected vessel. In certain embodiments, the methods provide for a decrease in the average maximal plaque thickness in an affected vessel. In certain embodiments, the methods provide for a decrease in the average maximal atheroma thickness. In certain embodiments, the methods provide for a decrease in plaque volume in least percent plaque area. In certain embodiments, the methods provide for a decrease in the greatest percent plaque area. In certain embodiments, the methods provide for increased mean coronary luminal diameter in an affected vessel. In certain embodiments, the subject administered a compound of the invention can have decreased angiographic lesions as compared with subjects not receiving the compounds of the invention. In certain embodiments, the methods provide a regression in pre-existing lesions. In certain embodiments, the methods and pharmaceutical formulation provide for achieving patency of an occluded vessel or maintaining patency of an occluded vessel.

In one embodiment, the methods provide for the treatment of acute coronary syndromes in patients with signs or symptoms of acute coronary syndromes. In one embodiment, patients can have signs and/or symptoms of myocardial ischemia, for instance, pain in the chest, jaw, arms, or epigastric region, palpitations, shortness of breath, diaphoresis, nausea and/or vomiting. In another embodiment, the methods provide for the treatment of acute coronary syndromes in patients exhibiting signs and symptoms of acute coronary syndromes in conjunction with changes in electrocardiogram (“ECG” or “EKG”), such as ST segment elevations, T wave changes such as inversions, increases in creatine kinase fraction, troponin I or C-reactive protein.

The compounds of the present invention can be used in conjunction with surgical intervention, i.e. before, during, or after surgery. Surgical intervention can include angioplasty, intravascular ultrasound, coronary artery bypass graft (CABG), coronary angiography, implantation of vascular stents, percutaneous coronary intervention (PCI) and/or stabilization of plaques. In certain embodiments, the methods provide for dosing a compound of the present invention before or after surgical intervention to open an occluded vessel, or reduce atherosclerotic plaque in a vessel. Surgical intervention refers to manual, non-pharmacologic or operative methods used for diagnosis, imaging (radiology), prevention, or treatment of the disease or condition. For example, intravascular ultrasound (IVUS) and coronary angiography are procedures that can provide a quantitative assessment of plaque burden (diagnostic purpose), angiography can provide images of vessels (radiologic purpose) and angioplasty can open an occluded vessel (treatment purpose). All are included as surgical interventions as used herein.

The compounds of the present invention are also useful in the treatment of inflammation and/or inflammatory diseases or conditions. Specifically, the compounds of the present invention are useful in treating the inflammatory components of atherosclerotic disease. Inflammatory diseases are characterized by a complex series of histological events, including dilatation of arterioles, capillaries, and venules, with increased permeability and blood flow; exudation of fluids, including plasma proteins; and leukocytic migration into the inflammatory focus. Many forms of inflammation are localized protective responses elicited by injury or destruction of tissues, which serves to destroy, dilute, or wall off both the injurious agent and the injured tissue. The inflammatory response itself is also responsible for pathologic tissue damage. Chronic inflammation is thought to be one of the underlying and sustaining pathologies in cardiovascular disease.

Compounds of the present invention may also be useful as diagnostic agents and adjuncts. For example, the present compounds may be useful in maintaining the reactivity of fractionated whole blood containing platelets such as required for analytical and biological testing or transfusions. In addition, the compounds of the present invention may be useful for maintaining blood vessel patency in conjunction with vascular surgery including bypass grafting, arterial reconstruction, atherectomy, vascular graft and stent patency, organ, tissue, and cell implantation and transplantation. In addition, the compounds of the present invention may be useful for maintaining blood vessel patency in conjunction with interventional cardiology or vascular surgery including bypass grafting, arterial reconstruction, atherectomy, vascular graft and stent patency, organ, tissue, and cell implantation and transplantation.

As used herein, the term “patient” refers to a warm-blooded animal such as a mammal which is afflicted with or at risk of developing a particular thrombotic or thromboembolic disease or disorder. It is understood that guinea pigs, dogs, cats, rats, mice, horses, cattle, sheep, and humans are examples of animals within the scope of the meaning of the term. A patient is in need of treatment for a thrombotic or thromboembolic disease or disorder, atheroslerosis, acute coronary syndromes, inflammation, or an inflammatory disease or condition when the patient is afflicted with one or more of the diseases or disorders described herein or is at a recognized risk of developing one or more of the diseases or disorders described herein as diagnosed by an attending physician or clinician.

The identification of those patients who are in need of treatment of a thrombotic or thromboembolic disease or disorder, atheroslerosis, acute coronary syndromes, inflammation, or an inflammatory disease or condition is well within the ability and knowledge of one skilled in the art. A clinician skilled in the art can readily identify, by the use of clinical tests, physical examination and medical/family history, those patients who are in need of such treatment.

As used herein, the term “therapeutically effective amount” of a compound of formula (I) refers to an amount which is effective in treating a thrombotic or thromboembolic disease or disorder. The term “treating” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment of the thrombotic or thromboembolic disease or disorder, atheroslerosis, acute coronary syndromes, inflammation, or an inflammatory disease or conditions.

A therapeutically effective amount can be readily determined by the attending diagnostician, as one skilled in the art, by the use of conventional techniques and by observing results obtained under analogous circumstances. In determining the therapeutically effective amount, the dose, a number of factors are considered by the attending diagnostician, including, but not limited to: the species of mammal; its size, age, and general health; the specific disease involved; the degree of involvement or the severity of the disease; the response of the individual patient; the particular compound administered; the mode of administration; the bioavailability characteristic of the preparation administered; the dose regimen selected; the use of concomitant medication; and other relevant circumstances.

The compounds of formula (I) can be formulated as pharmaceutical compositions and administered to a mammalian host, such as a human patient in a variety of forms adapted to the chosen route of administration, i.e., orally or parenterally, by intravenous, intramuscular, or subcutaneous routes. Such pharmaceutical compositions can include a compound of formula (I) and a pharmaceutically acceptable carrier and/or adjuvant.

Thus, the present compounds may be systemically administered, e.g., orally, in combination with a pharmaceutically acceptable vehicle such as an inert diluent or an assimilable edible carrier. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavoring agents, preservatives and taste-masking agents.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1″, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release. Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Thus, compounds of the invention may be formulated as a solid, semi-solid, or thixotropic liquid for administration as an implanted depot providing modified release of the active compound. Examples of such formulations include drug-coated stents and poly(glycolide-co-dl-lactide) or PGLA microspheres.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Useful dosages of the compounds of formula (I) can be determined by comparing their in vitro activity, and in vivo activity in animal models. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compounds of the present invention can be administered to a patient at dosage levels in the range of about 0.1 to about 2,000 mg per day. For a normal human adult having a body weight of about 70 kilograms, a dosage in the range of about 0.01 to about 10 mg per kilogram of body weight per day is preferable. However, the specific dosage used can vary. For example, the dosage can depended on a numbers of factors including the requirements of the patient, the severity of the condition being treated, and the pharmacological activity of the compound being used. The determination of optimum dosages for a particular patient is well-known to those skilled in the art.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the compound of formula (I) and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The compounds of the present invention may be used in combination with other therapeutic agents either in discreet dosage forms or in the same pharmaceutical formulation. The compounds of formula (I) may be used in combination (administered either simultaneously, sequentially, or separately) with one or more therapeutic agents including anti-arrythmic agents, anti-hypertensive agents, calcium channel blockers, angiotensin converting enzyme inhibitors, angiotensin-II receptor antagonists, beta-adrenergic receptor blockers, and alpha-adrenergic receptor blockers.

Anti-arrhythmic agents for use in combination with the present compounds include: Class I agents (such as propfenone), Class II agents (such as carbadiol and propranolol), Class III agents (such as sotalol, dofetilide, amiodarone, asimilide, and ibutilide), and Class IV agents (such as diltiazem and verapamil).

Antihypertensive agents include amlodipine and related dihydropyridine compounds, calcium channel blockers, angiotensin coverting enzyme inhibitors (“ACE inhibitors”), angiotensin-II receptor antagonists, beta-adrenergic receptor blockers and alpha adrenergic blockers. Such antihypertensive activity is determined by those skilled in the art according to standard tests (e.g. blood pressure measurements).

Amlodipine and related dihydropyridine compounds are disclosed in U.S. Pat. No. 4,572,909, which is incorporated by reference as if fully set forth. Amlodipine is a potent anti-ischemic and anti-hypertensive agent. U.S. Pat. No. 4,879,303, which is incorporated by reference as if fully set forth, discloses amolodipine benzenesulfonate salt (also termed amlodipine besylate). Amlodipine and amlodipine besylate are potent and long lasting calcium channel blockers. Amlodipine besylate is currently sold in the United States as Norvasc®.

Calcium channel blockers which are within the scope of a combination aspect of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, prenylamine, semotiadil, terodiline, verapamil, aranipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexyline The disclosures of all such U.S. patents are incorporated herein by reference.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, ceronapril, delapril, enalapril, fosinopril, imadapril, lisinopril, moveltopril, perindopril, quinapril, ramipril, spirapril, temocapril, and trandolapril. The disclosures of all such U.S. patents are incorporated herein by reference.

Angiotensin-II receptor antagonists (A-Ii antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, losartan, and valsartan. The disclosures of all such U.S. patents are incorporated herein by reference.

Beta-adrenergic receptor blockers (beta- or. beta.-blockers) which are within the scope of this invention include, but are not limited to: acebutolol, which may be prepared as disclosed in U.S. Pat. No. 3,857,952; alprenolol, amosulalol, which may be prepared as disclosed in U.S. Pat. No. 4,217,305; arotinolol, atenolol, befunolol, betaxolol; The disclosures of all such U.S. patents are incorporated herein by reference.

Alpha-adrenergic receptor blockers (alpha- or .alpha.-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, doxazosin, fenspiride, indoramin, labetolol, naftopidil, nicergoline, prazosin, tamsulosin, tolazoline, trimazosin, and yohimbine, which may be isolated from natural sources according to methods well known to those skilled in the art. The disclosures of all such U.S. patents are incorporated herein by reference.

The compounds of formula (I) may be used in combination with one or more anti-inflammatory or analgesic agents. For example, the P2Y₁ antagonists of the present invention may be administered simultaneously, sequentially or separately in combination with one or more agents selected from:
(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;
(ii) nonsteroidal antiinflammatory drugs (NSAIDs), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;
(iii) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;
(iv) benzodiazepines having a sedative action, e.g. chlordiazepoxide, clorazepate, diazepam, flurazepam, lorazepam, oxazepam, temazepam, triazolam and their pharmaceutically acceptable salts,
(v) H₁ antagonists having a sedative action, e.g. diphenhydramine, pyrilamine, promethazine, chlorpheniramine, chlorcyclizine and their pharmaceutically acceptable salts;
(vi) miscellaneous sedatives such as glutethimide, meprobamate, methaqualone, dichloralphenazone and their pharmaceutically acceptable salts;
(vii) skeletal muscle relaxants, e.g. baclofen, carisoprodol, chlorzoxazone, cyclobenzaprine, methocarbamol, orphrenadine and their pharmaceutically acceptable salts,
(viii) NMDA receptor antagonists, e.g. dextromethorphan ((+)-3-hydroxy-N-methylmorphinan) and its metabolite dextrorphan ((+)-3-hydroxy-N-methylmorphinan), ketamine, memantine, pyrroloquinoline quinone and cis-4-(phosphonomethyl)-2-piperidinecarboxylic acid and their pharmaceutically acceptable salts;
(ix) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl) quinazoline;
(x) tricyclic antidepressants, e.g. desipramine, imipramine, amytriptiline and nortriptiline;
(xi) anticonvulsants, e.g. carbamazepine and valproate;
(xii) Tachykinin (NK) antagonists, particularly Nk-3, NK-2 and NK-1 e.g. antagonists, (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholiny]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)
(xiii) Muscarinic antagonists, e.g oxybutin, tolterodine, propiverine, tropsium chloride and darifenacin;
(xiv) COX-2 inhibitors, e.g. celecoxib, rofecoxib and valdecoxib;
(xv) Non-selective COX inhibitors (preferably with GI protection), e.g. nitroflurbiprofen (HCT-1026);
(xvi) coal-tar analgesics, in particular, paracetamol;
(xvii) neuroleptics, such as droperidol;
(xviii) Vanilloid receptor agonists, e.g. resinferatoxin;
(xix) Beta-adrenergic compounds such as propranolol;
(xx) Local anaesthetics, such as mexiletine;
(xxi) Corticosteriods, such as dexamethasone
(xxii) serotonin receptor agonists and antagonists;
(xxiii) cholinergic (nicotinic) analgesics;
(xxiv) miscellaneous agents such as Tramadol®;
(xxv) PDEV inhibitors, such as sildenafil, vardenafil or taladafil;
(xxvi) serotonin reuptake inhibitors, e.g. fluoxetine, paroxetine, citalopram and sertraline;
(xxvii) mixed serotonin-noradrenaline reuptake inhibitors, e.g. milnacipran, venlafaxine and duloxetine;
(xxviii) noradrenaline reuptake inhibitors, e.g. reboxetine;
(xxix) alpha-2-delta ligands, e.g. gabapentin and pregabalin.

The ability of a compound of the present invention to act as an antagonist of P2Y₁ is demonstrated using pharmacological models that are well known in the art, for example, using models such as the tests described below.

### Conducting P2Y, Receptor Binding Assay

### Materials and Methods

[β−³³P]-2-thiomethyl ADP (2100 Ci/mmol) was purchased from PerkinElmer, Protease Inhibitors Complete, EDTA-free, was purchased from Roche, and ADP (Adenosine diphosphate), MRS2179, and 2-thiomethy ADP (2-Me-S-ADP) were purchased from Sigma Chemical Co.

Binding of 0.3 nM [β-³³P]-2-thiomethyl ADP to platelet P2Y₁ receptor in 132N1 cell membrane was carried out in triplicate at 25° C. with 96 well plate (Corning, 3365). Nonspecific binding was defined as binding in the presence of 1 μM of 2-Me-S-ADP. One 1 L of dimethyl sulfoxide (DMSO) or 1 μL of DMSO (Sigma-Aldrich, 472301) containing a test compound diluted to final assay concentrations of 0.1 nM to 1 mM were placed into each well of a Corning 96-well plate. A volume of 60 μL of buffer: 50 mM Tris pH 7.5 (Sigma, T-2194), 1 mM EDTA (Gibco, 15575-038) and 100 mM NaCl (Gibco, 200740-011) containing 0.3 nM [β-³³P]-2-thiomethyl ADP 40 μL of hP2Y₁ expressing 132N1 cell membrane prep was added to each well. After incubation on shaker for 60 minutes, The contents of the wells are filtered and washed 8 times with Tomtec Cell Harvester through UniFilter 96, GF/B plates (PerkinElmer, 6005177) Forty five microliters (45 mL) Scintillation cocktail (Ultima-Flo-M) was added into each well and plates counted on a Trilux Microbeta Counter.

### Procedure

1. Add 1 μL DMSO or compounds into the wells according to the protocol.
2. Add 60 μL incubation buffer with [β-³³P]-2-thiomethyl ADP into each well.
3. Add 40 μL hP2Y₁ expressing 132N1 cell membrance prep. Incubate 60 minutes on top of the shaker (IKA Shutter MTS4).
4. Filter and wash 8× with Tomtec Cell Harvester through UniFilter GF/B plates (PerkinElmer, 6005177).
5. Add 45 μL scintillation cocktail into each well and plates counted on a Trilux Microbeta Counter.

### Functional Platelet Aggregation Assay

### Step 1. Preparing Gel Filtered Human Platelets

### Column Preparation:

1. Wash the Sepharose 2B (Amersham, cat. # 17-0130-01) in acetone (3-4 volumes) followed by 0.9% NaCl (5-6 volumes).
2. Place rubber ring (Small parts Inc. ORS-020-05) at bottom of a 60 ml monoject syringe.
3 Place 41 μm filter (Millipore, cat. # 4102500) and stainless steel filter support (Millipore XX30025-10) on top of rubber ring.
4. Pour Sepharose 2B into column being careful to avoid air bubbles in the poured column.
5. Add Sepharose 2B until column is full (50-55 ml).
6. Allow the matrix to just barely dry before beginning to wash with Buffer I.
7. Wash column with approximately 150 ml Buffer I.
8. Before gel-filtering platelets, wash column with 4 volumes of Buffer I plus BSA/Glucose to equilibrate column.
9. The column should be used at room temperature.

### Platelet Preparation for Gel-Filtration:

Blood collection: For optimal assay results, use freshly collected human blood from healthy, drug-free adult volunteers drawn into 5-ml vacutainer tubes (preloaded with sodium citrate as the anticoagulant). Blood samples should be processed as soon as possible for best platelet function.
1. Collect blood from a forearm vein using a 19-gauge butterfly catheter. Discard the first 2 ml of blood before collecting into 5-ml vacutainer tubes.
2. Blood should be mixed by inversion immediately.

### Isolation of Platelets:

1. Spin at 1000 rpm (200×g) for 10 min in a Sorvall RT 7 centrifuge with the RTH-250 rotor. It is critical that the spin is performed at room temperature so as not to activate the platelets. Draw off the supernatant to obtain PRP.
2. Apply PRP (5-6 ml) to column and allow PRP to run into Sepharose 2B gel.
3. Allow the matrix to just barely dry before adding more Buffer I plus BSA/Glucose.
4. Collect platelets (1 ml/tube) and count. Add together platelet suspensions of sufficient concentration for experiment.
5. Adjust platelet count to 3×10⁸ platelets/ml using Buffer I plus BSA/Glucose.
6. Allow platelets to rest at 37° C. for at least 15 minutes prior to use.
7. Gel-Filtered platelets are now ready for use in assay.

### Step 2. Procedure for Platelet Aggregation Assay.

1. Add 250 μl of platelet suspension (3×10⁸ platelets/ml) to the wells of a 96-well flat bottom microtiter plate (Corning, Costar #3596). Add 250 μl buffer to 2 blank wells.
2. Add 2 μl compound solution (diluted in 100% DMSO).
3. Mix using Titer Plate Shaker (Lab-line Instruments, Inc.) at room temperature for 10 min at Speed 7.
4. Add fibrinogen (0.22 mg/ml final concentration) to all wells (exclude blank wells).
5. Mix using Titer Plate Shaker at room temperature for 2 min at Speed 7.
6. Add 5 μl ADP (150 μM working solution) to all wells (exclude blank wells).
7. Read immediately at A490 nm, 5 min.

### In Vivo Rat Model

In the anesthetized rats, a tracheotomy is preformed and a 16 to 18 gauge, 2 to 3 cm metal tracheal tube is inserted just slightly beyond the thoracic inlet to maintain airway patency. The shortest length but largest size diameter tube that can be quickly inserted into the trachea is used. Rat body temperature is maintained at 37° C. The incision for the tracheotomy is then enlarged to expose the lift common carotid artery region. The carotid is then isolated and the vagus nerve and fascia is dissected away from the vessel. Baseline carotid artery flow is determined using a Transonic Systems Model T106 flow meter and a flow probe that is placed around the vessel. A thrombus is then induced by applying two pieces of filter paper (Grade 1, Whatman) either 1.5 or 2 mm in diameter depending on the diameter of the vessel, saturated with 30% ferric chloride solution immediately proximal to the flow probe. The pieces of filter paper are placed on opposite sides of the carotid artery (one beneath and one on top) in contact with the adventitial surface. Two pieces of filter paper are used to create a very uniform injury around the entire vessel. After 10 minutes the filter paper is removed and the area is flooded with warmed saline to once again monitor blood flow. Data is only used from the rat that after the application of the ferric chloride, has a carotid blood flow that is equal to, or higher then, the baseline blood flow (acceptable baseline blood flows were >4 mL/minute). Time to thrombotic occlusion as defined as blood flow declining to 0 ml/minute, is monitored for 60 minutes from the initiation of the injury. For intravenous drug administration the compound is administered after the rat is anesthetized. A normal vessel occludes in approximately 15 minutes after the ferric chloride is applied. At the end of the study a blood sample is taken to determine plasma drug levels and for platelet aggregation assay. The in vivo efficacy endpoints are the time to occlusion and thrombus weight. The bleeding time, ex-vivo platelet aggregation, and plasma concentration of test compound are also measured.

All publications, including but not limited to, issued patents, patent applications, and journal articles, cited in this application are each herein incorporated by reference in their entirety. Although the invention has been described above with reference to the disclosed embodiments, those skilled in the art will readily appreciate that the specific experiments detailed are only illustrative of the invention. It should be understood that various modifications can be made without departing from the spirit of the invention.

P2Y1 BINDING DATA FOR EXAMPLES 1-129

| | P2Y1 IC₅₀ |
|---|---|
| EXAMPLE | (UM) |
| 1 | 0.152 |
| 2 | 0.089 |
| 3 | 0.048 |
| 4 | 0.089 |
| 5 | 0.459 |
| 6 | 0.095 |
| 7 | 0.122 |
| 8 | 0.128 |
| 9 | 0.253 |
| 10 | 0.188 |
| 11 | 0.991 |
| 12 | 0.207 |
| 13 | 0.246 |
| 14 | 0.133 |
| 15 | 0.283 |
| 16 | 0.285 |
| 17 | 0.307 |
| 18 | 0.731 |
| 19 | 0.142 |
| 20 | 0.150 |
| 21 | 0.272 |
| 22 | 0.139 |
| 23 | 8.99 |
| 24 | 0.306 |
| 25 | 0.268 |
| 26 | >10.0 |
| 27 | 7.22 |
| 28 | >10.0 |
| 29 | >10.0 |
| 30 | 1.24 |
| 31 | 3.33 |
| 32 | >10.0 |
| 33 | >10.0 |
| 34 | 0.800 |
| 35 | 1.19 |
| 36 | 1.30 |
| 37 | 1.32 |
| 38 | 1.91 |
| 39 | 2.22 |
| 40 | 2.74 |
| 41 | 5.06 |
| 42 | 6.98 |
| 43 | 7.56 |
| 44 | 7.79 |
| 45 | 8.85 |
| 46 | >10.0 |
| 47 | >10.0 |
| 48 | >10.0 |
| 49 | >10.0 |
| 50 | >10.0 |
| 51 | >10.0 |
| 52 | >10.0 |
| 53 | >10.0 |
| 54 | >10.0 |
| 55 | >10.0 |
| 56 | >10.0 |
| 57 | >10.0 |
| 58 | >10.0 |
| 59 | >10.0 |
| 60 | >10.0 |
| 61 | >10.0 |
| 62 | >10.0 |
| 63 | >10.0 |
| 64 | >10.0 |
| 65 | >10.0 |
| 66 | >10.0 |
| 67 | >10.0 |
| 68 | >10.0 |
| 69 | >10.0 |
| 70 | >10.0 |
| 71 | >10.0 |
| 72 | >10.0 |
| 73 | >10.0 |
| 74 | >10.0 |
| 75 | >10.0 |
| 76 | >10.0 |
| 77 | >10.0 |
| 78 | >10.0 |
| 79 | >10.0 |
| 80 | >10.0 |
| 81 | >10.0 |
| 82 | >10.0 |
| 83 | 3.39 |
| 84 | >10.0 |
| 85 | >10.0 |
| 86 | 5.06 |
| 87 | >10.0 |
| 88 | 0.149 |
| 89 | 0.054 |
| 90 | 0.039 |
| 91 | 0.087 |
| 92 | 0.182 |
| 93 | 0.123 |
| 94 | 8.78 |
| 95 | 0.689 |
| 96 | 0.176 |
| 97 | 0.339 |
| 98 | 0.068 |
| 99 | >10.0 |
| 100 | >10.0 |
| 101 | 1.02 |
| 102 | 1.19 |
| 103 | 0.564 |
| 104 | 0.022 |
| 105 | >10.0 |
| 106 | 0.287 |
| 107 | 0.060 |
| 108 | 0.012 |
| 109 | 0.198 |
| 110 | 0.290 |
| 111 | 0.133 |
| 112 | >10.0 |
| 113 | 0.057 |
| 114 | 0.012 |
| 115 | 0.018 |
| 116 | 1.12 |
| 117 | >10.0 |
| 118 | >3.33 |
| 119 | 0.088 |
| 120 | >10.0 |
| 121 | >10.0 |
| 122 | >10.0 |
| 123 | 0.121 |
| 124 | 0.032 |
| 125 | 0.182 |
| 126 | 0.170 |
| 127 | 0.158 |
| 128 | 0.763 |
| 129 | >10.0 |

1. A compound of the formula 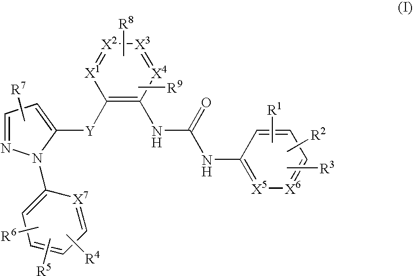
wherein
X¹, X², X³, X⁴, X⁵, X⁶, and X⁷ are each independently CH or N, with the proviso that no more than two of X¹, X², X³, X⁴ can be N at the same time;
Y is oxy or thio;
R¹, R², R³, R⁴, R⁵, and R⁶ are each independently —H, C₁-C₆ alkyl, C₅-C₈ cycloalkyl, cycloheteroalkyl, hydroxy, C₁-C₆ alkoxy, halo, —CF₃, —CF₂CF₃, —OCF₃, —OCF₂CF₃, —OCF₂CF₂H, optionally substituted phenyl, —SiMe₃, —(CR¹⁰R¹¹), —OR¹², —SR¹³, —CN, —NO₂, —(CR¹⁰R¹¹)ₙNR¹⁴R¹⁵ (CR¹⁰R¹¹)—C(O)R¹², —(CR¹⁰R¹¹)ₙ—CO₂R¹², —(CR¹⁰R¹¹)ₙ—C(O)—NR¹⁴R¹⁵, or —S(O)ₚR¹⁶;
R⁷ is —H, C₁-C₄ alkyl, halo, —CF₃, or —(CR¹⁰R¹¹)—CO₂R¹²;
R⁸ and R⁹ are each independently —H, C₁-C₆ alkyl, hydroxy, C₁-C₆ alkoxy, halo, —CF₃, or —SR¹³ and are each only bound to a carbon atom;
R¹⁰ and R¹¹ are each independently at each occurrence —H, C₁-C₄ alkyl, or halo;
R¹² and R¹³ are each independently at each occurrence —H or C₁-C₆ alkyl;
R¹⁴ and R¹⁵ are each independently at each occurrence —H, C₁-C₆ alkyl, —C(O)(C₁-C₆ alkyl), —S(O)ₚ(C₁-C₆ alkyl), or R¹⁴ and R¹⁵, taken together in combination with the nitrogen to which they are attached combine to form a piperidinyl or pyrrolidinyl ring;
R¹⁶ is —H, C₁-C₄ alkyl;
n, at each occurrence, is selected from 0, 1, 2, 3, and 4; and
p, at each occurrence, is selected from 0, 1, and 2;
or a stereoisomer or a pharmaceutically acceptable salt thereof.

2. A compound according to claim 1 wherein R¹ is C₁-C₆ alkyl, —F, —Cl, —Br, —I, —OCF₃, —OCF₂CF₃, —OCF₂CF₂H, —SR¹³, or —(CR¹⁰R¹¹)ₙ—CO₂R¹²; or a stereoisomer or a pharmaceutically acceptable salt thereof.

3. A compound according to either of claims 1 or 2 wherein R² and R³ are each independently —H, —F, —Cl, methyl, or methoxy; or a stereoisomer or a pharmaceutically acceptable salt thereof.

4. A compound according to either of claims 1 or 2 wherein R² and R³ are each —H; or a stereoisomer or a pharmaceutically acceptable salt thereof.

5. A compound according to claim 3 wherein R⁴ is C₁-C₆ alkyl, C₁-C₆ alkoxyl —F, —Cl, —Br, —I, —CF₃, —CF₂CF₃, or —(CR¹⁰R¹¹)ₙ—CO₂R¹²; or a stereoisomer or a pharmaceutically acceptable salt thereof.

6. A compound according to claim 5 wherein R⁵ and R⁶ are each —H; R⁷ is —H or C₁-C₄ alkyl; and R⁸ and R⁹ are both —H; or a stereoisomer or a pharmaceutically acceptable salt thereof.

7. A compound according to claim 6 wherein X¹ and X³ are each independently CH or N and X², X⁴, X⁵, X⁶, and X⁷ are all CH; or a stereoisomer or a pharmaceutically acceptable salt thereof.

8. A compound according to claim 7 wherein R¹ is —OCF₃ or t-butyl; R², R³, R⁵, R⁶, R⁸, and R⁹ are all —H; R⁴ is methyl, ethyl, methoxy, ethoxy, —F, —Cl, or —CF₃; and R⁷ is —H or C₁-C₄ alkyl; or a stereoisomer or a pharmaceutically acceptable salt thereof.

9. A compound of formula (IA) 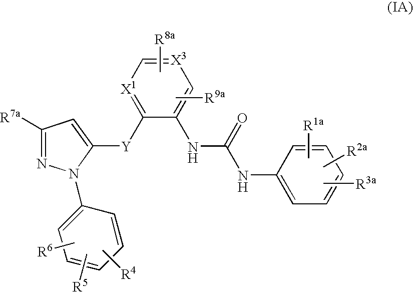
wherein X1 and X3 are each independently CH or N; Y is oxy or thio; R1a, R2a, and R3a are each independently —H, C1-C6 alkyl, C1-C6 alkoxy, halo, —CF3, —OCF3, —SR13, and —(CR10R11)ₙ—CO2R12; R4, R5, and R6 are each independently —H, C1-C6 alkyl, C5-C8 cycloalkyl, cycloheteroalkyl, hydroxy, C1-C6 alkoxy, halo, —CF3, —CF2CF3, —OCF3, —OCF2CF3, —OCF2CF2H, optionally substituted phenyl, —SiMe3, —(CR10R11)n-OR12, —SR13, —CN, —NO2, —(CR10R11)nNR14R15, —(CR10R11)n-C(O)R12, —(CR10R11)nCO2R12, —(CR10R11)n-C(O)—NR14R15, or —S(O)pR16; R7a is —H, C1-C4 alkyl, halo, or —CF3; R8a and R9a are each independently —H, C1-C6 alkyl, hydroxy, C1-C6 alkoxy, halo, or —CF3; R10 and R11 are each independently at each occurrence —H, C1-C4 alkyl, or halo; R12 and R13 are each independently at each occurrence —H or C1-C6 alkyl; R14 and R15 are each independently at each occurrence —H, C1-C6 alkyl, —C(O)(C1-C6 alkyl), —S(O)p(C1-C6 alkyl), or R¹⁴ and R¹⁵, taken together in combination with the nitrogen to which they are attached combine to form a piperidinyl or pyrrolidinyl ring; R¹⁶ is —H, C1-C4 alkyl; n, at each occurrence, is selected from 0, 1, 2, 3, and 4; and p, at each occurrence, is selected from 0, 1, and 2; or a stereoisomer or pharmaceutically acceptable salt thereof.

10. A compound according to claim 9, or a stereoisomer or a pharmaceutically acceptable salt thereof, wherein R^{1a} is —C₁-C₆ alkyl, —F, —Cl, —OCF₃, —SR¹³, or —(CR¹⁰R¹¹)ₙ—CO₂R¹²; both R^{2a} and R^{3a} are —H; R^{4a} is —H, C₁-C₆ alkyl, C₁-C₆ alkoxyl —F, —Cl, —Br, —I, —CF₃, —CF₂CF₃, or —(CR¹⁰R¹¹)ₙ—CO₂R¹²; and R^{5a}, R^{6a}, R^{8a}, and R^{9a} are all —H; or a stereoisomer or a pharmaceutically acceptable salt thereof.

11. A compound of the formula 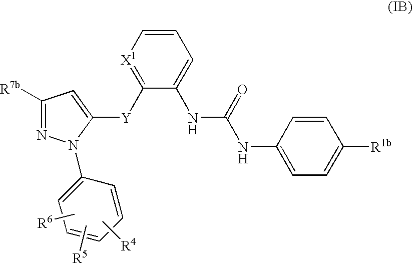
wherein X¹ is CH or N; Y is oxy or thio; R^{1b} is —H, C₁-C₆ alkyl, C₁-C₆ alkoxy, halo, —CF₃, —OCF₃, —SR¹³, and —(CR¹⁰R¹¹)ₙ—CO₂R¹²; R⁴, R⁵, and R⁶ are each independently —H, C₁-C₆ alkyl, C₅-C₈ cycloalkyl, cycloheteroalkyl, hydroxy, C₁-C₆ alkoxy, halo, —CF₃, —CF₂CF₃, —OCF₃, —OCF₂CF₃, —OCF₂CF₂H, optionally substituted phenyl, —SiMe₃, —(CR¹⁰R¹¹)ₙ—OR¹², —SR¹³, —CN, —NO₂, —(CR¹⁰R¹¹)ₙNR¹⁴R¹⁵, —(CR¹⁰R¹¹)ₙ—C(O)R¹², —(CR¹⁰R¹¹)ₙ—CO₂R¹², —(CR¹⁰R¹¹)ₙ—C(O)—NR¹⁴R¹⁵, or —S(O)ₚR¹⁶; R^{7b} is —H, C₁-C₄ alkyl, halo, or —CF₃; R¹⁰ and R¹¹ are each independently at each occurrence —H, C₁-C₄ alkyl, or halo; R¹² and R¹³ are each independently at each occurrence —H or C₁-C₆ alkyl; R¹⁴ and R¹⁵ are each independently at each occurrence —H, C₁-C₆ alkyl, —C(O)(C₁-C₆ alkyl), —S(O)ₚ(C₁-C₆ alkyl), or R¹⁴ and R¹⁵, taken together in combination with the nitrogen to which they are attached combine to form a piperidinyl or pyrrolidinyl ring; R¹⁶ is —H, C₁-C₄ alkyl; n, at each occurrence, is selected from 0, 1, 2, 3, and 4; and p, at each occurrence, is selected from 0, 1, and 2; or a stereoisomer or a pharmaceutically acceptable salt thereof.

12. A compound according to claim 11 wherein Y is oxy; R^{1b} is optionally substituted phenyl, C₁-C₆ alkyl or —OCF₃; R⁴ is C₁-C₆ alkyl, C₁-C₆ alkoxy, —F, —Cl, —Br, —I, —CF₃, —CF₂CF₃, or —(CR¹⁰R¹¹)ₙ—CO₂R¹²; R⁵ and R⁶ are each —H; and R^{7b} is C₁-C₄ alkyl or halo; or a stereoisomer or a pharmaceutically acceptable salt thereof.

13. A compound according to either of claims 11 or 12 wherein X¹ is CH; or a stereoisomer or a pharmaceutically acceptable salt thereof.

14. A compound according to claim 13 wherein R¹⁰, R¹¹, R¹², and R¹³ are all —H in all occurrences; or a stereoisomer or a pharmaceutically acceptable salt thereof.

15. A compound according to claim 1 selected from the group of compounds represented by the formulae 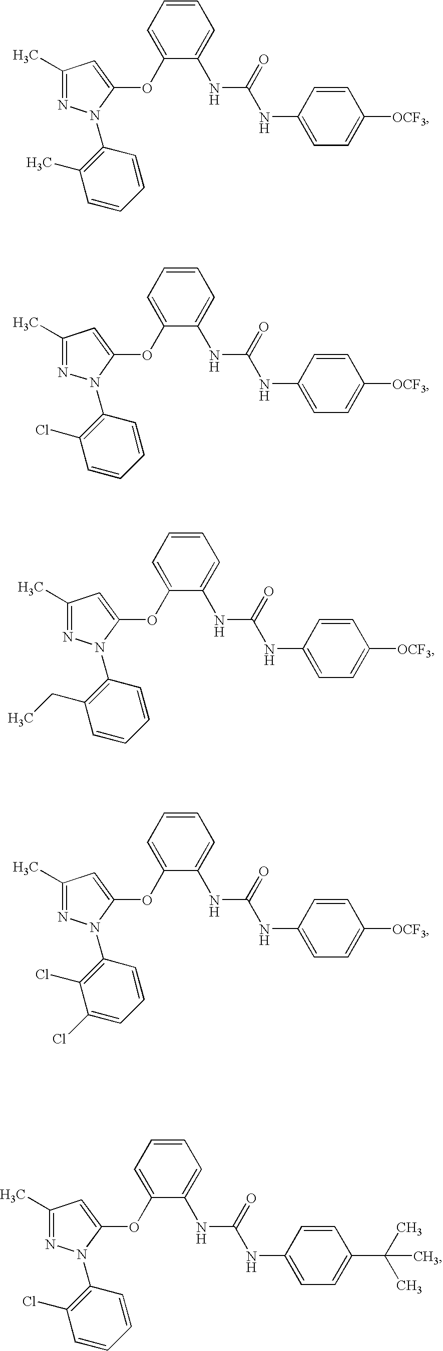 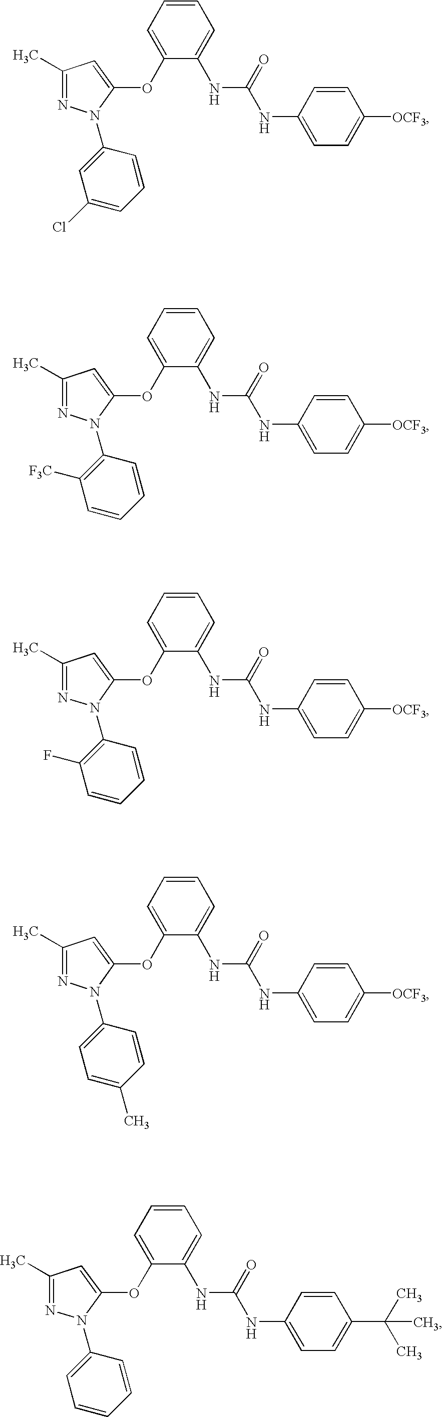 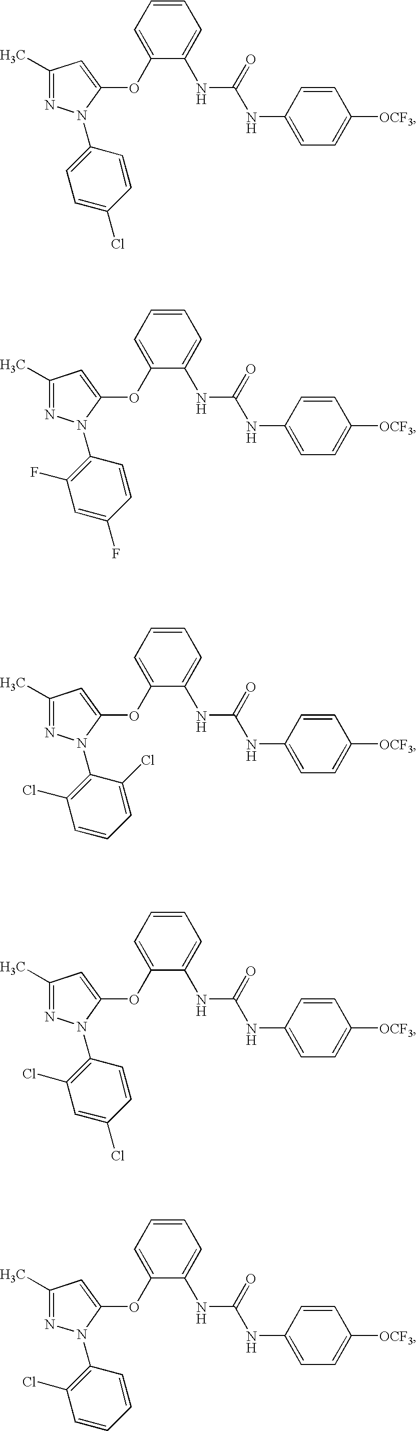 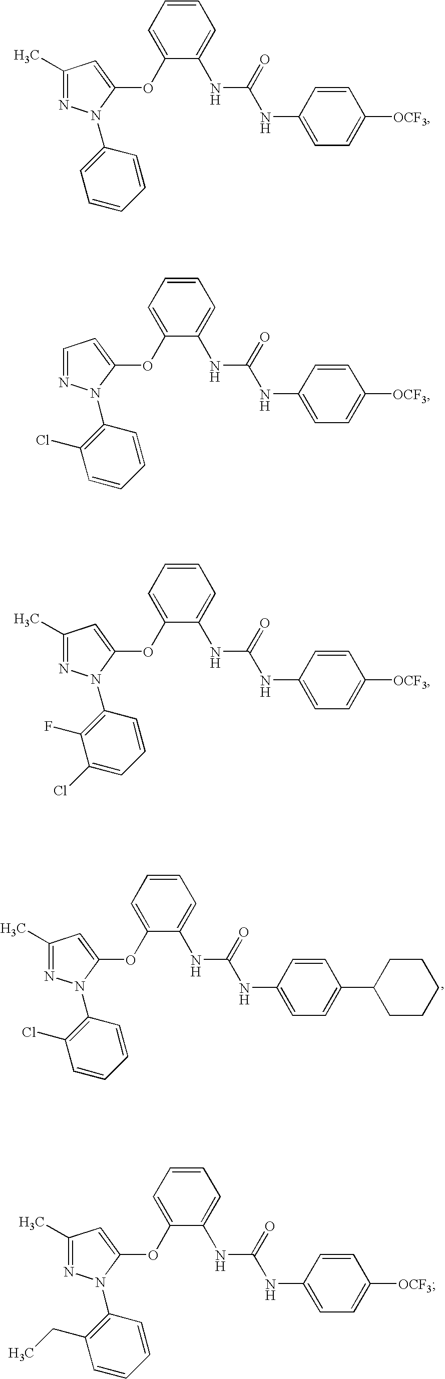 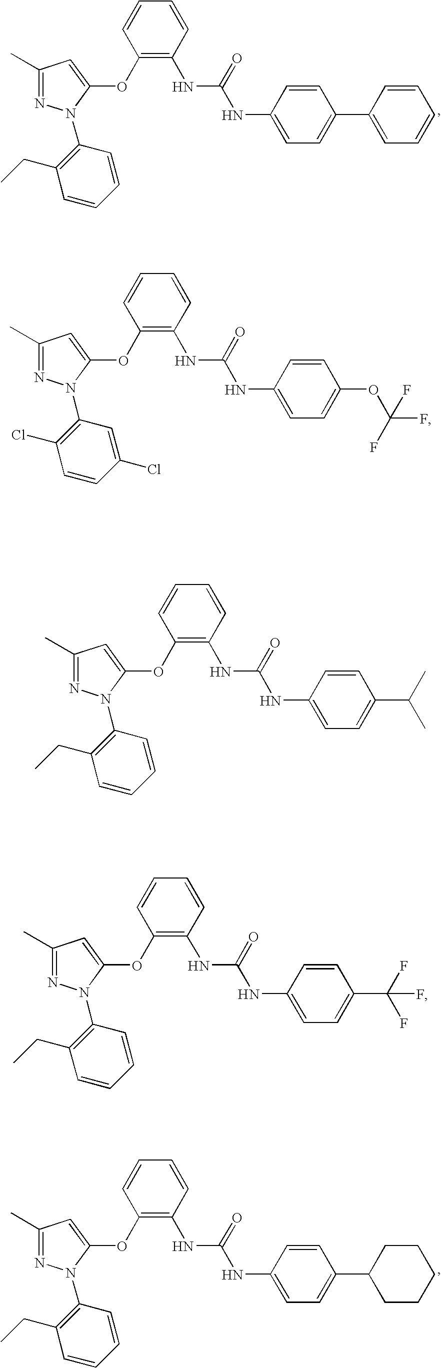 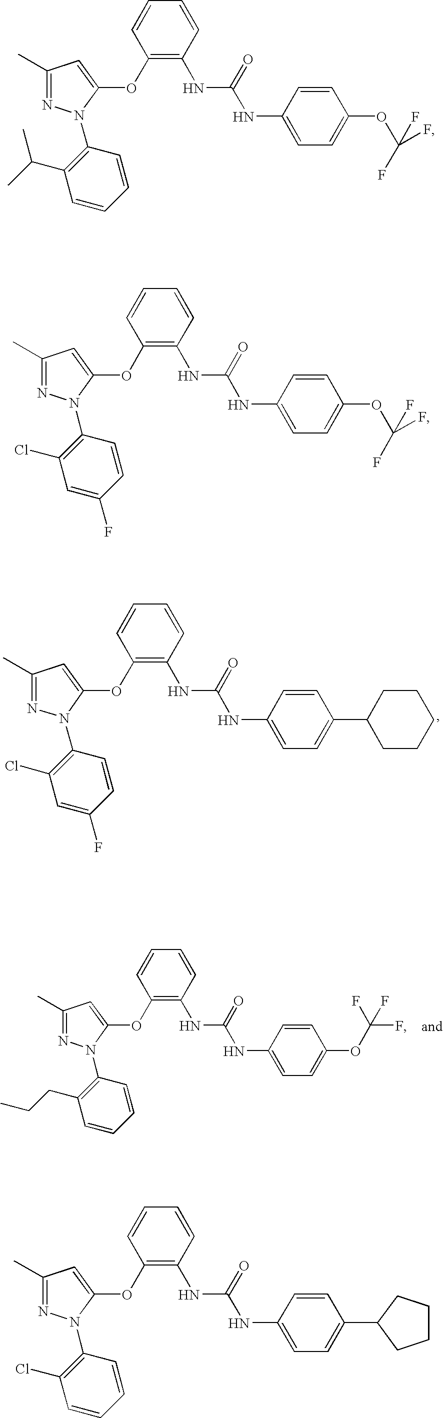
or a pharmaceutically acceptable salt thereof.

16. A compound according to claim 1 selected from the group of compounds represented by the formulae 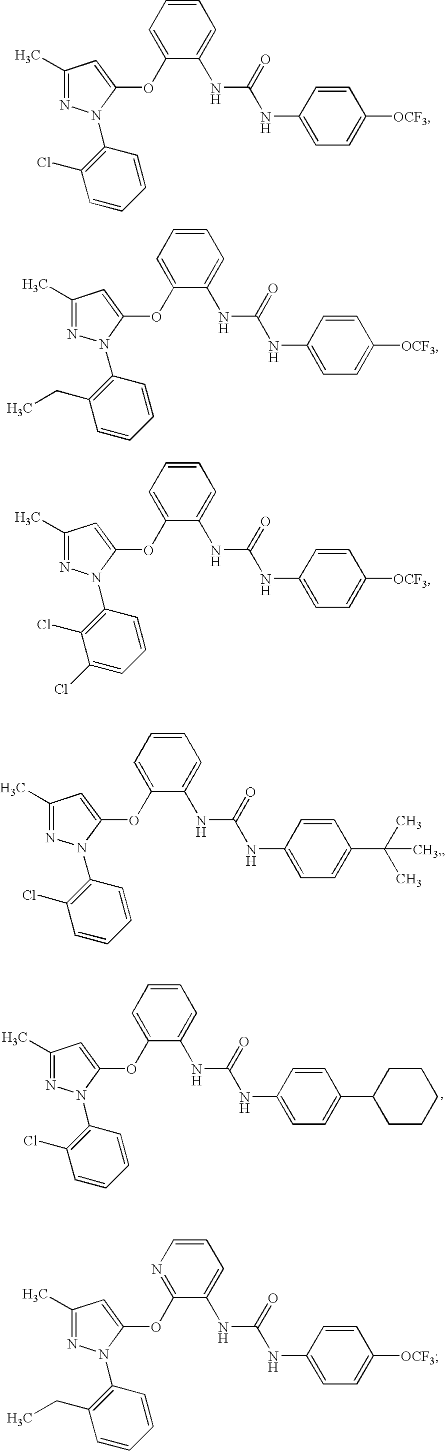
or a pharmaceutically acceptable salt thereof.

17. A compound according to claim 1 which is 1-{2-[2-(2Chloro-phenyl)-5-methyl-2H-pyrazol-3-yloxy]-phenyl}3-(4-trifluoromethoxy-phenyl)-urea, or a pharmaceutically acceptable salt thereof.

18. A compound represented by the formula 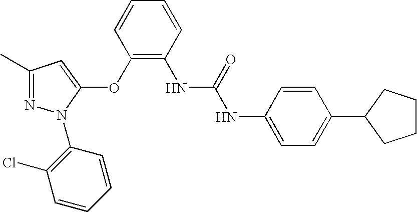
or a pharmaceutically acceptable salt thereof.

19. A pharmaceutical composition comprising a compound according to any of claims 15 to 18, or a stereoisomer or a pharmaceutically acceptable salt thereof, and a pharmaceutically acceptable carrier.

20. A method for modulating platelet activity in a patient in need thereof comprising administering to said patient a therapeutically effective amount of a compound according to any of claims 15 to 18, or a stereoisomer or a pharmaceutically acceptable salt thereof.

21. A method of treating a thromboembolic disorder in a patient in need thereof comprising administering to said patient a therapeutically effective amount of a compound according to any of claims 15 to 18, or a stereoisomer or a pharmaceutically acceptable salt thereof.

22. A method of treating atherosclerosis in a patient in need thereof comprising administering to said patient a therapeutically effective amount of a compound according to any of claims 15 to 18, or a stereoisomer or a pharmaceutically acceptable salt thereof.

23. A method of treating an acute coronary syndrome in a patient with signs or symptoms of an acute coronary syndrome comprising administering to said patient a therapeutically effective amount of a compound according to any of claims 15 to 18, or a stereoisomer or a pharmaceutically acceptable salt thereof.
